(19) United States
(12) Patent Application Publication
(10) Pub. No.: US 2023/0127258 A1
(43) Pub. Date: Apr. 27, 2023

(54) COMPOSITION COMPRISING THROMBIN DERIVED PEPTIDES AND USE THEREOF

(71) Applicant: In2Cure AB, Lund (SE)

(72) Inventors: Artur Schmidtchen, Lund (SE); Manoj Puthia, Lund (SE); Ganna Petruk, Lund (SE)

(21) Appl. No.: 17/783,958

(22) PCT Filed: Nov. 18, 2020
(86) PCT No.: PCT/EP2020/082581

§ 371 Date: Jun. 9, 2022

(30) Foreign Application Priority Data
Dec. 18, 2019 (EP) 19217464.7

(51) Int. Cl. A61K 38/48; A61K 47/10; A61P 31/00; A61K 47/18; A61K 47/69

(52) U.S. Cl. CPC A61K 38/4833; A61K 47/10; A61P 31/00; A61K 47/183; A61K 47/6903

(57) ABSTRACT

The present invention relates to compositions comprising: a) a thrombin derived peptide and, b) a non-ionic polymer capable of forming a hydrogel and c) an aqueous solution. The invention also provides compositions comprising: a) a thrombin derived peptide and, b) EDTA and c) an aqueous buffer. The invention also provides compositions comprising: a) a thrombin derived peptide in high concentration. A product comprising the compositions, as well as the compositions or the products for use in a method of treatment are also disclosed.

### FIELD OF INVENTION

The present invention relates to the fields of local treatment of disorders, in particular to topical treatment of disorders associated with or at risk of becoming associated with infection and/or inflammation. The invention also relates to the field of compositions useful for such treatments. In one embodiment, the present invention relates to a non-ionic hydrogel polymer based composition containing thrombin derived peptides with antibacterial and anti-inflammatory function.

### BACKGROUND OF THE INVENTION

Wounds of various types have an immense and significant impact on patients, health care, and society. Types of wounds include acute post-surgical wounds and burns, and large patient groups have non-healing ulcers resulting from diabetes or circulatory disturbances. With a point prevalence of around 2 per 1000, costs for chronic wounds are substantial and account for 1-3% of the total health system costs in developed countries. Considering burns, 67 million injuries were reported in 2015, resulting in about 2.9 million hospitalizations and 176.000 deaths. In a study published 2014, the mean total costs for burn care in high income countries was estimated to around 88000 USD per patient.

From a physiological perspective, wound healing is an evolutionarily conserved physiological sequence of biologically interlinked events. An initial phase of hemostasis is followed by phases of inflammation, proliferation, and tissue remodeling. Initial surveillance mediated by human innate immunity is instrumental in the control of bacteria during wounding, and lipopolysaccharide sensing by Toll-like receptors (TLR) is crucial in early responses to infection. However, an excessive TLR response causes localized and sometimes excessive inflammation, as observed in postoperative infections, infected burn wounds, or non-healing ulcers. All these wound complications delay proper healing, increasing the risk of severe infections and potentially leading to scar formation. Prophylactic use of systemic antibiotics can reduce the incidence of wound and surgical infections. However, this use of antibiotics drives the development of resistance, and infections caused by antibiotic-resistant strains of Staphylococcus aureus and Pseudomonas aeruginosa, which are bacteria that cause postoperative infections and infections in chronic wounds and burns, presents a major challenge. For example, in European hospitals, the overall rates of surgical site infection (SSI) range between 3% and 4% of patients undergoing surgery. Depending on the nature of surgery in question, the incidence of SSI ranges between <1% to >10%. In the future, as the population ages, the incidence of SSI is expected to sharply increase because the incidence is associated with age, with a doubling of the rate in patients older than 64 years. Besides antibiotic treatment, today's strategies to counteract wound infection involve functionalization of gels, dressings, or biomaterials with various anti-infective components. Commonly used additives in the clinic include silver and polyhexanide (polyhexamethylene biguanide, PHMB), which is used on acute wounds and burns, and on non-healing ulcers. Although such treatments can kill the bacteria, they do not address the associated inflammatory component. Conversely, treatments addressing inflammation mainly aim to inactivate and scavenge proteases, such as gelatin-based wound dressings. Thus, today's wound care only addresses one issue (the infection or protease action), and there are no therapeutic modalities currently available that both control bacteria and target the origins and causes of excessive infection-inflammation in wounds or surgical settings.

Because wound healing is important for survival, it is not surprising that multiple natural host defense systems are activated during injury involving initial hemostasis and clot formation and that proteins and peptides are activated in our innate immune system. In humans, examples of such host defense systems include neutrophil-derived α-defensins and the cathelicidin LL-37 and proteolytic products of plasma proteins such as thrombin. Thrombin, which is initially formed by selective proteolysis by coagulation factor X, mediates fibrinogen degradation and clot formation in the acute wounding phase. However, subsequent proteolysis leads to formation of fragments of about 11 kDa, which mediate aggregation of lipopolysaccharide (LPS) and bacteria, facilitating endotoxin clearance and microbial killing. Further proteolysis leads to formation of smaller thrombin-derived C-terminal peptides (TCP) of roughly 2 kDa, such as FYT21 (FYTHVFRLKKWIQKVIDQFGE) (SEQ ID NO 2) and HVF18 (HVFRLKKWIQKVIDQFGE) (SEQ ID NO 4), which are present in human wound fluids, and have been demonstrated to exert anti-endotoxic functions in vitro and in vivo. The peptide TCP-25 (GKYGFYTHVFRLKKWIQKVIDQFGE), SEQ ID NO: 1, which encompasses these endogenous sequences, is antimicrobial and binds to and neutralizes bacterial LPS and protects against P. aeruginosa-induced sepsis and LPS-mediated shock in experimental animal models, mainly via reduction of systemic cytokine responses. Moreover, the peptide interacts directly with monocytes and macrophages and inhibits TLR4- and TLR2-induced NF-kB activation in response to several microbe-derived agonists. Additionally, the peptide reduces inflammatory responses to intact bacteria during phagocytosis and inhibits neutrophil responses to LPS in vitro and in vivo.

EP1987056 discloses the TCP-25 peptide and various variations thereof.

EP2480567 discloses use of the TCP-25 peptide and various variations thereof.

### SUMMARY OF THE INVENTION

There is however a need for suitable, stable and effective formulations and compositions for delivery of the TCP-25 peptide and similar peptides. In particular, there is a need for pharmaceutical formulations, which are useful for local treatment, e.g. for topical treatment. There is also a need for pharmaceutical formulations comprising TCP-25 peptides with high stability. There is also a need for pharmaceutical formulations comprising TCP-25 peptides with high efficacy, in particular high anti-bacterial efficacy and/or anti-inflammatory efficacy.

Interestingly, the present invention provides pharmaceutical formulations comprising TCP peptides, capable of retaining a significant amount of TCP peptides at the site of local application, which at the same time do not interfere negatively with the anti-inflammatory and anti-bacterial effect of the TCP peptides.

The action of TCP-25 and other TCP peptides involves structural transitions such as formation of a C-formed turn and a helical structure upon LPS-binding, and relies to some extent on the ability for both bacterial membrane and CD14 interactions. Unfortunately, some formulations induce structural changes in TCP-25, which results in loss of activity. Interestingly, the formulations provided by the present invention do not interfere with TCP peptide structure and supports TCP peptide functions.

The present invention also provides pharmaceutical formulations comprising TCP peptides having high stability. Interestingly, the invention shows that compositions comprising TCP peptides at high concentrations, are more stable. Such compositions are for example more resistant to denaturation. The invention shows that TCP peptides oligomerizes at high concentrations in a reversible manner. Without being bound by theory it is believed that the oligomerization may aid in stabilizing TCP peptides.

The present invention also provides pharmaceutical formulations comprising TCP peptides having high anti-bacterial efficacy. Interestingly, the invention shows that the anti-bacterial efficacy of TCP peptides may be significantly increased in the presence of EDTA.

Accordingly, it is an objective of the present invention to provide compositions suitable for containing the TCP-25 peptide and/or other TCP peptides. It is also an objective of the invention to provide stable compositions containing the TCP-25 peptide and/or other TCP peptides. It is also an objective of the invention to provide compositions containing the TCP-25 peptide and/or other TCP peptides with high anti-bacterial activity.

The means of accomplishing each of the above objectives as well as others will become apparent from the description of the invention, which follows hereafter.

The present invention discloses that a non-ionic hydrogel comprising TCP peptides provides a local delivery scaffold, which may mimic the endogenous actions of wound-derived host defense peptides (HDP) that are found in biological matrices such as fibrin. As further shown in the example section the present inventors show that the hydrogels comprising TCP peptides can act as a “dual-function” local therapeutic that targets both bacteria and the accompanying inflammatory response in experimental wound models. These therapeutic effects are however contingent upon the proper composition of the hydrogel, in particular that the hydrogel comprises a non-ionic polymer capable of forming a hydrogel. Such hydrogels are believed to provide TCP peptides with a local environment supporting the therapeutic effect. As shown herein formulations according to the invention have antibacterial activity.

In addition, it is shown that formulations according to the invention are capable of reducing inflammation.

Thus the present invention provides compositions comprising:
a) a compound comprising a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues,
b) a non-ionic polymer capable of forming a hydrogel when mixed with an aqueous solution, and
   an aqueous solution

Formulations comprising a non-ionic polymer capable of forming a hydrogel supports the antibacterial and/or anti-inflammatory activity of TCP peptides. Without wishing to be bound by theory this is contemplated to be connected with the action of TCP peptides involving structural transitions such as formation of a C-formed turn and a helical structure upon LPS-binding, and that it requires the ability for both bacterial membrane and CD14 interactions

It is also an aspect of the invention to provide compositions comprising:
a) a compound comprising a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues, and
b) EDTA and
c) an aqueous buffer,
   wherein the composition has a pH of at the most 7.

Formulations comprising EDTA supports the antibacterial activity of TCP peptides.

It is also an aspect of the invention to provide compositions comprising a compound comprising a peptide comprising or consisting of the amino acid sequence
X₁-X₂-X₃-X₄-X₅-X₆-W-X₈-X₉-X₁₀, wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues, and
wherein the concentration of the peptide of the composition is at least 0.01 wt %, preferably at least 0.08 wt %, such as at least 0.1 wt %, for example in the range of 0.08 to 3 wt %.

Such compositions in general have high stability, and the TCP peptides contained therein are in general more resistant to denaturation.

It is also an aspect of the present invention to provide products comprising the compositions of the invention.

In addition it is an aspect to provide the compositions of the invention for use in a method of treatment of treatment of a disorder in an individual in need thereof, wherein the composition is prepared for local administration.

### DESCRIPTION OF THE FIGURES

FIG. 1A-D show antibacterial and anti-endotoxic effects of TCP-25 in various formulations. FIG. 1A shows peptide activity and the release profile of TCP-25 formulations. The activity of TCP-25 in various formulations (HPC, CMC, and pluronic) was determined by evaluating the antimicrobial activity against E. coli, P. aeruginosa, and S. aureus using RDA. The bar chart illustrates measurements of the zones of clearance that were obtained. These correspond to the inhibitory effect of released peptide. Data are presented as the means (n=3). FIG. 1B shows a bar chart showing antimicrobial effects of TCP-25 in various formulations (HPC, CMC, and pluronic) as assessed by a viable count assay (VCA). E. coli, P. aeruginosa, and S. aureus were incubated with formulation substances with or without TCP-25. To quantify antimicrobial activity, appropriate dilutions of reaction mixtures were plated on TH broth agar followed by incubation overnight at 37° C. and the number of CFU was determined. Data are presented as the means (n=3). To investigate if TCP-25 formulations block endotoxin-induced pro-inflammatory responses, THP-1-XBlue™-CD14 cells were stimulated with E. coli LPS, in presence of various formulations (HPC, CMC, and pluronic) with and without TCP-25. The bar chart in FIG. 1C indicates NF-κB activation, as determined by measuring the production of SEAP. The values represent mean values (n=3). To assess cell viability of the TCP-25 formulations, an MTT assay was used. The bar chart in FIG. 1D shows the percentage of viable cells, as quantified using the MTT assay. Values are shown in comparison to the untreated live cells (100%, dotted line). Data are presented as the mean±SEM (n=3). P values were determined using a Kruskal-Wallis test followed by Dunn's post test. *P≤0.05; NS, not significant.

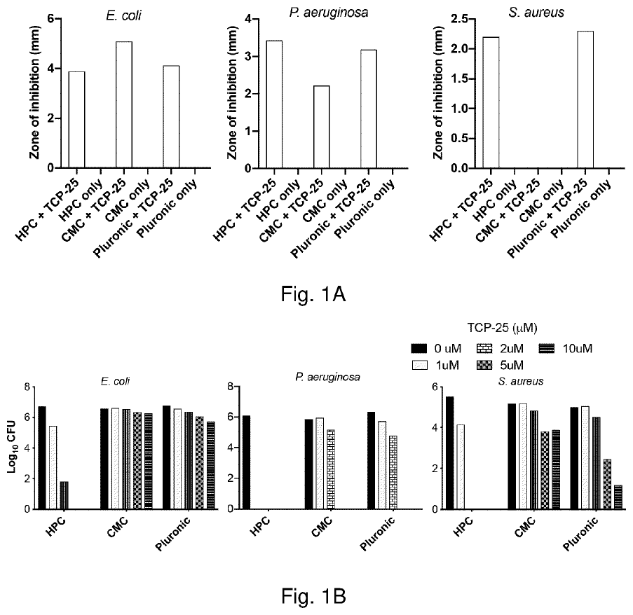

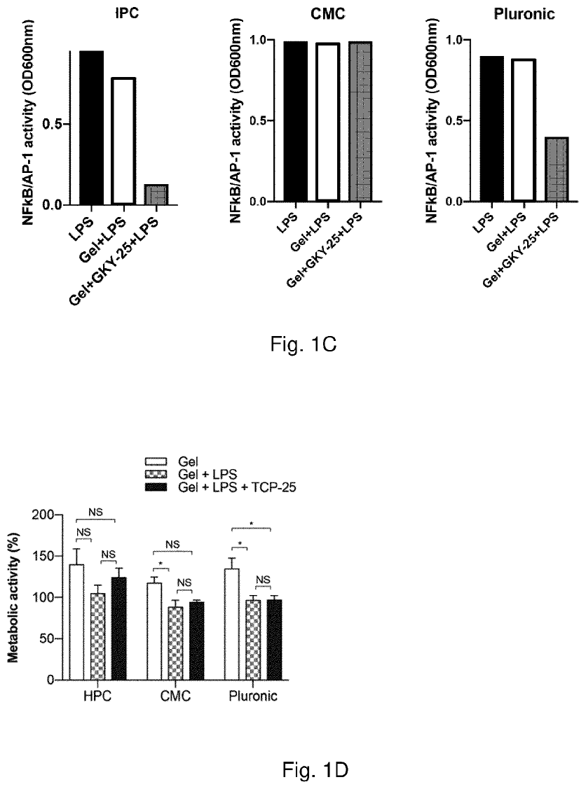

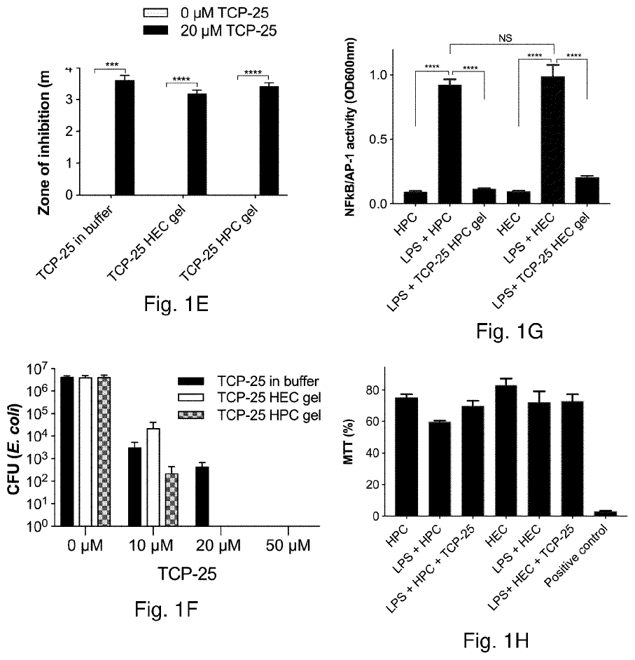

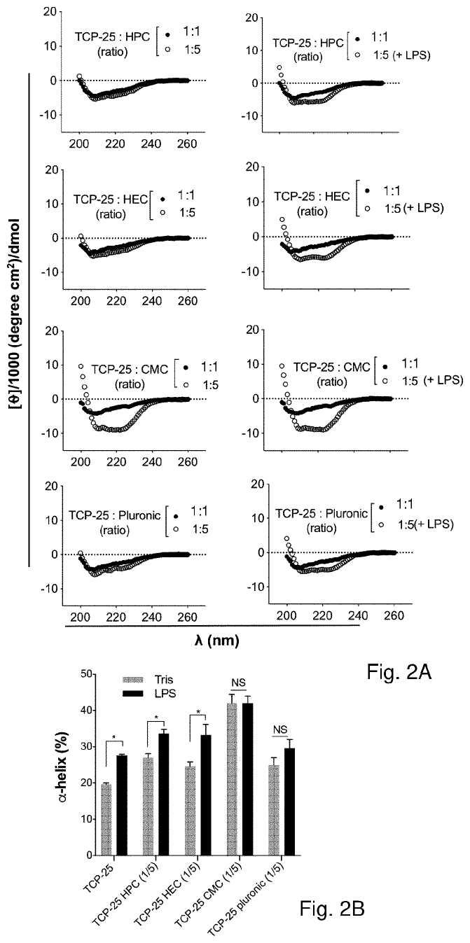

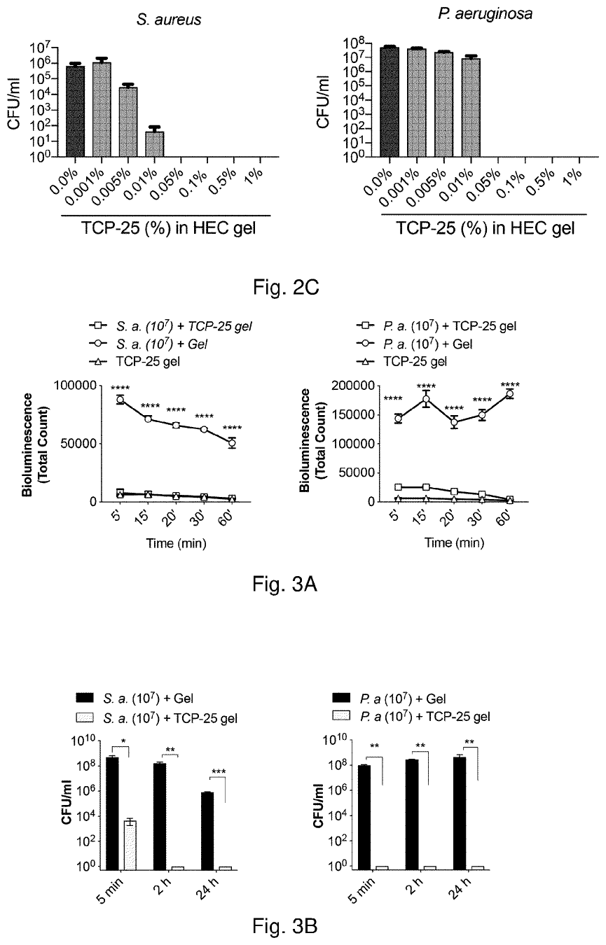

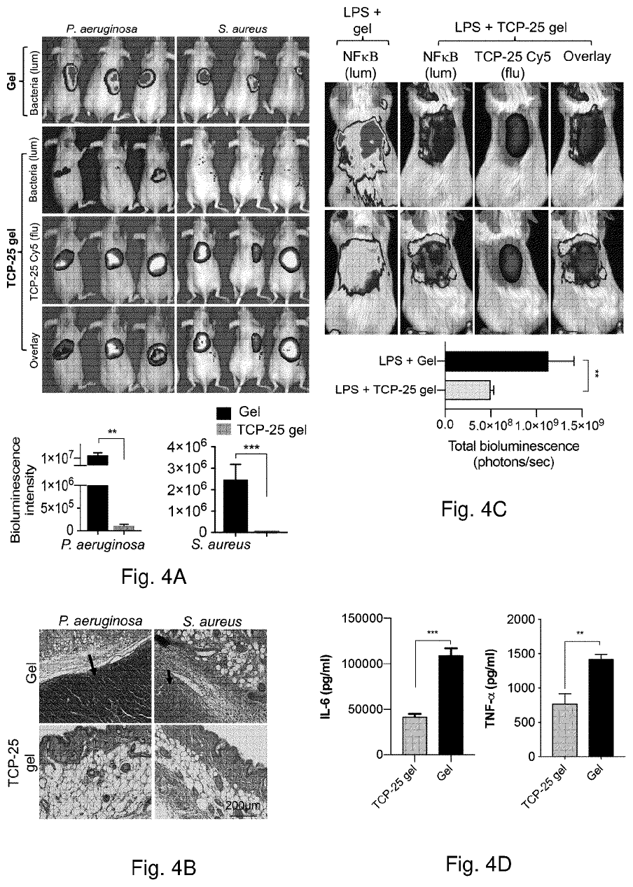

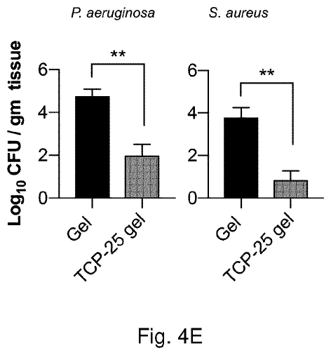

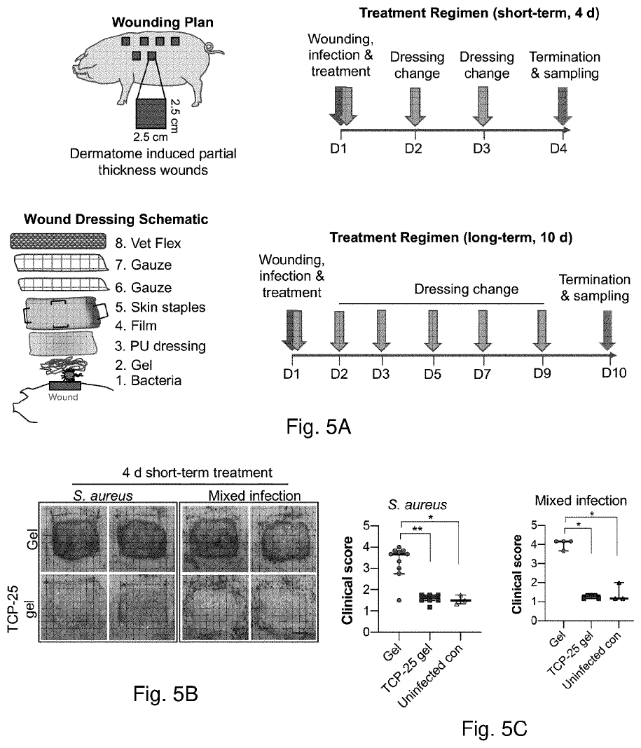

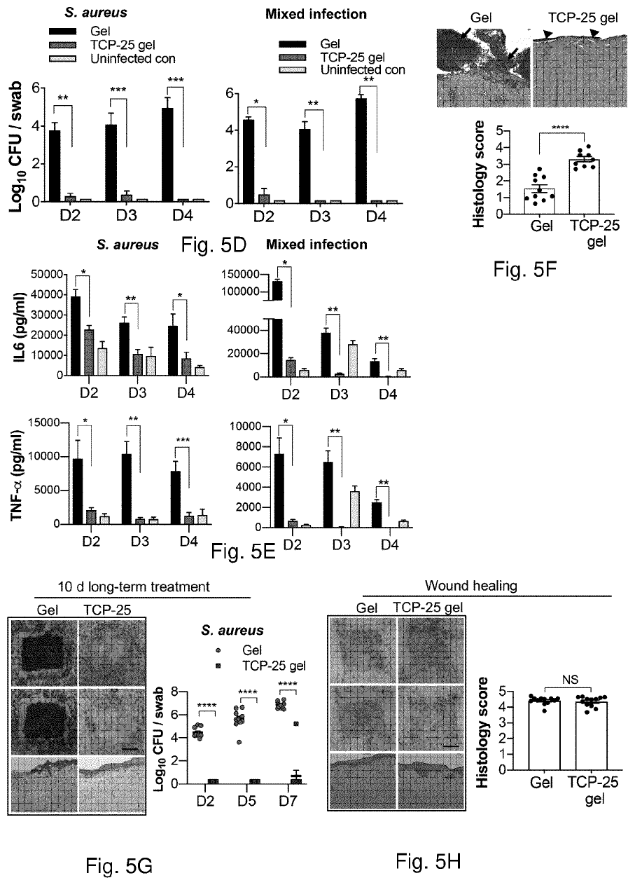

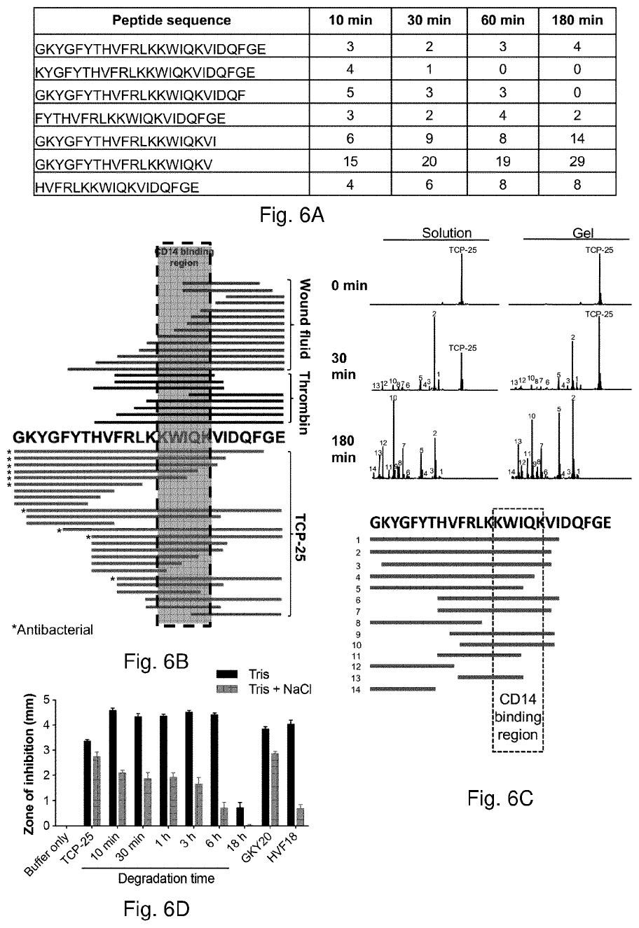

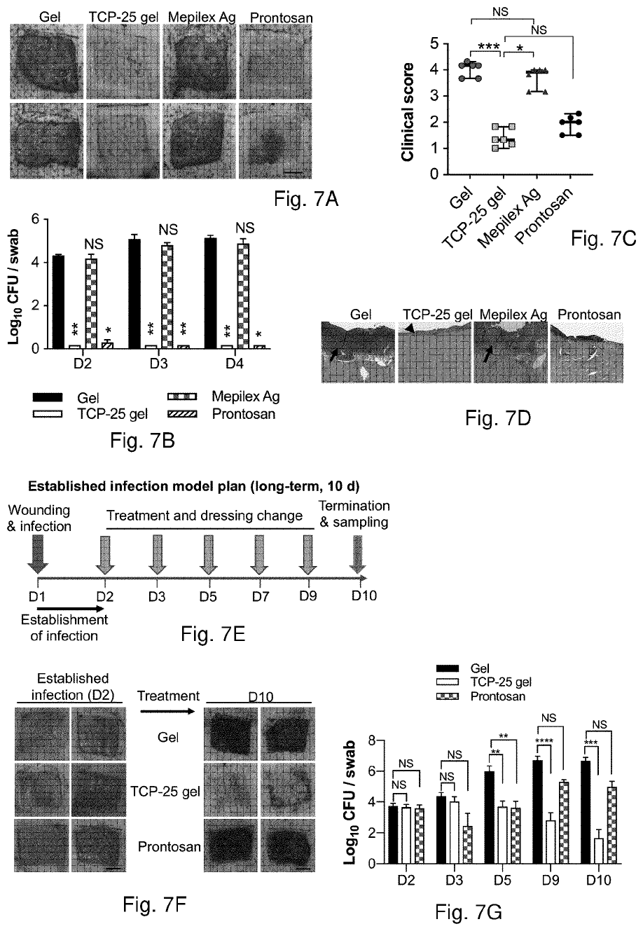

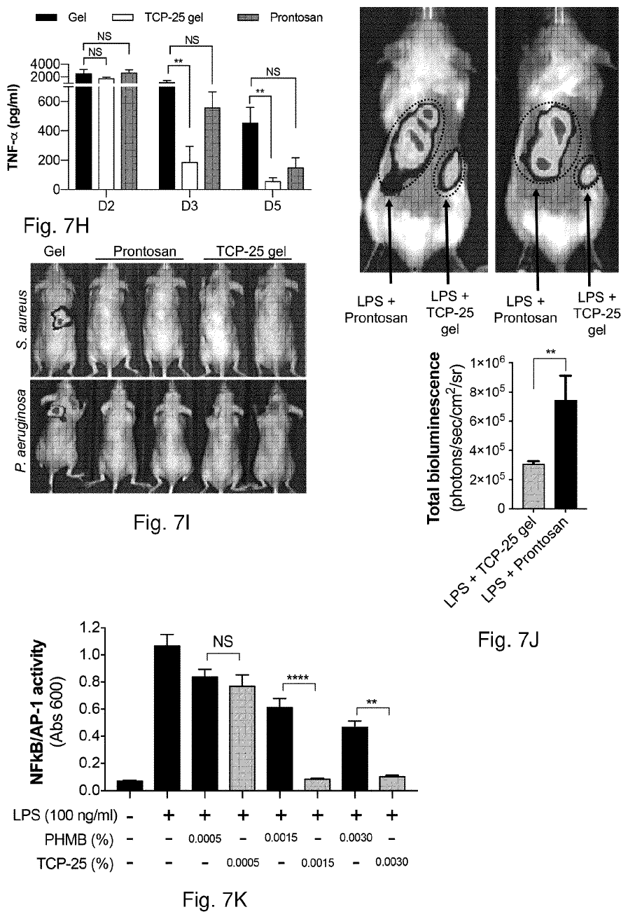

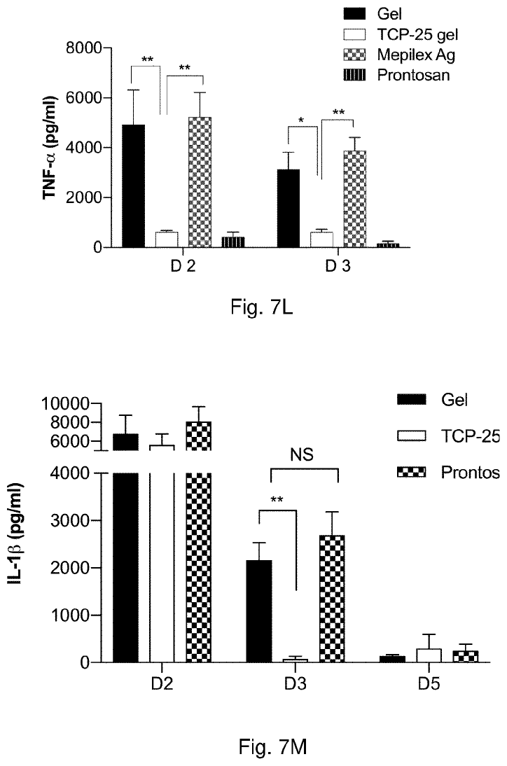

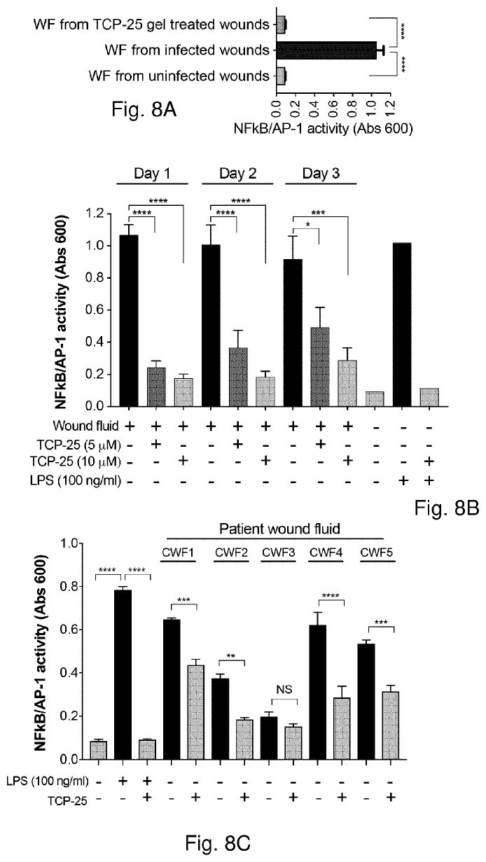

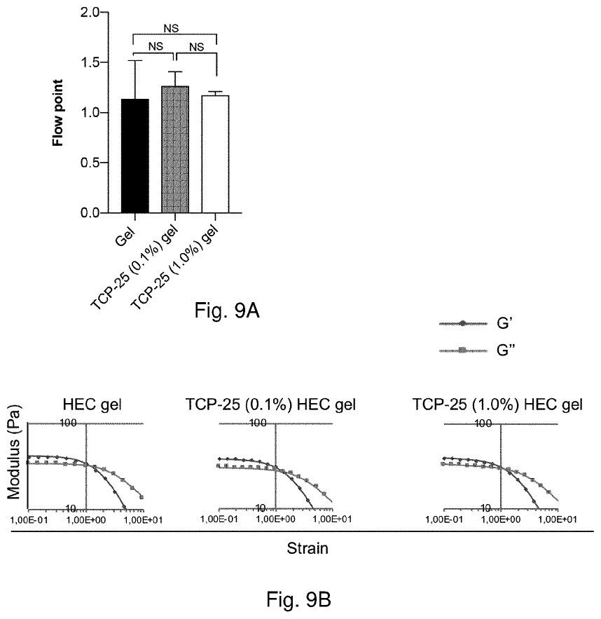

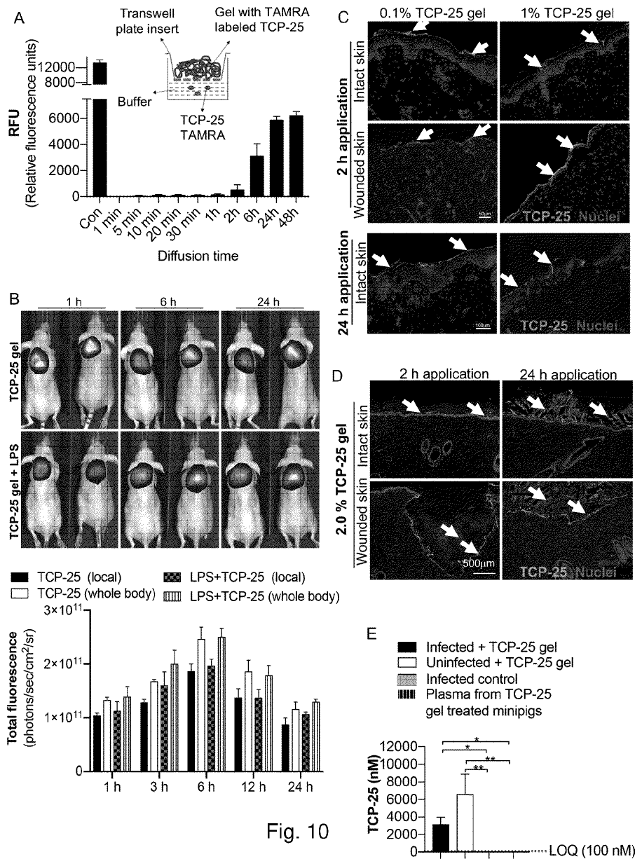

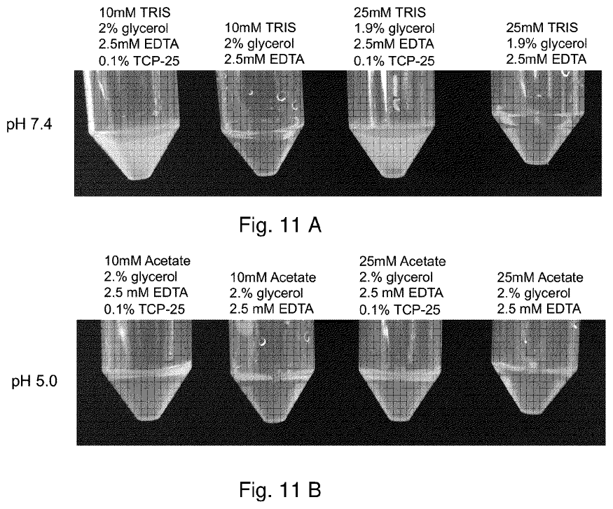

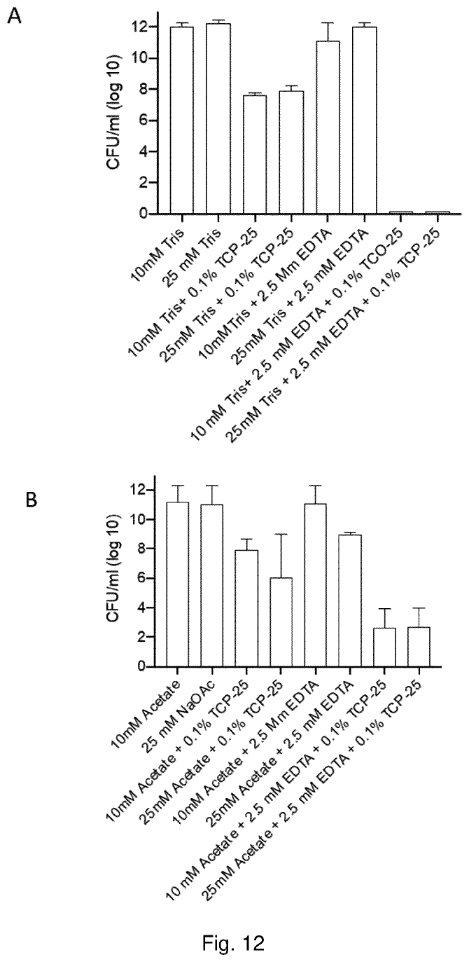

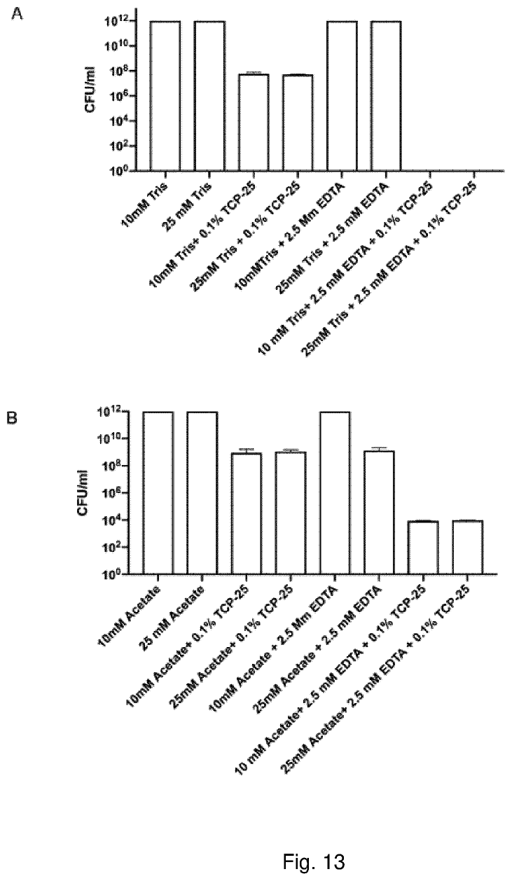

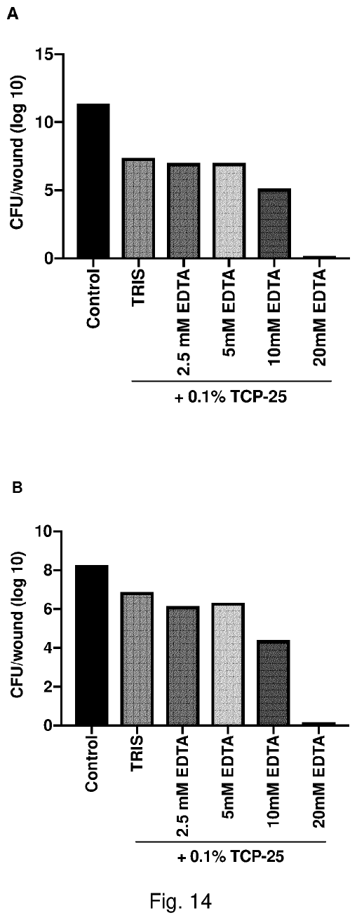

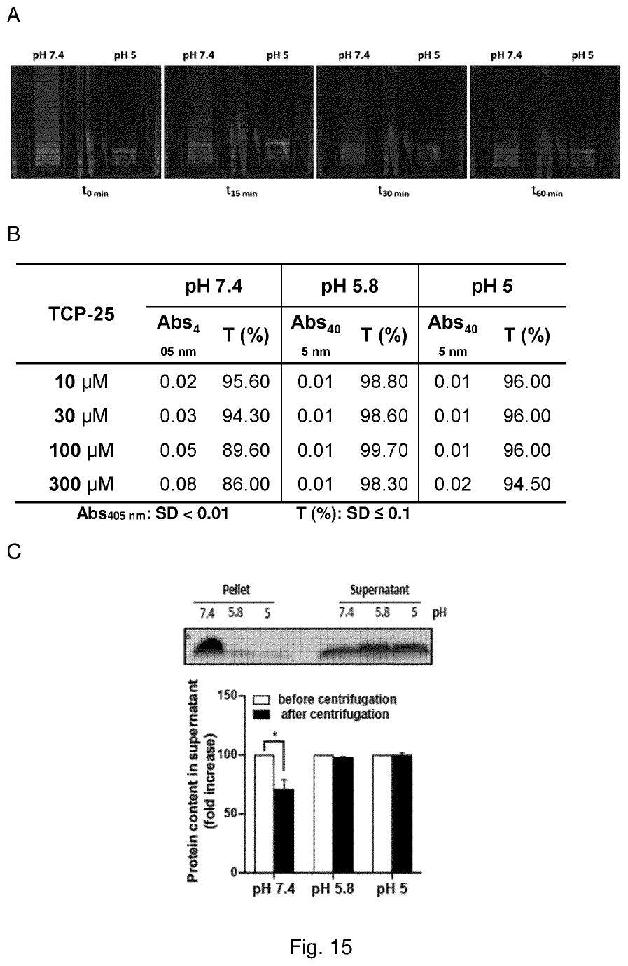

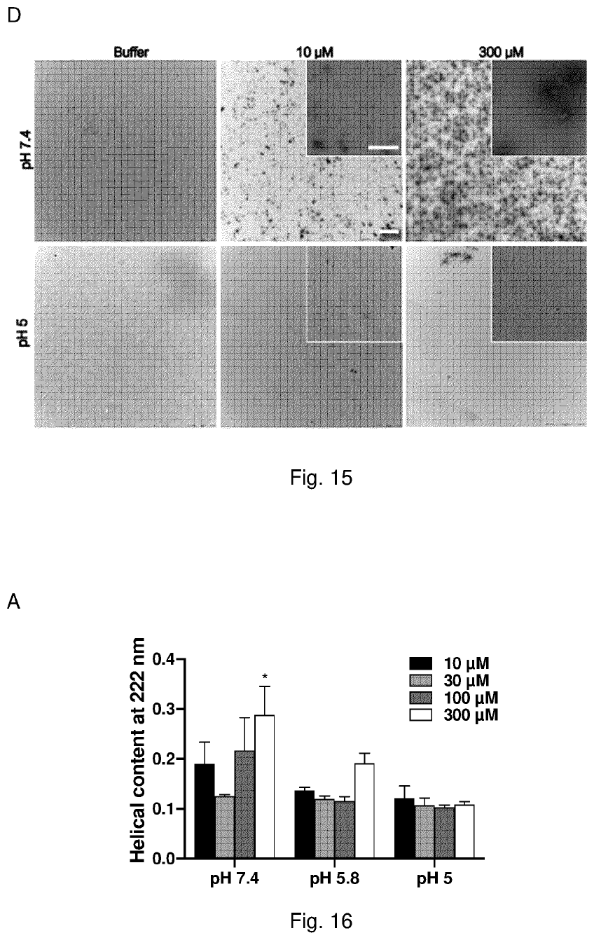

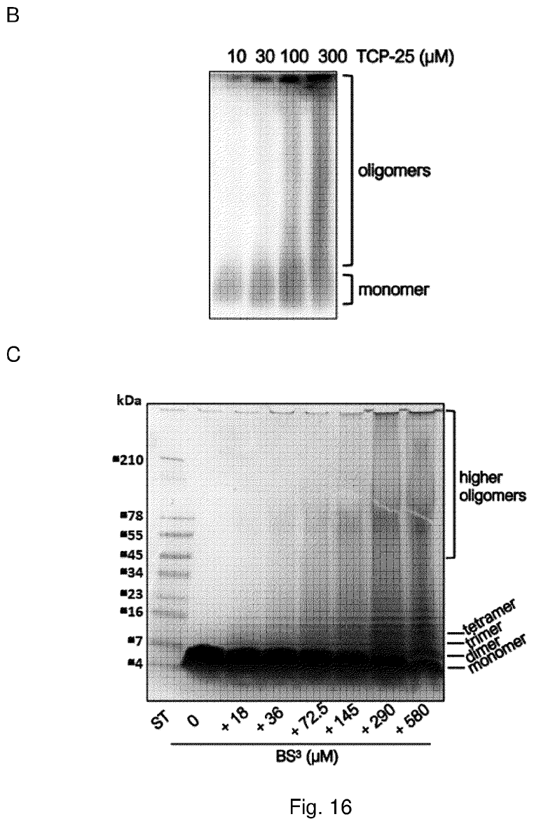

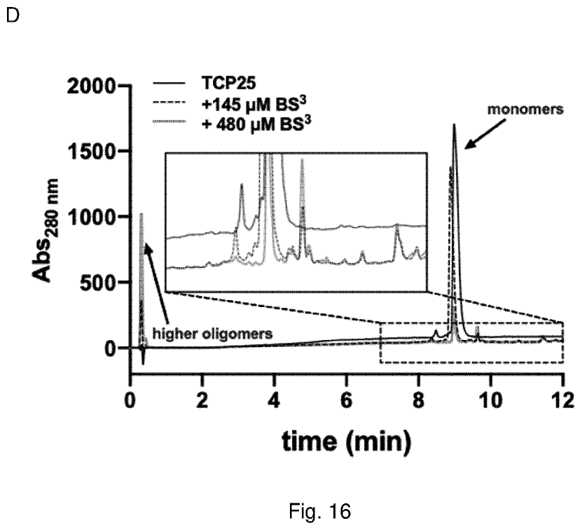

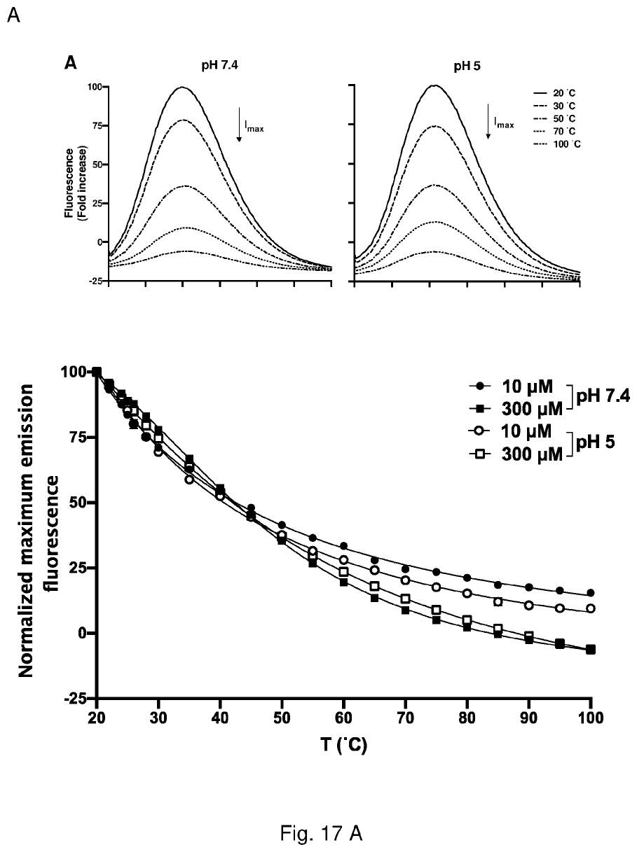

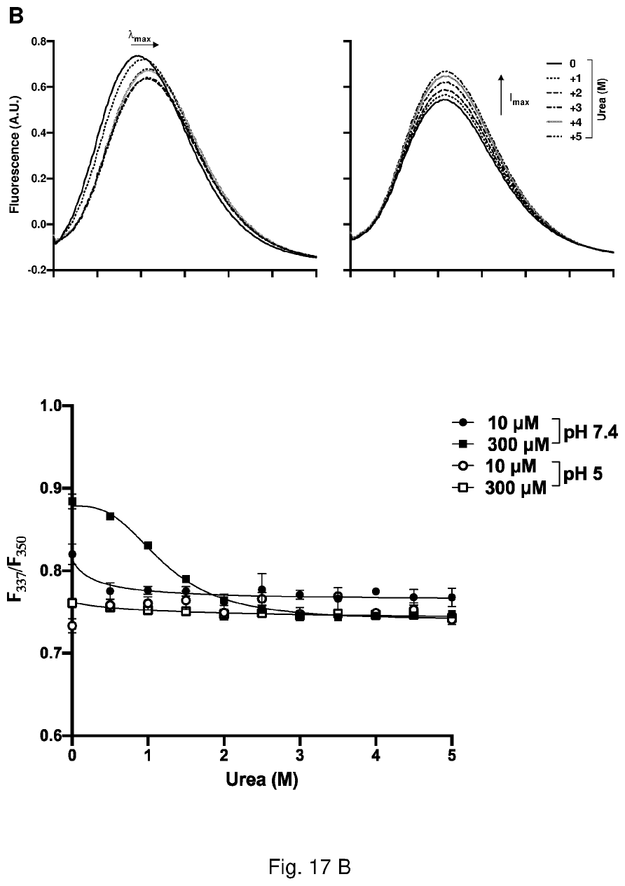

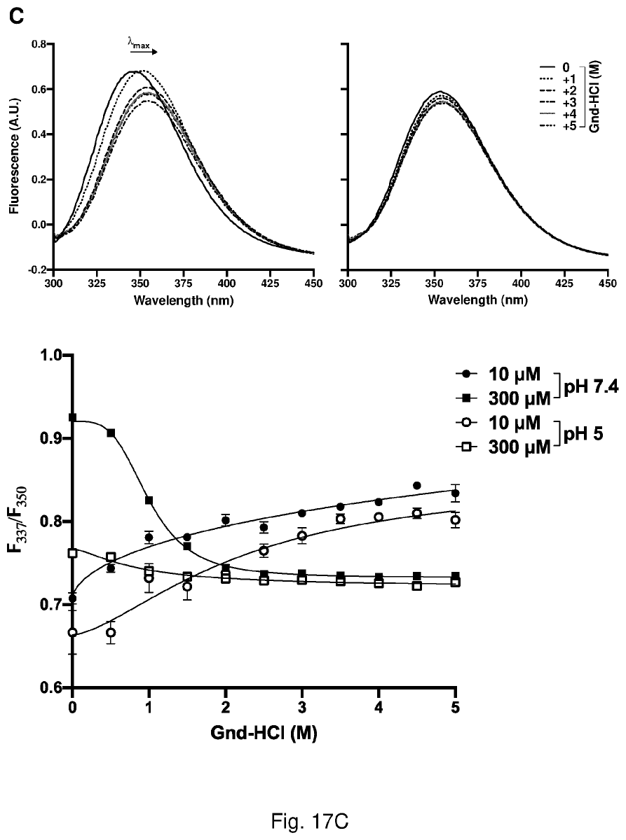

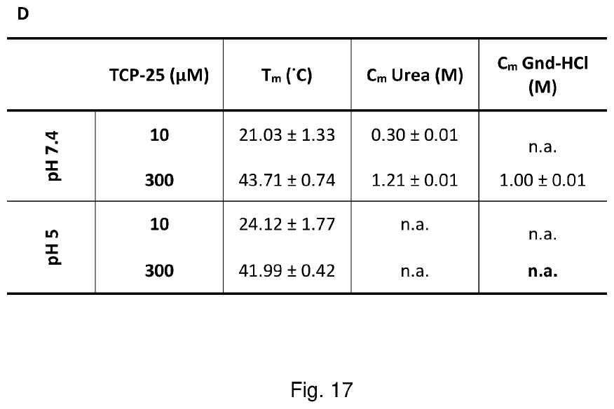

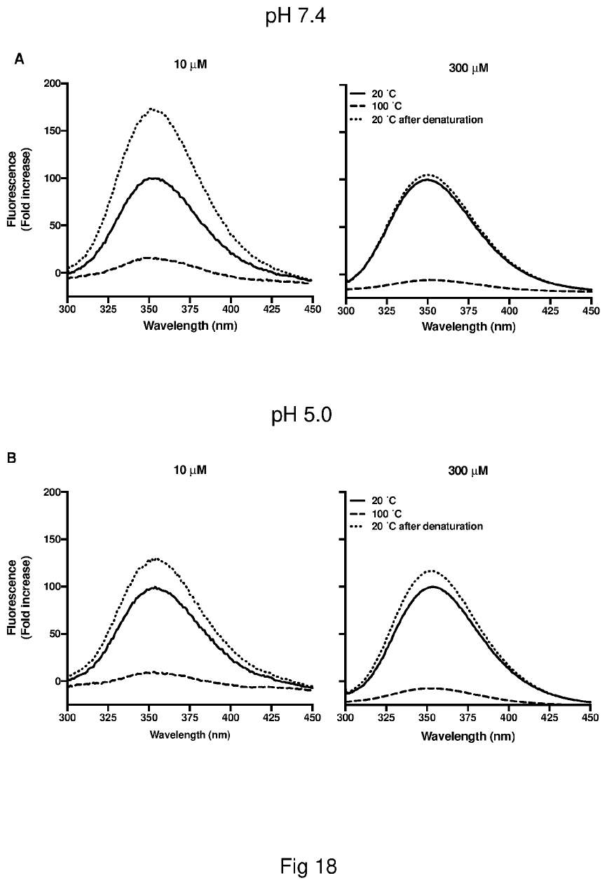

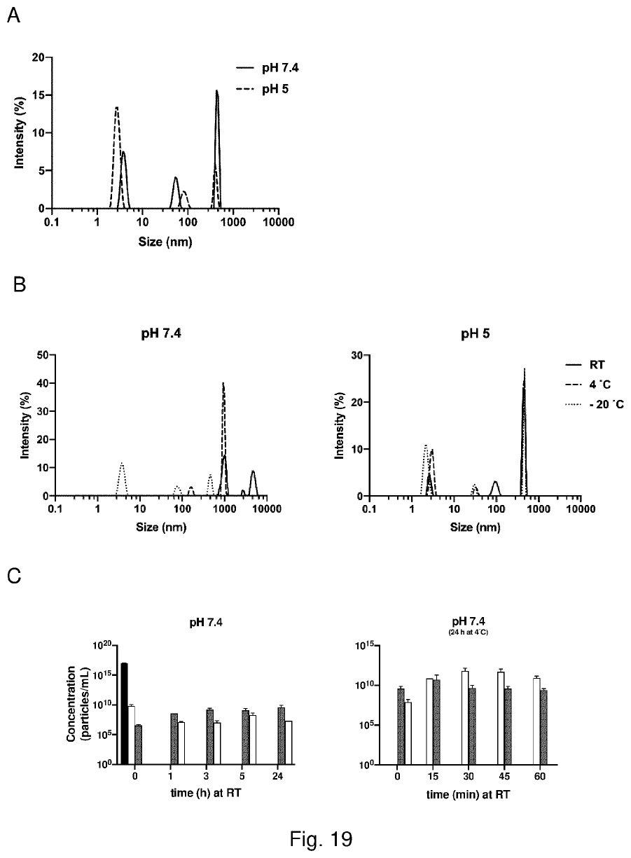

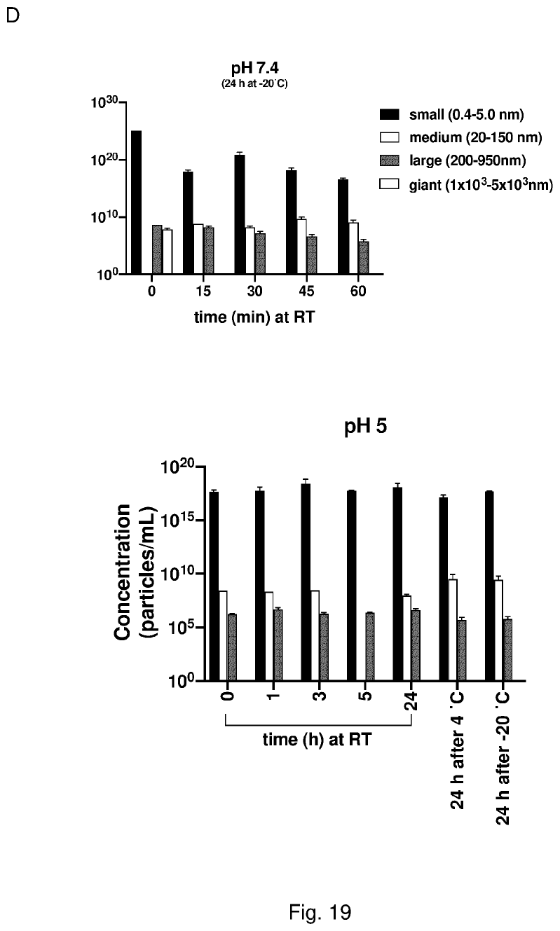

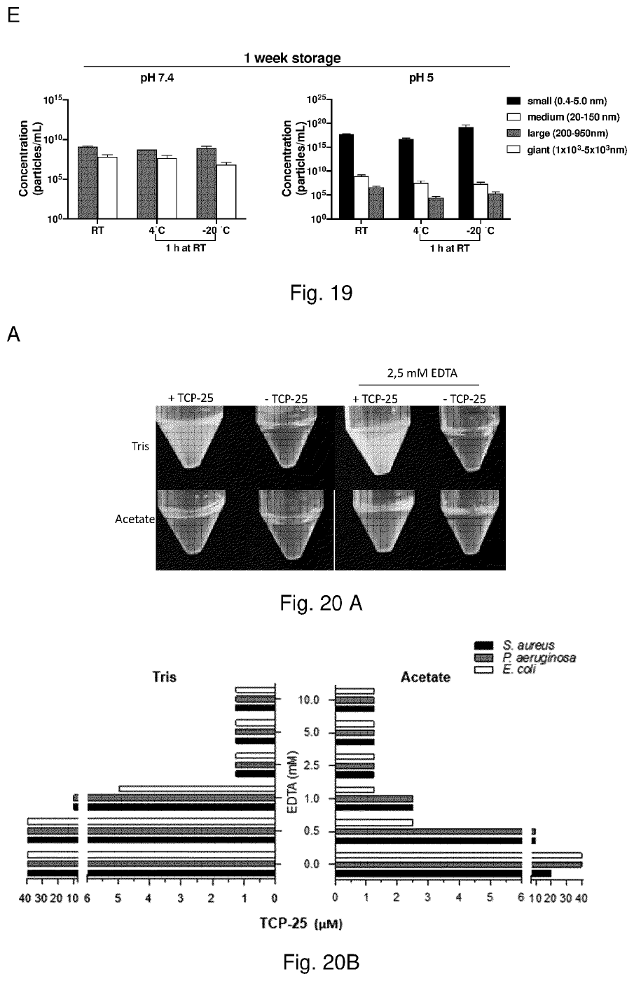

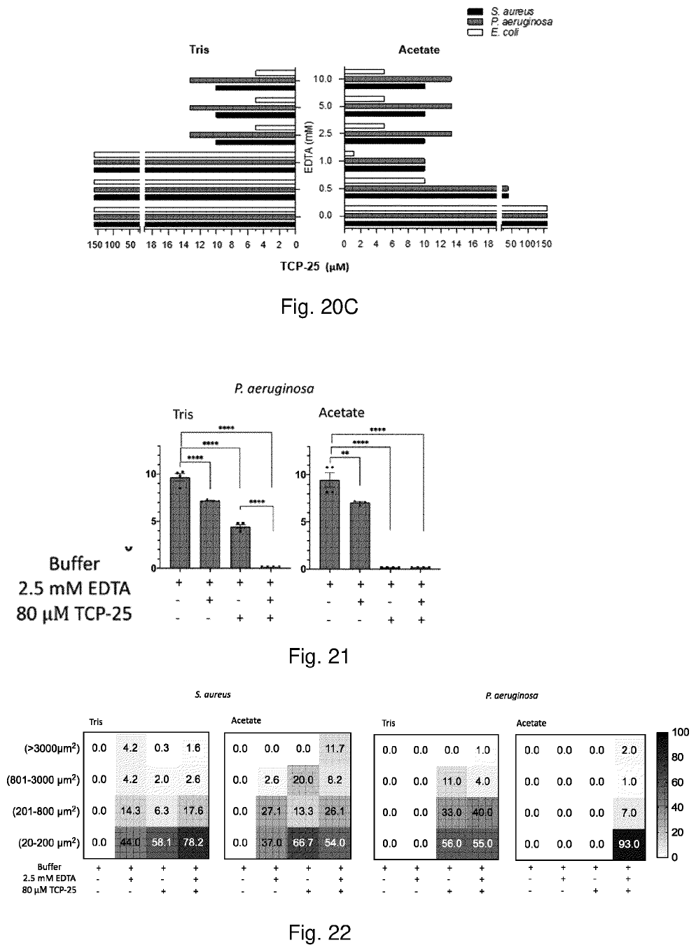

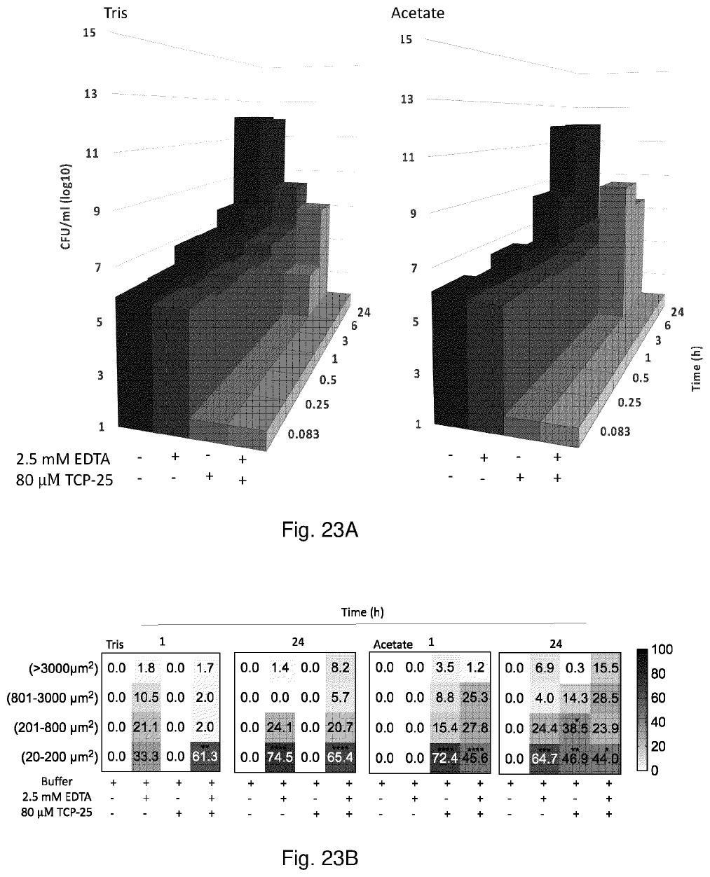

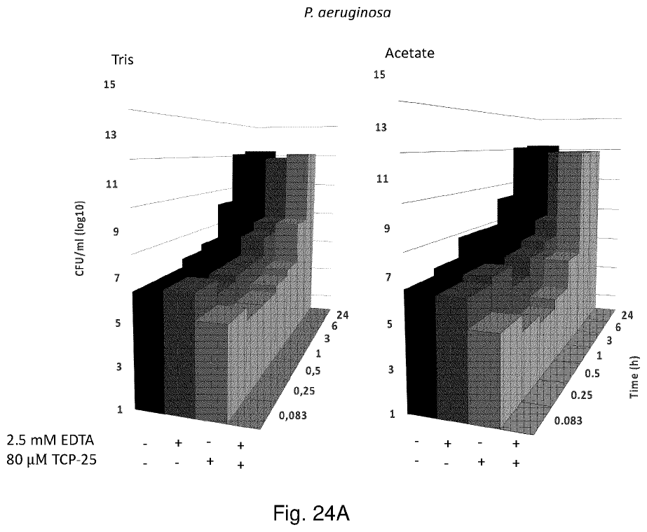

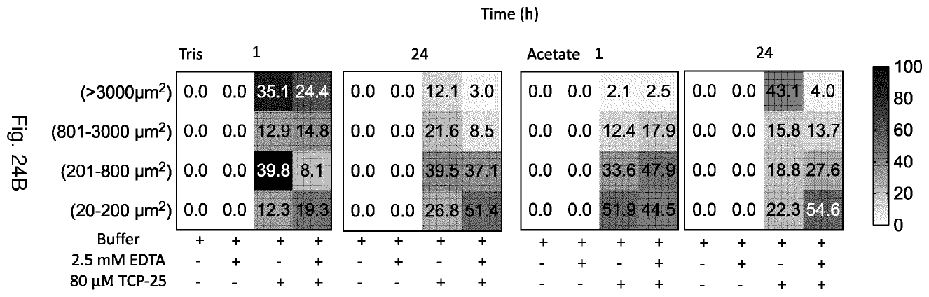

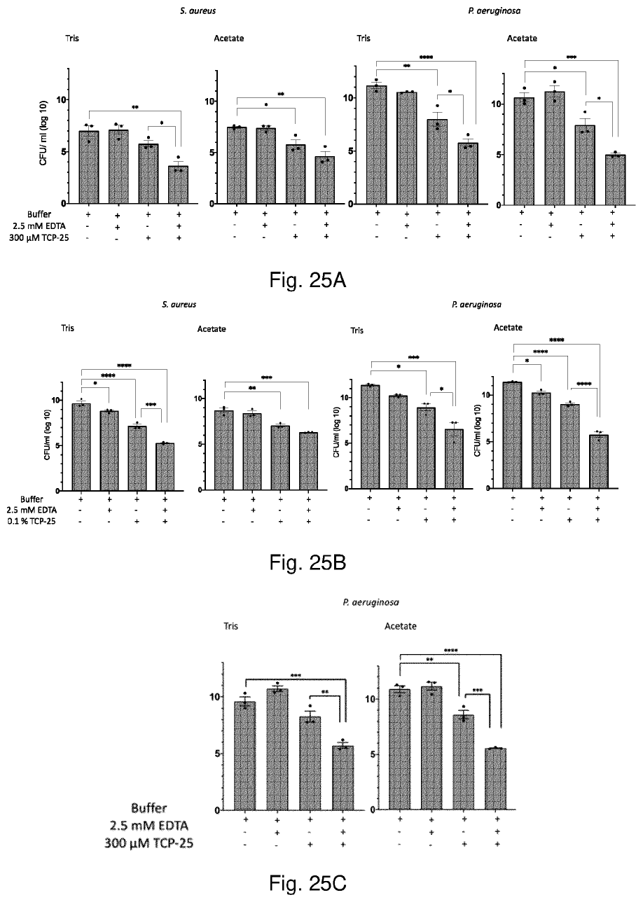

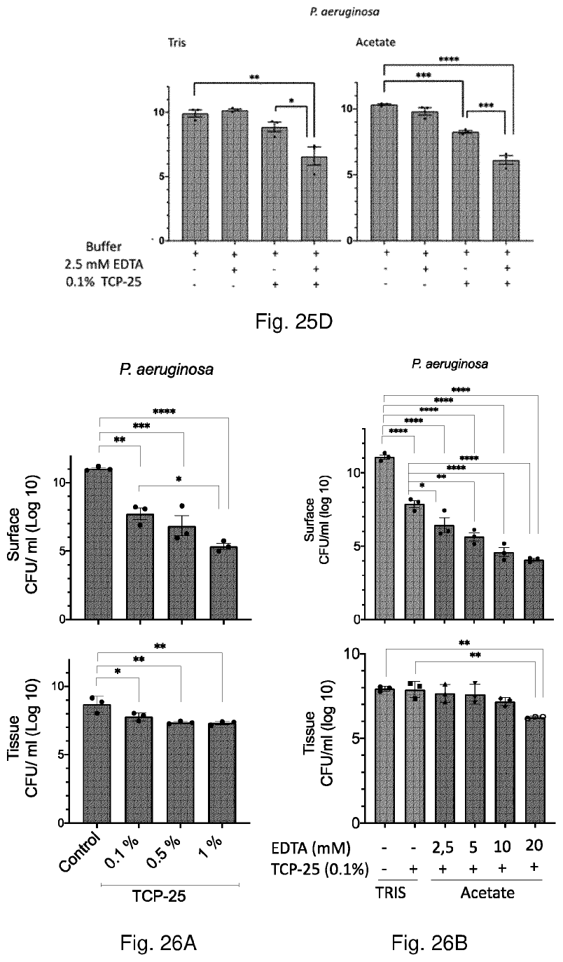

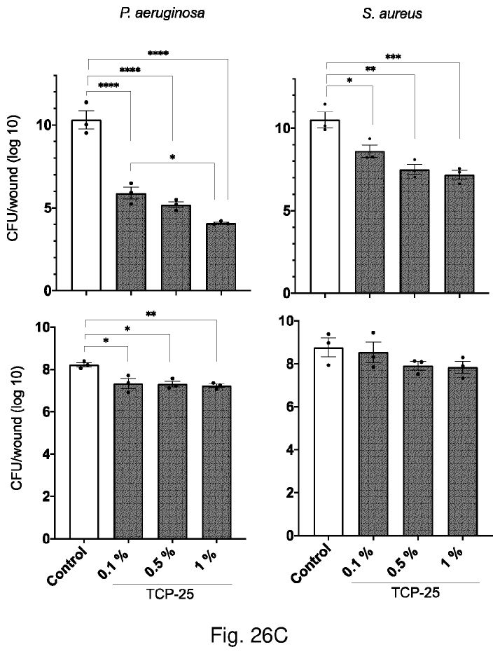

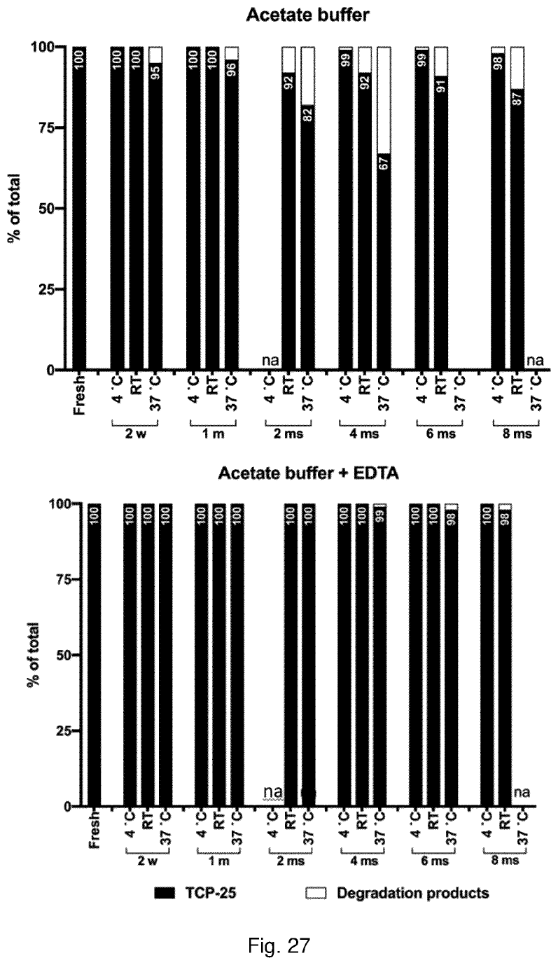

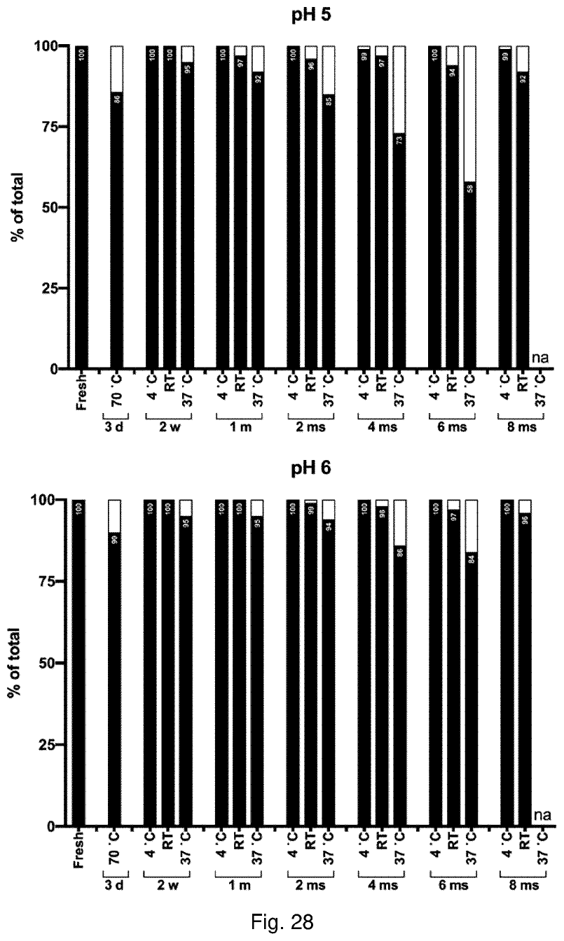

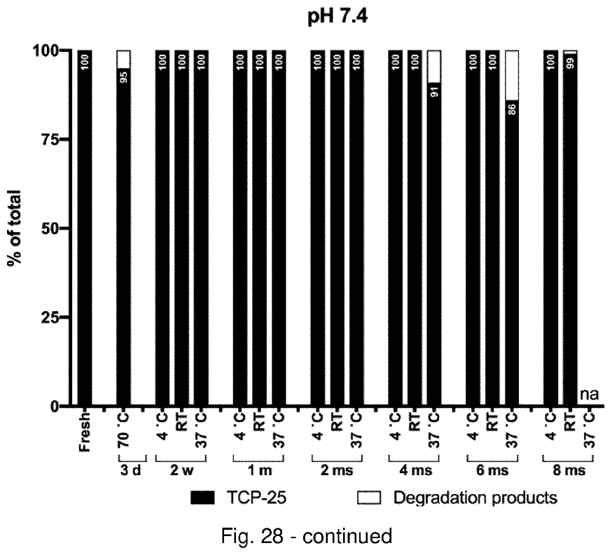

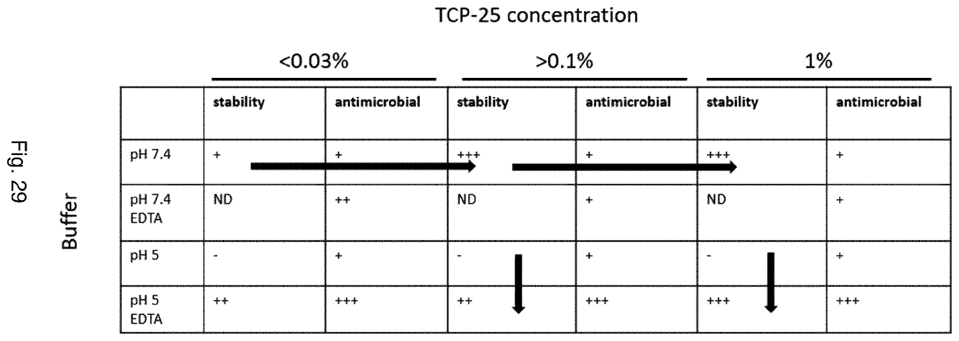

Further, FIG. 1E-H shows a comparison of TCP-25 formulation in HPC with the related polymer hydroxyethyl cellulose (HEC). Figure E shows peptide activity and release profile of TCP-25 in HPC and HEC. The release and activity of TCP-25 was determined by evaluating the antimicrobial activity against E. coli in RDA. The figure illustrates measurements of the zones of clearance obtained. Data are presented as the mean±SEM (n=6). P values were determined using a Mann-Whitney U test. FIG. 1F shows VCA showing antimicrobial effects of the TCP-25 formulation in HPC and in HEC. E. coli was incubated with the formulation gels with or without TCP-25. To quantify antimicrobial activity, appropriate dilutions of reaction mixtures were plated on TH broth agar followed by incubation overnight at 37° C. and the number of CFU was determined. Data are presented as the mean±SEM (n=3). FIG. 1G shows a comparison of antiendotoxic effects of TCP-25 formulations in HPC and HEC, wherein THP-1-XBlue™-CD14 cells were stimulated with E. coli LPS in presence of formulations with and without TCP-25. The bar chart indicates NF-κB activation as determined by measuring the production of SEAP. Data are presented as the mean±SEM (n=6). P values were determined using a one-way ANOVA with Tukey's post test. FIG. 1H shows simultaneous analyses of toxic effects of formulation components alone and in combination with TCP-25 were performed. The histogram shows the percentage of viable cells as quantified using the MTT assay. Lysed cells were used as a positive control. Values are shown in comparison to the untreated live cells (100%). Data are presented as the mean±SEM (n=3). ***P≤0.001; ****P≤0.0001; NS, not-significant.

FIG. 2A-C show secondary structural changes of TCP-25 determined by CD spectroscopy. FIG. 2A shows CD spectra of TCP-25, measured after incubation with Tris buffer, LPS, HPC, HEC, CMC, or pluronic (TCP-25-to-polymer ratios of 1:1 and 1:5). FIG. 2B shows α-helical content of TCP-25 calculated from molar ellipsometry at 222 nm in the presence of Tris buffer, LPS, and polymers (ratio of 1:5). Data are presented as the mean±SEM (n=3). P values were determined using a Mann-Whitney U test. *P s 0.05; NS, not significant. FIG. 2C shows VCA showing the antimicrobial effect of varying concentrations of TCP-25 in the HEC gel formulation. S. aureus and P. aeruginosa were incubated with hydrogels with or without TCP-25. To quantify antimicrobial activity, appropriate dilutions of reaction mixtures were plated on TH broth agar followed by incubation overnight at 37° C. and the number of CFU was determined. Data are presented as the mean±SEM (n=3).

FIG. 3A-B show In vitro antibacterial effects of TCP-25 formulated in a HEC gel (TCP-25 Gel #1). FIG. 3A shows bacterial bioluminescence measurement after treatment with TCP-25 gel #1. Bioluminescent S. aureus or P. aeruginosa (10⁷/mL CFU) were treated with TCP-25 formulation. Bioluminescence emitted from bacteria was measured using a luminescence plate reader. Line chart shows total bioluminescence count at the indicated time points. Data are presented as the mean±SEM (n=3). P values were determined using a two-way ANOVA with Tukey's post test. FIG. 3B shows a VCA showing antimicrobial effects of TCP-25 HEC formulation against S. aureus or P. aeruginosa. Data are presented as the mean±SEM (n=3). P values were determined using a Mann-Whitney U test. P values were determined using an unpaired t tests. Comparisons were made with respective gel controls. *P≤0.05; **P≤0.01; ***P≤0.001; ****P≤0.0001.

FIG. 4A-E show anti-bacterial and anti-inflammatory effects of TCP-25 gel #1 formulation in a mouse model of subcutaneous infection and inflammation. FIG. 4A shows In vivo infection imaging by IVIS in the mouse model of subcutaneous infection. Control HEC gel and TCP-25 gel #1 was deposited subcutaneously in the dorsum of SKH1 mice after inoculation with 10⁶ CFU of bioluminescent P. aeruginosa or S. aureus bacteria. To visualize in vivo drug localization, TCP-25 used in the formulation was spiked with Cy5 labeled TCP-25. At different time points, bacterial bioluminescence intensity and TCP-25 Cy5 fluorescence were non-invasively analyzed using the IVIS bioimaging system. Representative images show bacterial luminescence (lum) and TCP-25 Cy5 fluorescence (flu) at 6 h post infection. The bar chart shows measured bioluminescence intensity emitted by the bacteria at 6 h post infection. Data are presented as the mean±SEM (n=7 mice for gel group and 7 mice for TCP-25 gel #1 group for each bacterial infection). P values were determined using Mann-Whitney U test. FIG. 4B shows representative images of H&E staining of mouse skin tissue from the site of gel deposition. Arrows show tissue destruction and the hyper-inflammatory condition of the tissue. FIG. 4C shows In vivo inflammation imaging by IVIS in NF-κB reporter mice. LPS in HEC gel or in TCP-25 HEC formulation was subcutaneously deposited on the back of transgenic BALB/c Tg(NF-κB-RE-luc)-Xen reporter mice. In vivo bioimaging of NF-κB reporter gene expression was performed using the IVIS Spectrum system. To image in vivo drug localization, TCP-25 was spiked with Cy5-labeled TCP-25. Representative images show bioluminescence (lum) and TCP-25 Cy5 fluorescence (flu) at 6 h. A bar chart shows measured light intensity emitted from these reporter mice. Data are presented as the mean±SEM (n=7 mice for gel group, 5 mice for TCP-25 gel #1 group). P values were determined using Mann-Whitney U test. FIG. 4D shows cytokine analysis from the wound fluid extracted from implanted PU discs. Data are presented as the mean±SEM (n=4 gel, n=4 TCP-gel #1). P values were determined using Mann-Whitney U test. **P≤0.01; ***P≤0.001.

FIG. 4E further shows the microbiological analysis of tissue 24 h post infection. Data are presented as the mean±SEM (n=7 mice for gel and 7 mice for TCP-25 gel #1). P values were determined using Mann-Whitney U test. **P≤0.01.

FIG. 5A-H show effects of TCP-25 gel in a porcine partial thickness wound model. FIG. 5A illustrates the wounding plan in minipigs. Twelve partial thickness wounds, six on each side, were created using an electric dermatome on the backs of Gottingen minipigs and infected with S. aureus. Each wound was infected with 10⁷ CFU of S. aureus. The figure also illustrates the wound dressing plan. Briefly, after infection and application of gel, wounds were covered with a primary polyurethane dressing followed by a transparent breathable fixation dressing. For better fixation, dressings were then secured with skin staples. The wound area was then covered with two layers of sterile cotton gauze and secured with adhesive tape. Finally, a layer of flexible self-adhesive bandage was used to support and protect dressings underneath. Additionally, the figures describe two therapeutic approaches, short-term and long-term, that were used for the minipig study. FIG. 5B shows representative photographic images of minipig wounds after the short-term treatment regimen. Wounds with either S. aureus or having a mixed infection (S. aureus and superinfection with P. aeruginosa) were treated every day with gel with or without TCP-25. Uninfected control wounds were treated with gel without TCP-25 (Scale bar, 1 cm). FIG. 5C shows clinical scoring of wounds after the short-term treatment regimen. Data are presented as the medians with 95% confidence intervals (For S. aureus infection group, n=10 wounds for gel, n=9 wounds for TCP-25 gel, and n=3 wounds for uninfected controls from 4 pigs. For mixed infection group, n=4 wounds for gel, n=5 wounds for TCP-25 gel, and n=3 wounds for uninfected controls from 2 pigs). P values were determined using a Kruskal-Wallis test followed by Dunn's post test. FIG. 5D shows microbiological analysis of wounds from days 2, 3, and 4. Data are presented as the mean±SEM (For S. aureus infection group, n=10 wounds for gel, n=9 wounds for TCP-25 gel, and n=3 wounds for uninfected controls from 4 pigs. For mixed infection group, n=4 wounds for gel, n=5 wounds for TCP-25 gel, and n=3 wounds for uninfected controls from 2 pigs). P values were determined using a Kruskal-Wallis test followed by Dunn's post test. FIG. 5E shows analysis of wound fluid cytokines collected on days 2, 3, and 4. Data are presented as the mean±SEM (For the S. aureus infection group, n=8-10 wounds for gel, n=7-9 wounds for TCP-25 gel, and n=3 wounds for uninfected controls from 4 pigs. For the mixed infection group, n=4 wounds for gel, n=5 wounds for TCP-25 gel, and n=3 wounds for uninfected controls from 2 pigs). P values were determined using a Kruskal-Wallis test followed by Dunn's post test. FIG. 5F shows representative images showing H&E staining of wound biopsies after 4 days of treatment. Arrows show severe tissue destruction and hyper-inflammatory condition of the wound. Arrowheads show wound re-epithelization. The bar chart shows histological analysis of wound tissues. Data are presented as the mean±SEM (n=12 wounds for gel, n=12 wounds for TCP-25 gel). P values were determined using a Mann-Whitney U test. FIG. 5G shows representative photographic images of minipig wounds after the long-term treatment regimen. Wounds were infected with S. aureus and treated on days 1, 2, 3, 5, 7, and 9 with TCP-25 gel. In the lower panel, images show H&E staining of wound biopsies. Dot plot shows microbiological analysis of wounds from days 2, 5, and 7. Data are presented as the mean±SEM (n=10 wounds for gel, n=10 wounds for TCP-25 gel from 4 pigs). P values were determined using a Mann-Whitney U test. FIG. 5H shows the effect of TCP-25 gel treatment on minipig wound healing (on non-infected wounds). Partial thickness wounds were created on minipigs and treated with TCP-25 gel. Representative photographic images of wounds and H&E-stained wound biopsies are shown. The bar chart shows histological analysis of the wound tissues. Data are presented as the mean±SEM (n=10 wounds for gel, n=9 wounds for TCP-25 gel from 4 pigs). P values were determined using Mann-Whitney U test. *P≤0.05; **P≤0.01; ***P≤0.001; ****P≤0.0001; NS, not-significant.

FIG. 6A-D show degradation of TCP-25 by human neutrophil elastase in vitro and comparison with proteolytic thrombin fragments generated in vitro and in vivo. FIG. 6A shows the digestion pattern of TCP-25 after treatment with HNE. Digestions with the enzyme were performed for different time periods and analyzed by mass spectrometry. The table shows the sequences of major peptides and the number of successful identifications by mass spectrometry at 10, 30, 60, and 180 minutes. FIG. 6B shows a graphical representation of major peptides obtained after digestion and comparison with peptides found after digestion of thrombin, and those detected in wounds in vivo. *Peptides reported to show antibacterial effects. FIG. 6C shows representative high-resolution MALDI mass spectra of HNE digested TCP-25. The same peptide fragments were detected in the buffer solution and the gel. After 180 min, no intact TCP-25 could be detected from the solution or gel sample. Identified peptide sequences are shown in the lower panel. FIG. 6D shows the release and activity of TCP-25 degradation products was determined by evaluating the antimicrobial activity against E. coli by RDA in 10 mM Tris, pH 7.4 with or without 0.15 M NaCl. The bar chart illustrates measurements of the zones of clearance obtained. Data are presented as the mean±SEM (n=3).

FIG. 7A-M shows a comparison of TCP-25 gel with wound treatment benchmarks. TCP-25 gel was compared with Mepilex Ag and Prontosan, two current standard benchmarks in wound care. FIG. 7A shows representative photographic images of minipig wounds after the short-term treatment regimen. Wounds were infected with 10⁷ CFU of S. aureus and treated once daily with TCP-25 gel, Mepilex Ag or Prontosan. FIG. 7B shows a microbiological analysis of wounds from days 2, 3, and 4. Swab samples were collected from wounds and appropriate dilutions were plated on TH broth agar and the number of CFU was determined. Data are presented as the mean±SEM (n=6 wounds for gel, n=6 wounds for TCP-25 gel, n=6 wounds for Mepilex Ag, n=6 wounds for Prontosan from 3 pigs). Comparisons are shown against ‘Gel’ group and P values were determined using a Kruskal-Wallis test followed by Dunn's post test. FIG. 7C shows clinical scoring of wounds after the short-term treatment regimen. Data are presented as the medians with 95% confidence intervals (n=6 wounds for gel, n=6 wounds for TCP-25 gel, n=6 wounds for Mepilex Ag, n=6 wounds for Prontosan from 3 pigs). P values were determined using a Kruskal-Wallis test followed by Dunn's post test. FIG. 7D shows representative images showing H&E staining of wound biopsies. Arrows show severe tissue destruction and inflammatory infiltrates in the wound. Arrowheads indicate areas of re-epithelization of the wound. FIG. 7E illustrates established infection model experimental plan in minipigs. FIG. 7F shows representative photographic images of minipig wounds at days 2 and 10 of established infection treatment regimen. Wounds were infected with S. aureus and after establishment of infection, treated on days 2, 3, 5, 7 and 9 with control gel, TCP-25 gel, or Prontosan (scale bar, 1 cm). FIG. 7G shows a microbiological analysis of wounds (from established infection model) from days 2, 3, 5, 9 and 10. Data are presented as the mean±SEM (n=7 wounds for gel, n=7 wounds for TCP-25 gel, and n=7 wounds for Prontosan from 2 pigs). P values were determined using a Kruskal-Wallis test followed by Dunn's post test. FIG. 7H shows an analysis of TNF-α in wound fluid collected on days 2, 3, and 5. Data are presented as the mean±SEM (n=7 wounds for gel, n=7 wounds for TCP-25 gel, and n=7 wounds for Prontosan from 2 pigs). P values were determined using a Kruskal-Wallis test followed by Dunn's post test. FIG. 7I shows in vivo infection imaging by IVIS in a mouse model of subcutaneous infection. TCP-25 gel formulations were deposited subcutaneously on the dorsum of SKH1 mice after adding bioluminescent S. aureus or P. aeruginosa bacteria. At different time points, bacterial bioluminescence intensity was non-invasively analyzed in the IVIS bioimaging system. Representative images show bacterial luminescence at 6 h post infection (n=6 for each group). FIG. 7J shows in vivo inflammation imaging by IVIS in NF-κB reporter mice. Prontosan or TCP-25 gel were mixed with LPS and subcutaneously deposited on the left and right side, respectively, on the back of transgenic BALB/c Tg(NF-κB-RE-luc)-Xen reporter mice. In vivo imaging of NF-κB reporter gene expression was achieved using an IVIS Spectrum bioimaging system. Representative images show bioluminescence 6 h after subcutaneous deposition. Bar chart shows the measured bioluminescence intensity emitted from these mice. Data are presented as the mean±SEM (n=5 each group). P values were determined using a Mann-Whitney U test. FIG. 7K shows a comparison of anti-inflammatory ability of TCP-25 and PHMB, the antiseptic ingredient of Prontosan. THP1-XBlue™_CD14 reporter cells were stimulated with E. coli LPS, in the presence of PHMB and TCP-25. Data are presented as the mean±SEM (n=6). P values were determined using a one-way ANOVA with Tukey's post test. *P≤0.05; **P≤0.01; ***P≤0.001; ****P≤0.0001; NS, not-significant.

FIG. 7L further shows cytokine analysis of the wound fluid collected on days 2, and 3. Data are presented as the mean±SEM (n=6 wounds for gel, n=6 wounds for TCP-25 gel, n=6 wounds for Mepilex Ag, n=6 wounds for Prontosan). P values were determined using a Kruskal-Wallis test with Dunn's post test. *P≤0.05; **P≤0.01.

FIG. 7M further shows IL-1p analysis of the wound fluid collected on days 2, 3, and 5 from established infection model. Data are presented as the mean±SEM (n=6 wounds for gel, n=6 wounds for TCP-25 gel, n=6 wounds for Prontosan). P values were determined using a Kruskal-Wallis test with Dunn's post test. **P≤0.01, NS, not-significant.

FIG. 8A-C shows that TCP-25 targets inflammation in wounds. FIG. 8A shows NF-κB activation in THP-1-XBlue™-CD14 reporter cells in response to stimulation with wound fluid from infected and TCP-25 gel treated minipigs wounds. Data are presented as the mean±SEM (n=6). P values were determined using a Kruskal-Wallis test followed by Dunn's post test. FIG. 8B shows a demonstration that TCP-25 decreases minipig wound fluid's ability to activate inflammation. THP1-XBlue™-CD14 reporter cells were stimulated by wound fluid from minipig infected wounds from days 1, 2, and 3 in the presence of TCP-25. Data are presented as the mean±SEM (n=4). P values were determined using a one-way ANOVA with Tukey's post test. FIG. 8C shows a demonstration that TCP-25 decreases human wound fluid's ability to activate inflammation. THP-1-XBlue™-CD14 reporter cells were stimulated by chronic wound fluid (CWF) from infected wounds from patients, in the presence of TCP-25. CWF1-5 represent five human patients. Data are presented as the mean±SEM (n=6). P values were determined using a one-way ANOVA with Tukey's post test. *P≤0.05; **P≤0.01; ***P≤0.001; ****P≤0.0001; NS, not-significant.

FIG. 9A-B shows rheological properties of TCP-25 gel. Gel strengths of 2% HEC gel without, or with 0.1 or 1% TCP-25 were analyzed on a Kinexus Pro rheometer. FIG. 9A shows flow points (strain) as a measure of gel strength. Data are presented as the mean with 95% confidence interval (n=3). NS, not-significant. P values were determined using a Kruskal-Wallis test with Dunn's post test. FIG. 9B shows representative elastic modulus (G′) and viscous modulus (G″) plotted against strain (n=3).

FIG. 10A-E shows the in vitro release and in vivo pharmacokinetics of TCP-25 gels. (A) In vitro diffusion of TCP-25 from the gel to buffer. TCP-25 gel #1 was prepared using TAMRA-labeled TCP-25 and loaded into the apical compartment of the transwell inserts. Buffer from the basolateral compartment was collected at various timepoints and cumulative fluorescence was measured to assess the diffusion of TCP-25 from gel to the buffer. Control represents a 0.1% solution of TAMRA-TCP-25. Data are presented as the mean±SEM (n=3). (B) Pharmacokinetics of subcutaneously deposited TCP-25 gel #1 spiked with TCP-25 Cy5 and the effect of LPS. To image in vivo pharmacokinetics of the gel, TCP-25 was spiked with Cy5-labeled TCP-25 and subcutaneously deposited on the back of SKH1 hairless mice. In some mice, LPS was added to the gel before injection. In vivo fluorescence imaging was performed using the IVIS spectrum. Representative images show distribution of TCP-25 Cy5 at 1, 6, and 24 h after deposition of the gel. The lighter the color the higher the signal intensity. Bar chart shows fluorescence measured locally (local) around the gel deposition site and the whole body (body) fluorescence. Data are presented as the mean±SEM (n=3). (C) In vivo tissue uptake of TCP-25 in minipigs. TCP-25 gel #2 or #3 spiked with TCP-25 Cy3 was topically applied on either partial thickness wounds (for 2 h) or on intact skin (for 2 and 24 h). Fluorescence imaging of cryosections was used to detect Cy3-TCP-25 (white, examples of specific staining pinpointed by arrows). Nuclei (grey) were counterstained with DAPI nuclear stain (n=3). (D) Uptake of TCP-25 in minipig ex vivo skin model. TCP-25 gel #4 spiked with Cy3-TCP-25 was topically applied on either intact or wounded ex vivo skin (2 and 24 h). Fluorescence imaging of cryosections was used to detect Cy3-TCP-25 (white, examples of specific staining pinpointed by arrows)). Nuclei (grey) were counterstained with DAPI nuclear stain (n=3). (E) TCP-25 in vivo stability in wound dressings and systemic uptake after topical application of TCP-25 gel on minipig wounds. In a minipig model of partial thickness wounds, TCP-25 gel #2 was applied topically and wound fluid from dressings was collected 24 h after application and analyzed using mass spectrometry. Control wounds were treated with gel without TCP-25. To study systemic uptake of TCP-25 after topical application on wounds, plasma from minipigs was collected and analyzed by mass spectrometry (n=4-5). The LOQ for the assay was 100 nM. P values were determined using a Kruskal-Wallis test with Dunn's post test. *P≤0.05; **P≤0.01.

FIG. 11A-B shows the solubility of TCP-25 comparing pH 7.4 and pH 5. Assessed is the solubility of 0.1% TCP-25 in Tris buffer (10 mM or 25 mM Tris) including 2% or 1.9% glycerol and EDTA (2.5 mM) (A) and the solubility of 0.1% TCP-25 in Acetate buffer (10 mM or 25 mM) including 2% and 1.9% glycerol and 2.5 mM EDTA (B). For comparison, pictures of the respective buffers without TCP-25 are shown.

FIG. 12 shows the efficacy of Tris and Acetate based gels comprising 0.1% TCP-25 and 2.5 mM EDTA against S. aureus biofilm. A) shows the effects on the biofilm of Tris buffer based gel formulations (10 mM and 25 mM Tris) comprising TCP-25 alone or in combination with EDTA B) shows the effects on the biofilm of Acetate buffer based gel formulations (10 mM and 25 mM Acetate) comprising TCP-25 alone or in combination with 2.5 mM EDTA.

FIG. 13 shows the efficacy of 0.1% TCP-25 and EDTA combination in Tris and Acetate based gels against P. aeruginosa biofilm. A) demonstrates the effects on the biofilm by Tris buffer based gel formulations (10 mM and 25 mM Tris) and in combination with 2.5 mM EDTA and TCP-25. B) demonstrates the effects on the biofilm by Acetate buffer based gel formulations (10 mM and 25 mM Acetate) and in combination with 2.5 mM EDTA and TCP-25.

FIG. 14 shows the antibacterial effect of gels comprising a combination of TCP-25 and EDTA in a pig skin ex-vivo model. A) shows the number of bacteria (CFU) on the surface of the burn wounds. B) shows the number of bacteria (CFU) found in the tissue after treatment.

FIG. 15 shows the effects of pH and concentration on TCP-25 oligomerization. Figure A) shows a representative pictures of cuvettes containing 300 μM TCP-25 dissolved in 10 mM Tris pH 7.4 or 10 mM Acetate pH 5, immediately after storage at 4° C. (t0 min) and at the indicated time points after incubation at RT. B) shows absorbance and transmittance values at 405 nm for 10-300 μM TCP-25 dissolved in 10 mM Tris at pH 7.4 or in 10 mM NaOAc at pH 5.8 and 5. In C) was 300 μM TCP-25 dissolved in 10 mM Tris at pH 7.4 or in 10 mM NaOAc at pH 5.8 or 5.0, centrifuged and the pellets and supernatants analyzed on SDS-PAGE. The graph shows the TCP-25 concentration after centrifugation±SD. (D) TEM images illustrating that oligomerization is pH and concentration dependent. TCP-25 was dissolved in pH 7.4 and 5.0 buffers at the indicated concentrations and analysed by TEM. All the experiments were performed 3 times (n=3), * indicates P<0.05. P value was determined using one-way ANOVA with Dunnett's multiple comparison test.

FIG. 16 shows structural analyses of TCP-25 oligomers. Figure A) shows α-helical content±SD and was calculated from CD spectra obtained at 222 nm. A significant increase in α-helical content was observed for 300 μM TCP-25 in 10 mM Tris at pH 7.4. * indicates P<0.05, determined using one-way ANOVA with Dunnett's multiple comparison test (n=3). Figure B) shows separation on 4-16% (w/v) BN-PAGE followed by Western blot analysis shows an increased oligomerization of TCP-25 at higher concentrations. One representative image of 3 independent experiments is shown (n=3). In Figure C) TCP-25 was crosslinked with different concentration of BS³ for 30 min and then analyzed on 10-20% Tris-Tricine gel followed by Coomassie staining. Increased concentration of crosslinker yielded TCP-25 oligomers of higher molecular weights. One representative image of 3 independent experiments is shown (n=3). D) Reverse-phase C18 chromatography of TCP-25, in the absence (black line) or in the presence of 145 μM (dashed black line) or 540 μM (gray line) BS³, shows an alteration in the elution profiles.

FIG. 17 shows thermal and chemical denaturation of TCP-25. TCP-25 (10 and 300 μM) in 10 mM Tris at pH 7.4 or in 10 mM NaOAc at pH 5.0 was denatured by increasing temperature (A) or by addition of increasing amounts of urea (B) or Gdn-HCl (C). The unfolding process was analyzed by recording the emission spectra between 300 and 450 nm upon excitation at 280 nm. Representative emission spectra are shown for 300 μM TCP-25 dissolved at pH 7.4 or 5.0 in with different denaturing method (n=3). Below are reported the denaturation curves. In the case of thermal denaturation, data were obtained by fitting the normalized maximum emission fluorescence as a function of the temperature. For the chemical denaturation, results were obtained using the fluorescence ratio (F₃₃₇/F₃₅₀) as a function of the concentration of the chemical agent. Each data point represents the mean±SEM (n=3). (D) Table showing Tₘ and Cₘ±SEM calculated from the denaturation curves obtained from 3 independent experiments done in duplicate (n=3). →indicates shift in the maximum fluorescence intensity (λₘₐₓ); ↑Iₘₐₓ and ↓Iₘₐₓ indicate increase and decrease in maximum fluorescence intensity, respectively.

FIG. 18 shows reversibility of thermal denaturation of TCP-25 at pH 7.4 and pH 5.0. 10 and 300 μM TCP-25 in 10 mM Tris at pH 7.4 (A) or 10 mM NaOAc (B) by exposing the peptide to 100° C. and bringing the temperature back to 20° C. The re-folding was analyzed by recording the intrinsic fluorescence of the peptide. The spectra were collected at 20 (black line), 100 (dashed line), and 20° C. after denaturation at 100° C. (dotted). Each graph is a representative result of 3 independent experiments (n=3).

FIG. 19 shows size of oligomers and their distribution. (A-B) Representative graphs obtained from DLS analysis of 300 μM TCP-25 in 10 mM Tris pH 7.4 or in 10 mM NaOAc pH 5.0. Sizes of oligomers at pH 7.4 (C) and pH 5.0 (D), and their distribution after storage at RT, 4 or −20° C. up to 24 h (C-D) or after 1 week storage (E), are shown. Oligomers were classified in 4 families: small (0.4-5 nm, black bars), medium (20-150 nm, light gray bars), large (200-950 nm, dark gray bars) and giant (1×10³-5×10³ nm, white bars). For each sample, spectra were recorded three times with 11 sub-runs using the multimodal mode. In the graphs the concentration of the oligomers belonging to different families are reported as an average±SD (n=2).

FIG. 20. Inhibitory and bactericidal effects of TCP-25 of SEQ ID NO:1 in various formulations. (A) representative pictures of tubes containing 1.5% HEC gel formulations made in Tris or Acetate buffer (supplemented with 2 or 1.9% glycerol for isotonicity, respectively) with or without 1% TCP-25 and 2.5 mM EDTA. (B-C) Schematic representation of MIC (B) and MBC (C) values obtained for S. aureus, P. aeruginosa and E. coli after treatment with TCP-25 in Tris or Acetate buffer supplemented with various concentrations of EDTA.

FIG. 21. Antibacterial effect of TCP-25 in various formulations. Bar charts demonstrating the bactericidal effects of 80 μM TCP-25 alone or in combination with 2.5 mM EDTA, in either Tris or Acetate buffer. CFU/ml for P. aeruginosa was determined using VCA assay and a 1 hour treatment time (n=4). Data is presented as means±SEM. A one way ANOVA with multiple comparisons was used to determine p values. **P≤0.01, ***P≤0.001, ****P≤0.0001.

FIG. 22. Aggregation of bacterial cells when treated with TCP-25 and/or EDTA. Heat-maps demonstrating the distribution of aggregated bacterial cells in accordance to area. The distribution is presented as the percentage of the total amount of aggregates. Single cells or aggregates smaller than 20 μm² are not represented here. n=3, with 10 images from each treatment in each replicate.

FIG. 23. Antibacterial effects of TCP-25 against S. aureus in a time-kill assay. A) The graphs show bacterial growth over a time period of 24 hours. Formulations contained 80 μM TCP-25 in either Tris or Acetate buffer with or without 2.5 mM EDTA. Samples were taken at 5, 15, 30 min and 1, 3, 6 and 24 hours. Results are presented as CFU/ml. B) The size of bacterial aggregates in the Live/Dead assay are represented as the heat maps. The relative abundance of aggregates for the respective size class is presented as the percentage of total number of the aggregates. Single cells or aggregates smaller than 20 μm, were excluded. Aggregates are representative from 10 images taken from each sample replicate (n=3).

FIG. 24. Antibacterial effects of TCP-25 against P. aeruginosa in a time-kill assay. A) The graph shows bacterial growth over a time period of 24 hours. Formulations contained 80 μM TCP-25 in either Tris or Acetate buffer with or without 2.5 mM EDTA. Samples were taken at 5, 15, 30 min and 1, 3, 6 and 24 hours. Results are presented as CFU/ml. B) Bacterial aggregates imaged in the Live/Dead assay are represented in heat maps, showing the percentage of aggregates in specific sizes that were found in the samples. Single cells or aggregates smaller than 20 μm, are not represented. Aggregates are representative from 10 images taken from each sample replicate (n=3).

FIG. 25. EDTA enhances TCP-25-mediated reduction of biofilm-associated bacteria. A-B) Bar charts demonstrating the reduction of bacterial amount inside a 48-hour mature biofilm. Biofilm was exposed to a solution (A) or gel formulation (B) containing 0.1% TCP-25 in 10 mM Tris at pH 7.4 or 10 mM Acetate at pH 5 with or without 2.5 mM EDTA (n=3). C) CFU/ml from 48 h mature biofilms were counted after treatment with the formulations in a solution form D) CFU/ml from 48 hours mature biofilms treated with TCP-25 and EDTA formulation in a 1.5% HEC gel in either 25 mM Tris or 25 mM Acetate with 1.9% glycerol (n=3). Data is represented as mean±SEM. One way ANOVA with Tukey post hoc multiple comparison was used to determine the p values. *P≤0.05, **P≤0.01, ***P≤0.001, ****P≤0.0001.

FIG. 26. Effects of TCP-25 formulations in a porcine skin wound infection model. A) Dose-dependent antimicrobial effect of TCP-25 on infected ex vivo pig skin. P. aeruginosa bacterial CFU/ml at the wound surface and in the tissue after treatment with increasing doses (0.1, 0.5 or 1%) of TCP-25 in a Tris-based hydrogel (1.5% HEC and 2% glycerol) B) EDTA at the indicated concentrations was added to 0.1% TCP-25 formulated in Acetate buffer at pH 5.0 (1.5% HEC and 2% glycerol). P. aeruginosa bacterial CFU/ml on the surface and in the tissue after treatment were determined. 0.1% TCP-25 hydrogel at pH 7.4 was used for comparison. C) Dose dependence of TCP-25 in presence of 10 mM EDTA. CFU/ml of P. aeruginosa and S. aureus from surface and tissue samples after 2 hours of treatment were determined. Data is presented as mean±SEM. One way ANOVA with Tukey post hoc multiple comparison was used to determine the p values. *P≤0.05, **P≤0.01, ***P≤0.001, ****P≤0.0001.

FIG. 27. Stability of TCP-25 in Acetate buffer with or without EDTA. The peptide was dissolved at 0.1% in Acetate buffer (pH 5) with or without EDTA and stored at RT, 4 or 37° C. before analysis by reverse-phase C18 chromatography. The data are presented as the percentage of total area that corresponds to the sum of the area of all eluted peaks (100%). The amount of TCP-25 after storage is presented in black bars and the degradation products in white bars. na, not analysed; w, weeks; ms, months.

FIG. 28. Stability of TCP-25 at different pHs. The peptide was dissolved at 0.1% in distilled water then the pH was corrected by adding NaOH or HCl to reach the indicated pH. The samples were then stored at RT, 4, 37 or 70° C. before analysis by reverse-phase C18 chromatography. The data are presented as the percentage of total area that corresponds to the sum of the area of all eluted peaks (100%). The amount of TCP-25 after storage is presented in black bars and the degradation products in white bars. d, days; w, weeks; ms, months.

FIG. 29 shows an overview of stability and antimicrobial activity of TCP-25 at different pH and concentration in the presence and absence of EDTA. At pH 7.4, a concentration of TCP-25 above 0.1% increases stability, probably due to oligomerization. At pH 5, EDTA significantly boosts antimicrobial activity. At pH 5 EDTA increases stability, probably due to formation of oligomers with EDTA.

### DETAILED DESCRIPTION OF THE INVENTION

### Definitions

In this specification, unless otherwise specified, “a” or “an” means “one or more”.

As used herein the term “approximately” when used in relation to a numerical value refers to +/−10%, preferably +/−5%, more preferably to +/−1%.

The term ‘amino acid’, as used herein, includes the twenty standard amino acids and their corresponding stereoisomers in the ‘D’ form (as compared to the natural ‘L’ form), omega-amino acids other naturally-occurring amino acids, unconventional amino acids (e.g., α,α-disubstituted amino acids, N-alkyl amino acids, etc.) and chemically derivatised amino acids (see below).

The term “standard amino acid” refers to any of the twenty genetically-encoded amino acids commonly found in naturally occurring peptides. The standard amino acids are referred to herein both by their IUPAC 1-letter code and 3-letter code. The term “standard amino acid” is used to refer both to free standard amino acids, as well as standard amino acids incorporated into a peptide. For the peptides shown, each encoded amino acid residue, where appropriate, is represented by a single letter designation.

As used herein the term “EDTA” refers to ethylenediaminetetraacetic acid.

The term “Flow point” as used herein refers to the value of the shear stress at the crossover point G′=G″, wherein G′ is the storage modulus and G″ is the loss modulus at 1 Hz frequency and 25° C. For example, the flow point can be determined using a Kinexus Pro rheometer (Malvern Panalytical Ltd., Malvern, UK), equipped with a plate-plate geometry and a gap of 1 mm. A shear strain from 0.001 to 10 strain is applied to determine the linear viscoelastic region (LVR), and flow point (shear stress at G′ and G″ crossover) at 1 Hz frequency and 25 C. The flow point is determined directly by the rheometer. In some instances the flow point is provided as the strain at the G′ and G″ crossover, however if nothing else is indicated the flow point is the shear stress at G′ and G″ crossover, typically in Pa.

As used herein the term “hydrogel” refers to a continuous phase of an aqueous solution and a hydrophilic polymer that is capable of swelling on contact with water. The “hydrogel” comprises nanostructures formed of said polymer and water, and typically contain more than 90% water. Hydrogels are typically transparent or translucent, regardless of their degree of hydration. Hydrogels are generally distinguishable from hydrocolloids, which typically comprise a hydrophobic matrix that contains dispersed hydrophilic particles. Hydrogels typically have a flow point of at least 10 Pa, such as at least 15 Pa, for example in the range of 10 to 80 Pa, such as in the range of 40 to 60 Pa.

As used herein the term “hydrophilic polymer” refers to a polymer that is characterized by being soluble in and compatible with water. Typically, a hydrophilic polymer possesses a polymer backbone composed of carbon and hydrogen, and generally possesses a high percentage of oxygen in either the main polymer backbone or in pendent groups substituted along the polymer backbone.

The term “local administration” as used herein refers to any form of administration of the compositions of the invention directly at the intended region of the body to be treated. Frequently, said local administration will be topical administration directly to the site of the disorder. By way of example, if the disorder is a wound, local administration implies that the composition is applied directly on the wound.

As used herein the term “non-ionic polymer” refers to a polymer which in a protic solvent under at room temperature and 1 atm pressure substantially bears no structural units having cationic or anionic groups needing to be offset by counterions to maintain electrical neutrality. In particular, a “non-ionic polymer” according to the invention may be a hydrophilic polymer which does not comprise monomeric units having ionizable functional groups, such as acidic or basic groups. Such a polymer will be uncharged in aqueous solution.

As used herein the term “polymer capable of forming a hydrogel” refers to a hydrophilic polymer that is capable of swelling on contact with water. Useful polymers will absorb at least 10 times, preferably at least 50 times, such as in the range of 50 to 200 times the amount of water compared to the polymer's weight in an anhydrous state.

The term “sequence identity” as used herein refers to the % of identical amino acids or nucleotides between a candidate sequence and a reference sequence following alignment. Thus, a candidate sequence sharing 80% amino acid identity with a reference sequence requires that, following alignment, 80% of the amino acids in the candidate sequence are identical to the corresponding amino acids in the reference sequence. Identity according to the present invention is determined by aid of computer analysis, such as, without limitations, the Clustal Omega computer alignment program for alignment of polypeptide sequences (Sievers et al. (2011 Oct. 11) Molecular Systems Biology 7:539, PMID: 21988835; Li et al. (2015 Apr. 6) Nucleic Acids Research 43 (W1):W580-4 PMID: 25845596; McWilliam et al., (2013 May 13) Nucleic Acids Research 41 (Web Server issue):W597-600 PMID: 23671338, and the default parameters suggested therein. The Clustal Omega software is available from EMBL-EBI at https://www.ebi.ac.uk/Tools/msa/clustalo/. Using this program with its default settings, the mature (bioactive) part of a query and a reference polypeptide are aligned. The number of fully conserved residues are counted and divided by the length of the reference polypeptide. The MUSCLE or MAFFT algorithms may be used for alignment of nucleotide sequences. Sequence identities may be calculated in a similar way as indicated for amino acid sequences. Sequence identity as provided herein is thus calculated over the entire length of the reference sequence.

The term “topical administration” or “topically administering” as used herein refers to the application of a composition to the external surface of a patient, notably to the skin or mucosa. Desirably, the external surface is the skin and topical administration involves application of the composition to intact skin, to broken skin, to raw skin or to an open skin wound.

The term “treatment” as used herein refers to any type of treatment or prevention of a disorder, including improvement in the disorder of the subject (e.g., in one or more symptoms), delay in the progression of the disorder, delay the onset of symptoms or slowing the progression of symptoms. Treatment may also be ameliorating or curative treatment. As such, the term “treatment” also includes prophylactic treatment of the individual to prevent the onset of symptoms.

The term “denaturation” as used herein refers to the process of partial or total alteration of the native secondary, and/or tertiary, and/or quaternary structures of proteins or nucleic acids resulting in a loss of bioactivity. Denaturation can be induced by several factors, for example by application of external stress, e.g. by heating or radiation and/or by incubation with chemical denaturant(s), such as a strong acid or base, a concentrated inorganic salt, an organic solvent (e.g., alcohol or chloroform). Examples of chemical denaturants include urea or guanidinium chloride (Gnd-HCl). The term “thermal denaturation” refers to denaturation induced by increasing the temperature. Chemical denaturation refers to incubating the peptide with increasing concentrations of a chemical denaturant, such as urea or guanidinium chloride (Gnd-HCl).

The terms “Tm” and “Cm” as used herein refer to the denaturation midpoint of a given peptide. It is defined as the temperature (Tₘ) or the concentration of chemical denaturant (Cₘ) at which both the folded and unfolded states are equally populated at equilibrium. Tₘ and Cₘ may for example be determined as described in Example 7.

### Composition

The present invention relates to a composition comprising a compound comprising a TCP peptide, a non-ionic polymer capable of forming a hydrogel and an aqueous solution. Examples of useful compounds comprising TCP peptides, non-ionic polymers and aqueous solutions are described herein below.

The composition may preferably be in the form of a hydrogel or a viscous solution. The form of the composition depends on the intended use or area of application. Preferably, the composition is a hydrogel. Hydrogels are useful for local administration, and may be directly useful for topical administration. Furthermore, hydrogels are particularly suitable for use in the methods of treatment of the invention due to the high water content. Where the composition is a viscous solution, the composition may be suitable for eye, ear or nose drops or sprays. If the composition is a viscous solution it may for example also be applied to a product or absorbed by a product

In embodiments of the invention, wherein the composition is a hydrogel, said hydrogel preferably has a flow point of at least 15 Pa, more preferably of at least 25 Pa, such as in the range of 40 to 60 Pa. It is advantageous that a hydrogel has a suitable flow point in order to be particularly useful for local administration. Thus, it is frequently preferred that the hydrogel is sufficiently thick to largely remain at the site of administration.

It is preferred that TCP peptides diffuse only very slowly from the compositions of the invention. For example, it is preferred that the diffusion rate of TCP peptides from the compositions of the invention into a neighboring buffer solution is so slow that at the most 20%, for example at the most 10% of the TCP peptide has diffused to the buffer solution within 2 hours. This may in particular be the case in embodiments of the invention where the composition is a hydrogel. The diffusion rate may for example be determined as described in the section “TCP-25 gel diffusion in Example 1 below.

It is also preferred that TCP peptides only diffuse slowly from the compositions of the invention when administered to an individual. Thus, it is preferred that upon topical administration of the compositions, e.g. to a wound, then less than 100 nM TCP peptides can be detected in plasma of said individual. This may in particular be the case in embodiments of the invention where the composition is a hydrogel. Furthermore, this may in particular be the case in embodiments of the invention where the composition comprises in the range of 0.08 to 3 wt %, such as in the range of 0.1 to 2% TCP peptides.

The compositions may be used on its own and may for example be administered locally directly to the site of the disorder to be treated. In particular, the composition may be administered topically. The composition may alternatively be used together with a product.

The composition should preferably be pharmaceutically acceptable, i.e. not toxic, and may thus be provided as a pharmaceutical composition. However, it is contemplated that, where the composition is used in a way in which it does not come into contact with human or animal tissue, such as for disinfecting an object, the composition can also be non-pharmaceutically acceptable.

The composition may be subjected to conventional pharmaceutical operations such as sterilisation and/or may contain conventional adjuvants such as preservatives, stabilisers, wetting agents, emulsifiers, buffers, fillers, etc., e.g., as disclosed elsewhere herein.

It will be appreciated by persons skilled in the art that the composition of the invention may be administered locally. Routes of administration include topical, ocular, nasal, buccal, oral, vaginal and rectal administration. In preferred embodiments the compositions of the invention are for use in methods of treatment by topical administration.

The composition is preferably administered to a patient in a pharmaceutically effective amount. By “pharmaceutically effective amount” is meant an amount that is sufficient to produce the desired effects in relation to the condition for which it is administered, i.e. to provide a desired wound healing, antibacterial effect and/or anti-inflammatory effect.

Typically, the TCP peptide is present in said composition in a concentration of at least 0.01 wt %, more preferably in a concentration of 0.01 to 5 wt %, such as 0.08 to 3 wt %, for example in the range of 0.1 to 2%.

Compositions of the invention may have any desirable pH, e.g. a pH in the range of pH 4 to pH 8, such as in the range of pH 5 to pH 8, for example in the range of 7 to 8. Compositions having a pH of more than 6, such as a pH of more than 7, such as a pH in the range of 6 to 8, for example a pH in the range of 7 to 8 may be particularly stable—even in the absence of EDTA. The composition may be administered by single administration or by multiple administrations. The composition may be administered alone or in combination with other therapeutic agents.

### Composition Comprising EDTA

In one embodiment, the invention provides compositions comprising a compound comprising a TCP peptide, EDTA and preferably also an aqueous buffer. Useful TCP peptides and aqueous buffers are described below.

In some embodiments, it is preferred that compositions comprising TCP peptide and EDTA have a pH of at the most 7, though in other embodiments, compositions comprising TCP peptide and EDTA may have any useful pH, for example a pH of at the most 8 or a 10 pH in the range of 3 to 10, such as in the range of 3 to 8, such as in the range of 3.5 to 8, for example in the range of 5 to 8.

Said composition may also comprise a non-ionic polymer capable of forming a hydrogel. Useful non-ionic polymers are described below. In such embodiments, the compositions will typically be in the form of a hydrogel.

In some embodiments it is preferred that compositions comprising EDTA also contain TCP peptides at a high concentration, i.e. a concentration of at least 0.08 wt %, such as a concentration of at least 0.1 wt %, such as a concentration in the range of 0.08 to 3 wt %. The compositions may comprise any useful amount of EDTA. Preferably, the compositions may comprises EDTA at a concentration of at least 1 mM, such as in the range of 1 to 100 mM, preferably at least 1.5 mM, such as at least 2 mM, for example in the range of 2 to 100 mM, such as in the range of 2 to 50 mM, such as in the range of 2 to 25 mM.

In embodiments of the invention wherein the composition comprises a non-ionic polymer capable of forming a hydrogel, said composition may comprise at least 2 mM, such as at least 10 mM, for example at least 15 mM, such as in the range of 15 to 100 mM, for example in the range of 15 to 50 mM EDTA. Preferably, the composition comprises at least 2 mM, such as in the range of 2 to 100 mM, for example in the range of 2 to 50 mM.

This is advantageous because it has surprisingly been shown that EDTA, having essentially no or very limited antibacterial effect on its own, provides a synergistic effect in significantly improving the antibacterial effect of compositions comprising TCP peptides. Thus, adding EDTA to compositions comprising TCP peptides results in improved anti-microbial activity, in particular in improved antibacterial effects against different types of bacteria and even in improved antibacterial effects against biofilm. For example said bacteria may be gram negative bacteria.

In some embodiments it is preferred that compositions comprising EDTA have a relatively low pH, e.g. a pH below 7, because the synergistic antibacterial effect may be more pronounced at low pH.

In one embodiment it is preferred that the pH of the composition comprising EDTA and TCP peptide is lower than 7, preferably lower than 6, such as 5.5 or lower. Said pH may in preferably also be higher than 3, such at least 3.5. Thus, the pH may be in the range of 3 to 6, such as approx. 5. A desired pH may be obtained by using a suitable aqueous buffer, e.g. an Acetate buffer, having the desired pH, as discussed below.

In some embodiments it is preferred that compositions comprising EDTA have a high concentration of TCP-25 peptides, e.g. a concentration of at least 0.08 wt %, because the synergistic antibacterial effect may be more pronounced in such compositions.

As noted above, addition of EDTA to compositions comprising TCP peptides results in synergistically improved anti-microbial activity. However, addition of EDTA may also have other beneficial effects. For example, addition of EDTA may significantly improve the stability of TCP peptides, particularly at low pH.

Thus, in some embodiments of the invention it is preferred that the compositions comprise compounds comprising a TCP peptide as described below as well as EDTA at a concentration of at least 1 mM, such as in the range of 1 to 100 mM, preferably at least 1.5 mM, such as at least 2 mM, for example in the range of 2 to 100 mM, such as in the range of 2 to 50 mM, such as in the range of 2 to 25 mM. Said formulation may have any useful pH, such as a pH of at the most 8, for example a pH of at the most 7, such as a pH lower than 6, such as a pH lower than 5.5. Said pH may in preferably also be higher than 3, such at least 3.5. Such compositions are particularly stable.

Thus, in one embodiment the compositions of the invention still comprise at least 90%, such as at least 95% of the original content of TCP peptide compounds after storage for 2 months at 37° C.

Thus, in one embodiment the compositions of the invention still comprise at least 75%, such as at least 80%, for example at least 90%, such as at least 95% of the original content of TCP peptide compounds after storage for 4 months at 37° C.

Thus, in one embodiment the compositions of the invention still comprise at least 70%, such as at least 80%, for example at least 90%, such as at least 95% of the original content of TCP peptide compounds after storage for 6 months at 37° C.

Thus, in one embodiment the compositions of the invention still comprise at least 90%, such as at least 95% of the original content of TCP peptide compounds after storage for 8 months at room temperature.

### Composition Comprising High Concentration of TCP Peptide

In one embodiment, the invention provides compositions comprising a high concentration of a compound comprising a TCP peptide. Useful compounds and useful TCP peptides are described below.

Said high concentration of a compound comprising a TCP peptide may in particular be that said composition comprises said compound or said TCP peptide at a concentration of at least 0.08 wt %, for example in a concentration of at least 0.1 wt %. Thus, the TCP peptide may be present in said composition in a concentration of in the range of 0.08 to 3 wt %, for example in the range of 0.1 to 2%.

The concentration of the TCP peptide may also be provided as a molar concentration. Translation of a wt % concentration to a molar concentration depends on the specific composition and the specific TCP peptide. A concentration of 0.1 wt % TCP-25 of SEQ ID NO:1 corresponds to a concentration of 300 μM in most aqueous solutions. Thus, said high concentration of a compound comprising a TCP peptide may in particular be that said composition comprises said compound or said TCP peptide at a concentration of at least 0.2 mM, such as at least 0.25 mM, such as at least 0.3 mM. Thus, the compositions of the invention may comprise in the range of 0.2 mM to 100 mM, such as in the range of 0.25 mM to 100 mM, such as in the range of 0.3 mM to 100 mM of a compound comprising a TCP peptide.

Interestingly, the present invention shows that at high concentration TCP peptides may be more stable. Without being bound by theory, it is believed that TCP peptides oligomerises at high concentration, e.g. at TCP peptide concentrations of at least 0.08 wt %, such as at least 0.1 wt %, for example at least 0.2 mM, such as at least 0.25 mM, such as at least 0.3 mM, which may result in higher stability. Said TCP peptide oligomers may for example have a hydrodynamic diameter in the range of 0.2 nm to 10000 nm, such as in the range of 0.4 nm to 8000 nm, such as in the range of 5 nm to 6000 nm, such as in the range of nm to 5000 nm, such as in the range of 0.4 nm to 2000 nm. Said TCP peptide may oligomers may have an increase in α-helical structure.

The oligomerization of the TCP peptide may increases the antibacterial activity and/or the anti-inflammatory activity of the composition.

Interestingly, the TCP peptides or compounds comprising the TCP peptides may be more stable at higher concentrations. In particular, they may be more stable against denaturation, e.g. they may be more stable against exposure to high temperatures, such as to exposure to temperatures in the range of 20-100° C., for example in the range of 30 to 50° C. and/or to incubation at high concentration of denaturant agents.

The stability of TCP peptides may be determined by any useful method, for example TCP peptide stability may be measured by determining the Tₘ of TCP peptides by measuring intrinsic tryptophan fluorescence. This may for example be done as described in Example 7 herein below. Alternatively, the stability of TCP peptides may be determined by determining the Cₘ of TCP peptides in respect of one or more chemical denaturants by measuring intrinsic tryptophan fluorescence. Said chemical denaturants may for example be urea and/or guanidinium chloride (Gnd-HCl). This may for example be done as described in Example 7 herein below.

A high Tₘ and/or a high Cₘ is indicative of high stability. Thus, in some embodiments the compositions of the invention have a Tₘ in respect of the TCP peptides of at least 30° C., preferably of at least 35° C., even more preferably of at least 40° C., wherein said Tₘ preferably is determined as described in Example 7 below. In some embodiments the compositions of the invention have a C_{m urea} in respect of the TCP peptides of at least 0.8 M, preferably of at least 1.0 M, even more preferably of at least 1.1 M, wherein said C_{m urea} preferably is determined as described in Example 7 below. In some embodiments the compositions of the invention have a C_{m Gnd-HCl} in respect of the TCP peptides of at least 0.8 M, preferably of at least 0.9 M, wherein said C_{m Gnd-HCl} preferably is determined as described in Example 7 below.

In one embodiment, the compositions of the invention still comprise at least 90%, such as at least 95% of the original content of TCP peptide compounds after storage for 2 months at 37° C.

In one embodiment the compositions of the invention still comprise at least 75%, such as at least 80%, for example at least 90% of the original content of TCP peptide compounds after storage for 4 months at 37° C.

Thus, in one embodiment the compositions of the invention still comprise at least 70%, such as at least 80%, for example at least 85% of the original content of TCP peptide compounds after storage for 6 months at 37° C.

Thus, in one embodiment the compositions of the invention still comprise at least 90%, such as at least 95% of the original content of TCP peptide compounds after storage for 8 months at room temperature.

Compositions comprising a high concentration of compounds comprising TCP peptides may also comprise EDTA and preferably also an aqueous buffer, for example as described herein above in the section “Composition comprising EDTA”.

Compositions comprising a high concentration of compounds comprising TCP peptides may have any suitable pH, e.g. a pH in the range of pH 4 to pH 8, such as in the range of pH 5 to pH 8, for example in the range of 7 to 8. Compositions comprising a high concentration of compounds comprising TCP peptides and having a pH of more than 6, such as a pH of more than 7, such as a pH in the range of 6 to 8, for example a pH in the range of 7 to 8 may be particularly stable—even in the absence of EDTA.

Compositions comprising a high concentration of compounds comprising TCP peptides may also comprise a non-ionic polymer capable of forming a hydrogel. Useful non-ionic polymers are described below. In such embodiments, the compositions will typically be in the form of a hydrogel.

### Non-Ionic Polymer Capable of Forming a Hydrogel

The invention provides compositions comprising a non-ionic polymer capable of forming a hydrogel.

In the context of the present invention a non-ionic polymer is understood to encompass a polymer which in a protic solvent under at room temperature and 1 atm pressure substantially bears no structural units having cationic or anionic groups needing to be offset by counterions to maintain electrical neutrality. Cationic groups include for example quaternized ammonium groups and protonated amines. Anionic groups include for example carboxyl and sulfonic acid groups.

The non-ionic polymer, also termed non-ionic hydrogel polymer, is a polymer capable of forming a hydrogel when mixed with an aqueous solution or aqueous buffer. Depending on the concentration of the polymer the composition may be in the form of a viscous liquid or a hydrogel, i.e. gel.

The non-ionic polymer should be hydrophilic, and accordingly, the non-ionic polymer is preferably hydroxylated.

Examples of suitable nonionic polymers for use in the present method are polyallylalcohol, polyvinylalcohol, polyacrylamide, polyethylene glycol (PEG), polyvinyl pyrrolidone, starches, such as corn starch and hydroxypropylstarch, alkylcelluloses, such as C₁-C₆-alkylcelluloses, including methylcellulose, ethylcellulose and n-propylcellulose; substituted alkylcelluloses, including hydroxy-alkylcelluloses, preferably hydroxy-C₁-C₆-alkylcelluloses and hydroxy-C₁-C₆-alkyl-C₁-C₆-alkylcelluloses, such as hydroxyethylcellulose, hydroxypropylcellulose, hydroxybutylcellulose, hydroxypropylmethylcellulose, and ethylhydroxyethylcellulose. Mixtures of the aforementioned may also be employed.

In one embodiment the non-ionic polymer is selected from the group consisting of hydroxyethyl cellulose (HEC), hydroxypropyl cellulose (HPC), Hydroxypropyl Methylcellulose (HPMC), poly(vinyl)alcohol (PVA), polyacrylamide (PA), polyethylene glycol (PEG) and polyvinyl pyrrolidone, and mixtures thereof,

In one embodiment the non-ionic polymer is selected from the group consisting of hydroxyalkyl celluloses, preferably hydroxy-C₁-C₆-alkylcelluloses or hydroxy-C₁-C₆-alkyl-C₁-C₆-alkylcelluloses.

Among the non-ionic polymers, hydroxyethyl cellulose (HEC) and hydroxypropyl cellulose (HPC) are preferred.

In preferred embodiments, the concentration of the non-ionic polymer in the compositions of the invention is sufficient to obtain a composition with a flow point of at least 10 Pa, such as at least 15 Pa, for example in the range of 10 to 80 Pa, such as in the range of 40 to 60 Pa.

The flow point may for example be measured as detailed in the example section.

The compositions of the invention may in particular comprise the non-ionic polymer in a concentration of at least 0.05 wt %, for example in a concentration of at least 0.09 wt %, such as in a concentration of at least 0.1 wt %, for example in a concentration of at least 0.5 wt %, such as in a concentration of at least 0.8 wt %, for example in a concentration of at least 1.0 wt %, for example in the range of 0.09 to 4 wt %, such as in the range of 0.1 to 3%, more preferably in the range of 1.0 to 2.5 wt %. This may for example be the case, when the non-ionic polymer is HEC.

In one embodiment the concentration of non-ionic polymer is sufficient to obtain a viscosity of the composition of at least 10 mPas, and preferably no more than 100000, such as 14000 mPas. At 10 mPas the composition is typically in the form of a viscous solution, whereas at 100000 mPAs the composition may be in the form of a dense gel.

### Aqueous Solution

The compositions of the present invention may comprise an aqueous solution. In particular, said aqueous solution may be an aqueous buffer.

An aqueous solution or an aqueous buffer contains water. When the aqueous solution is an aqueous buffer it also comprise components of a buffer system, i.e. weak bases or acids and their conjugate acids and bases, for obtaining an aqueous buffer capable of providing a generally stable pH. Examples of buffers are Trizma, Bicine, Tricine, MOPS, MOPSO, MOBS, Tris, Hepes, HEPBS, MES, phosphate, carbonate, Acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

In one embodiment it is preferred that the pH of the composition is lower than 7, preferably lower than 6, more preferably 5.5 or lower, and higher than 3, such at least 3.5. Thus, the pH may be in the range of 3 to 6, such as approx. 5. This is advantageous because it has surprisingly been shown that a lower pH significantly increases the antibacterial effect of the hydrogel composition, in particular in the presence of EDTA. Furthermore, a pH lower than 7 also increases the solubility of TCP peptides. Solubility may for example be determined by visual inspection as described in Example 4. A desired pH may be obtained by using a suitable aqueous buffer, having the desired pH, as discussed above.

In such embodiments the aqueous solution or aqueous buffer may be an Acetate buffer comprising Acetate, preferably at a concentration of 5 to 50 mM, more preferably at a concentration of in the range of 10 to 30 mM. Said Acetate buffer may have a pH in the range of 3 to 6, such in the range of 3.6 to 5.8, for example approx. 5.

An Acetate buffer, such as a sodium Acetate buffer, is particularly suitable as buffer for obtaining a pH of 3.6 to 5.8. Typically, for a 10 mM sodium Acetate buffer, a pH of 5 is obtained, see example 2. However, as explained above, other aqueous buffers can be used to obtain the desired pH.

In an alternative embodiment the pH of the composition is between 7 and 8, preferably 7.4.

A neutral pH may in some cases preferred as this provides the composition with a pH close to that of the human or animal body, and hence decreases the risk of irritation from the composition when administered to a human or animal.

In such embodiments the aqueous solution or aqueous buffer may be a Trisaminomethane (Tris) buffer comprising Trisaminomethane, preferably at a concentration of 5 to 50 mM, such as in the range of 10 to 30 mM.

A Tris buffer may for example be used to obtain a pH of approx. 7.4, However, as explained above, other aqueous buffers can be used to obtain the desired pH.

The aqueous solution or aqueous buffer may additionally or alternatively comprise further components such as diluents, adjuvants, tonicity regulators and/or excipients. Generally such further components should be pharmaceutically acceptable.

The term “diluent” is intended to mean an aqueous or non-aqueous solution with the purpose of diluting the peptide in the composition. The diluent may be one or more of saline, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the peptide. The adjuvant may be one or more of colloidal silver, or zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and Acetates of different acyl composition. The adjuvant may also be a compound with antibacterial and/or antiinflammatory properties.

In one embodiment it is preferred that the compositions of the invention do not comprise any ionic polymers. In particular, it may be preferred that said compositions do not comprise any cationic polymers.

The “excipient” may be any useful excipient, such as one or more of polymers, lipids and minerals.

The term “tonicity regulator” refers to a compound capable of regulating the tonicity of the composition. In general, it is preferred that the compositions of the invention are either isotonic or somewhat hypotonic. A hypotonic composition may for example have a tonicity, which is 50 to 99% of isotonic. The tonicity regulator for example be a salt or glycerol.

In one embodiment the composition further comprises glycerol, preferably at a concentration of 1 to 2.5%, preferably at a concentration of 1.2 to 2.2 vol %. The concentration is provided as the concentration in the composition, e.g. the concentration in the hydrogel. In general, a composition comprising 2% glycerol will be isotonic, and thus in some embodiments, the composition may comprise approx. 2% glycerol. However, in other embodiments, the composition is hypotonic, in which case it may comprise in the range of 1.2 to 1.9% glycerol.

### Compound Comprising a TCP Peptide

The invention relates to compositions comprising a compound comprising a TCP peptide. Useful TCP peptides are described herein below in the section TCP peptide. In some embodiments said compound may consist of said TCP peptide, e.g. TCP-25. However, in other embodiments, the compound may comprise a TCP peptide conjugated to one or more additional moieties. For example the TCP peptide may comprise one or more amino acids that are modified or derivatised, for example by PEGylation, amidation, esterification, acylation, acetylation and/or alkylation.

For example, the TCP peptide (e.g. TCP-25) may be modified or derivatised as described in international patent application WO2011/036442 on p. 11, I. 1 to p. 15, I. 14.

The compound may also be a pharmaceutically acceptable acid or base addition salt of the TCP peptide. The acids which are used to prepare the pharmaceutically acceptable acid addition salts of the TCP peptides are those which form non-toxic acid addition salts, i.e. salts containing pharmacologically acceptable anions, such as the hydrochloride, hydrobromide, hydroiodide, nitrate, sulphate, bisulphate, acid, Acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

### TCP Peptides

The invention relates to compositions comprising thrombin-derived C-terminal (TCP) peptides. As used herein the term “TCP peptide” refers to a peptide comprising or consisting of the amino acid sequence
X₁-X₂-X₃-X₄-X₅-X₆-W-X₈-X₉-X₁₀, wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100, for example from 20 to 100,
such as from 18 to 35, for example from 18 to 25 amino acid residues.

In one embodiment, the TCP peptide comprises or consists of the amino acid sequence

X₁-X₂-X₃-X₄-X₅-X₆-W-X₈-X₉-X₁₀-X₁₁-X₁₂-X₁₃, wherein
X_{4, 6, 9, 11} is any standard amino acid,

### X₁, is I, L or V

X₂ is any standard amino acid except C

### X₃ is A, E, Q, R or Y

X₅ is any standard amino acid except R

### X₈ is I or L

X₁₀ is any standard amino acid except H

### X₁₂ is I, M or T

### X₁₃ is D, K, Q or R

and wherein said peptide has a length of from 20 to 100, such as from 18 to 35, for example from 18 to 25 amino acid residues.

In one embodiment, the TCP peptide comprises or consists of the amino acid sequence

X₁-X₂-X₃-X₄-X₅-X₆-W-X₈-X₉-X₁₀-X₁₁-X₁₂-X₁₃-X₁₄-X₁₅-X₁₆-X₁₇, wherein
X_{4, 6, 9, 11, 14, 15} is any standard amino acid

### X₁ is I, L or V

X₂ is any standard amino acid except C

### X₃ is A, E, Q, R or Y

X₅ is any standard amino acid except R

### X₈ is I or L

X₁₀ is any standard amino acid except H

### X₁₂ is I, M or T

### X₁₃ is D, K, Q or R

### X₁₆ is G or D

### X₁₇ is E, L, G, R or K

and wherein said peptide has a length of from 20 to 100, such as from 18 to 35, for example from 18 to 25 amino acid residues.

In one embodiment, the TCP peptide has a length of 18 to 35 amino acids, preferably 18-25 amino acids, and comprises or consists of any of the amino acid sequences

(SEQ ID NO 1)
GKYGFYTHVFRLKKWIQKVIDQFGE
(SEQ ID NO 2)
FYTHVFRLKKWIQKVIDQFGE,
(SEQ ID NO 3)
GKYGFYTHVFRLKKWIQKVI,
(SEQ ID NO 4)
HVFRLKKWIQKVIDQFGE,
(SEQ ID NO: 5)
KYGFYTHVFRLKKWIQKVIDQFGE
(SEQ ID NO: 6)
GKYGFYTHVFRLKKWIQKVIDQF
(SEQ ID NO: 7)
GKYGFYTHVFRLKKWIQKV.

In one embodiment the TCP peptide has a length of 18 to 35 amino acids, preferably 18-25 amino acids, and comprises or consists of any of the amino acid sequences

(SEQ ID NO 1)
GKYGFYTHVFRLKKWIQKVIDQFGE
(SEQ ID NO 2)
FYTHVFRLKKWIQKVIDQFGE,
(SEQ ID NO 3)
GKYGFYTHVFRLKKWIQKVI,
or
(SEQ ID NO 4)
HVFRLKKWIQKVIDQFGE.

In one preferred embodiment the TCP peptide has a length of 18 to 35 amino acids, preferably 18-25 amino acids, and comprises or consists of any of the amino acid sequences

(SEQ ID NO: 1)
GKYGFYTHVFRLKKWIQKVIDQFGE,
or
(SEQ ID NO: 3)
GKYGFYTHVFRLKKWIQKVI.

It is preferred that the TCP peptide is capable of simultaneously binding both to lipopolysaccharides and to the LPS-binding hydrophobic pocket of CD14.

The example section shows the antibacterial effect of several TCP peptides including TCP-25. The term “TCP-25” as used herein refers to a peptide consisting of the amino acid sequence GKYGFYTHVFRLKKWIQKVIDQFGE (SEQ ID NO 1). The example section further shows that TCP-25 can be cleaved into the multiple peptides including FYT21, GKY20 and HVF18, i.e. SEQ ID NO 2, 3 and 4, and that these peptides, which are similar to TCP-25, are also antibacterial. Said peptides also include the amino acid sequences necessary for both lipopolysaccharide (LPS) binding and CD14 binding. Accordingly, the TCP peptide may in some embodiments comprise or consists of any of these peptides (TCP-25FYT21, GKY20 and HVF18). Preferably the peptide has a length of 18-25 amino acids but, as long as the peptide is based on any of these peptides, the peptide may be up to 35 amino acids long.

In an alternative preferred embodiment the peptide has at least 90% sequence identity with the amino acid sequence GKYGFYTHVFRLKKWIQKVIDQFGE (SEQ ID NO. 1), and preferably the TCP-25 peptide has the amino acid sequence

(SEQ ID NO. 1)
GKYGFYTHVFRLKKWIQKVIDQFGE

As different amino acids may have side chains providing similar properties, and as the biological effect of a peptide is caused by the side chains of several amino acids cooperating to provide a specific steric structure or chemical/electrical local environment, e.g. by hydrophobic side chains, the peptide may alternatively have at least 90% sequence identity to the TCP-25 sequence. Thus the peptide may correspond to the TCP-peptide wherein one or more, up to 1/10^{th}, i.e. up to 3 of the amino acids in the TCP-25 peptide, have been replaced by other amino acids. Despite such replacement, the general activity of the peptide will resemble that of the TCP-25 peptide, (SEQ ID NO 1).

It is preferred that the TCP peptide is capable of simultaneously binding both to lipopolysaccharides and to the LPS-binding hydrophobic pocket of CD14.

### Method of Treatment

The compositions of the invention may be for use in a method of treatment. In particular the compositions may be for use in a method of local treatment of a disorder. Said disorder may be any disorder for which local treatment is adequate. Accordingly, the methods may involve local administration of the compositions of the invention directly to the local site affected by the disorder.

For example, the disorder may be disorder of the skin, ears, eyes or nose. Thus, the method of treatment may involve local administration to affected areas of the skin, ears, eyes or nose.

In one embodiment the method of treatment involves topical administration, for example topical administration to the skin.

In a preferred embodiment, the compositions of the invention are for use in a method of treatment of a skin disorder. In particular, the compositions may be for a method of treating wounds.

The composition or product of the invention may be applied directly to the skin or wound. As shown in the examples the compositions of the invention are antibacterial and reduces inflammation and provides a faster and better wound healing compared to prior art techniques and products.

In preferred embodiments the method of wound treatment comprises a method of treating burn wounds and non-healing ulcers. In an alternative embodiment the method of wound treatment comprises a method of treating surgical wounds.

These types of wound may require special measures, and the compositions of the invention are particularly useful for treatment of such wounds, because they provide both an antibacterial effect and an anti-inflammatory effect.

The composition or product may for example be applied directly to the wound, or be applied in the form of any of the products described herein, e.g. as a bandage, or suture, etc for treating the surgical wound.

The disorder to be treated may in particular be a disorder comprising an inflammation or a disorder associated with an inflammation or a disorder at risk of contracting an inflammation. In particular, the disorder may be a disorder comprising or associated with a local inflammation.
The disorder to be treated may in particular be a disorder comprising an infection or a disorder associated with an infection or a disorder at risk of contracting an infection. In particular, the disorder may be a disorder comprising or associated with a local infection. Said infection may in particular be an infection by bacteria, i.e. a bacterial infection.
Said bacteria may be any infectious bacteria. For example, the bacteria may be Gram, negative or Gram positive bacteria. Thus, the bacteria may be of a genus selected from the group consisting of Staphylococcus, Enterococcus, Streptococcus, Corynebacterium, Escherichia, Klebsiella, Stenotrophomonas, Shigella, Moraxella, Acinetobacter, Haemophilus, Pseudomonas and Citrobacter. In one embodiment, the bacteria are selected from the group consisting of S. aureus and P. aeruginosa. In another embodiment, the bacteria are gram negative bacteria.

Said bacteria may even be multiresistant bacteria. Surprisingly, the compositions of the invention (and accordingly the products) are capable of providing an antibacterial effect against several multiresistant bacteria, i.e. bacteria which are resistant to several known antibiotics. The composition thus provides an additional way of treating these bacteria, including treating wounds infected by these bacteria.

The individual in need of treatment may be any individual. Typically, said individual is a mammal, and preferably said individual is a human being. In one embodiment the individual is an individual suffering from diabetes, arterial insufficiency or venous insufficiency. Individuals suffering from diabetes, arterial insufficiency or venous insufficiency frequently also suffers from non-healing ulcers, and the disorder may thus be a non-healing ulcer of an individual suffering from diabetes, arterial insufficiency or venous insufficiency.

In one embodiment, the composition or product is for use in a method of treatment of a disorder of the skin, ears, eyes or nose, e.g. for treatment of a wound. Said disorder may for example be selected from the group consisting of atopic dermatitis, impetigo, chronic skin ulcers, infected acute wound and burn wounds, acne, external otitis, fungal infections, pneumonia, seborrhoic dermatitis, candidal intertrigo, candidal vaginitis, oropharyngeal candidiasis, eye infections and nasal infections. Furthermore, the disorder may be burn wounds, surgical wounds or skin trauma.

Since aforementioned disorder are often accompanied with complications such as bacterial infection and/or inflammation, the anti-infectious and anti-inflammatory treatment provided by the compositions of the invention is beneficial.

The treatment may be ameliorating treatment, curative treatment and/or preventive treatment. Thus, the compositions of the invention may be employed in methods for reducing the risk of infection and/or inflammation associated with a disorder. For example, the compositions may be administered to a wound in order to reduce the risk of infection and/or inflammation in said wound. The compositions of the invention may however also be administered to individuals already suffering from a local infection and/inflammation.

### Product

The invention also provides products comprising the compositions according to the invention. The product may for example be a product, which can aid local administration of the compositions of the invention.

In such embodiments the product may for example be selected from the group consisting of gels, drops, sprays, creams, liquids, wound irrigation liquids, contact lens liquids, ointments, suture, prosthesis, implant, wound dressing, plaster, catheter, skin graft, skin substitute, and bandage.

Drops and sprays may for example be configured, (i.e. formulated) for applying the composition to ears, eyes, or the nose. The composition may for example be formulated as a viscous liquid easily applicable to eyes or ears, and may alternatively be formulated as hydrogel for easy application to ears.

Products, e.g. hydrogels, drops, sprays, wound dressings, plasters, skin substitutes and bandages may be configured or formulated for administration of the composition to the skin or to other epithelial surfaces or to a wound.

In general, the composition and the product may be formulated for local administration, and in particular for topical administration.

In such embodiments, the composition may be coated, painted, or sprayed onto the product, or the composition may be adsorbed or absorbed by the product.

In so doing, the composition may impart antibacterial and anti-inflammatory properties to the product.

The term ‘coated’ as used herein refers to the composition being applied to the surface of the product. Thus, the product may be painted or sprayed with a solution comprising the composition. Alternatively, the product may be dipped in a reservoir of the composition.

Advantageously, the product is impregnated with the composition. By ‘impregnated’ is meant that the composition is absorbed or adsorbed with the product.

### Items

The invention may further be defined by any one of the following items:

1. A composition comprising:
c) a compound comprising a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues,
d) a non-ionic polymer capable of forming a hydrogel when mixed with an aqueous solution, and
e) an aqueous solution.
   2. A composition comprising:
a) a compound comprising a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues,
b) a non-ionic polymer capable of forming a hydrogel when mixed with an aqueous solution, and
c) an aqueous solution
   wherein
      i. the concentration of the compound in the composition is at least 0.08 wt % and/or
      ii. the non-ionic polymer is present in said composition at a concentration of at least 0.05 wt %.
         3. The composition according to any one of the preceding items, wherein the composition is a hydrogel or a viscous solution, preferably the composition is a hydrogel.
         4. The composition according to any one of the preceding items, wherein the non-ionic polymer is hydroxylated.
         5. The composition according to any one of the preceding items, wherein the non-ionic polymer is selected from the group consisting of polyallylalcohol, polyvinylalcohol, polyacrylamide, polyethylene glycol (PEG), polyvinyl pyrrolidone, starches, such as corn starch and hydroxypropylstarch, alkylcelluloses, such as C₁-C₆-alkylcelluloses, including methylcellulose, ethylcellulose and n-propylcellulose; substituted alkylcelluloses, including hydroxy-alkylcelluloses, preferably hydroxy-C₁-C₆-alkylcelluloses and hydroxy-C₁-C₆-alkyl-C₁-C₆-alkylcelluloses, such as hydroxyethylcellulose, hydroxypropylcellulose, hydroxybutylcellulose, hydroxypropylmethylcellulose, ethylhydroxyethylcellulosen and mixtures of the aforementioned.
         6. The composition according to any one of the preceding items, wherein the non-ionic polymer is selected from the group consisting of hydroxyethyl cellulose (HEC), hydroxypropyl cellulose (HPC), Hydroxypropyl Methylcellulose (HPMC), poly(vinyl)alcohol (PVA), polyacrylamide (PA), polyethylene glycol (PEG) and polyvinyl pyrrolidone, and mixtures thereof,
         7. The composition according to any one of the preceding items, wherein the non-ionic polymer is selected from the group consisting of hydroxyalkyl celluloses, preferably from the group consisting of hydroxyethyl cellulose (HEC) and hydroxypropyl cellulose (HPC).
         8. The composition according to any one of the preceding items, wherein the concentration of the non-ionic polymer in the compositions of the invention is sufficient to obtain a composition with a flow point of at least 10 Pa, such as at least 15 Pa, for example in the range of 10 to 80 Pa, such as in the range of 40 to 60 Pa.
         9. The composition according to any one of the preceding items, wherein the non-ionic polymer is present in said composition at a concentration of at least 0.05 wt %, for example in a concentration of at least 0.09 wt %, such as in a concentration of at least 0.1 wt %, for example in a concentration of at least 0.5 wt %, such as in a concentration of at least 0.8 wt %, for example in a concentration of at least 1.0 wt %, preferably a concentration in the range of 0.09 to 4 wt %, more preferably in the range of 1 to 3 wt %.
         10. The composition according to any one of the preceding items, wherein the non-ionic polymer is present in said composition at a concentration of at least 1 wt %.
         11. The composition according to any of the preceding items, further comprising glycerol, preferably at a concentration of 1 to 3 vol %, more preferably at concentration of 1 to 2 vol %.
         12. The composition according to any one of the preceding items, wherein the aqueous solution is an aqueous buffer.
         13. The composition according to any of the preceding items, further comprising EDTA.
         14. A composition comprising:
d) a compound comprising a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues, and
e) EDTA and
f) an aqueous buffer,
   wherein the composition has a pH of at the most 7.
   15. A composition comprising:
a) a compound comprising a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues, and
b) EDTA and
c) an aqueous buffer,
   wherein
i. the composition has a pH of at the most 8 and/or
ii. the concentration of the compound in the composition is at least 0.08 wt %.
   16. A composition comprising:
a) a compound comprising a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
      wherein said peptide has a length of from 10 to 100 amino acid residues, and
      wherein the concentration of the compound in the composition is at least 0.01 wt %, preferably at least 0.08 wt %, for example in the range of 0.08 to 3 wt %.
      17. A composition comprising:
a) a compound comprising a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
      wherein said peptide has a length of from 10 to 100 amino acid residues, and
      wherein the concentration of the compound in the composition is at least 0.2 mM, such as at least 0.25 mM, such as at least 0.3 mM.
      18. The composition according to any one of the preceding items, wherein the peptide comprises or consists of the amino acid sequence
      X₁-X₂-X₃-X₄-X₅-X₆-W-X₈-X₉-X₁₀-X₁₁-X₁₂-X₁₃, wherein
      X_{4, 6, 9, 11} is any standard amino acid,

### X₁, is I, L or V

X₂ is any standard amino acid except C

### X₃ is A, E, Q, R or Y

X₅ is any standard amino acid except R

### X₈ is I or L

X₁₀ is any standard amino acid except H

### X₁₂ is I, M or T

### X₁₃ is D, K, Q or R

and wherein said peptide has a length of from 20 to 100 amino acid residues.
19. The composition according to any one of the preceding items, wherein the peptide comprises or consists of the amino acid sequence
X₁-X₂-X₃-X₄-X₅-X₆-W-X₈-X₉-X₁₀-X₁₁-X₁₂-X₁₃-X₁₄-X₁₅-X₁₆-X₁₇, wherein
X_{4, 6, 9, 11, 14, 15} is any standard amino acid

### X, is I, L or V

X₂ is any standard amino acid except C

### X₃ is A, E, Q, R or Y

X₅ is any standard amino acid except R

### X₈ is I or L

X₁₀ is any standard amino acid except H

### X₁₂ is I, M or T

### X₁₃ is D, K, Q or R

### X₁₆ is G or D

### X₁₇ is E, L, G, R or K

and wherein said peptide has a length of from 20 to 100 amino acid residues.
20. The composition according to any one of the preceding items, wherein the peptide has a length of 18 to 35 amino acids, preferably 18-25 amino acids, and comprises or consists of any of the amino acid sequences

(SEQ ID NO 1)
GKYGFYTHVFRLKKWIQKVIDQFGE
(SEQ ID NO 2)
FYTHVFRLKKWIQKVIDQFGE,
(SEQ ID NO 3)
GKYGFYTHVFRLKKWIQKVI,
(SEQ ID NO 4)
HVFRLKKWIQKVIDQFGE,
(SEQ ID NO: 5)
KYGFYTHVFRLKKWIQKVIDQFGE
(SEQ ID NO: 6)
GKYGFYTHVFRLKKWIQKVIDQF
(SEQ ID NO: 7)
GKYGFYTHVFRLKKWIQKV.

21. The composition according to any one of the preceding items, wherein the peptide has a length of 18 to 35 amino acids, preferably 18 to 25 amino acids, and comprises or consists of any of the amino acid sequences

(SEQ ID NO: 1)
GKYGFYTHVFRLKKWIQKVIDQFGE,
or
(SEQ ID NO: 3)
GKYGFYTHVFRLKKWIQKVI.

22. The composition according to any one of the preceding items, wherein the peptide has at least 90% sequence identity with the amino acid sequence GKYGFYTHVFRLKKWIQKVIDQFGE (SEQ ID NO. 1), preferably the peptide consists of the amino acid sequence GKYGFYTHVFRLKKWIQKVIDQFGE (SEQ ID NO. 1).
23. The composition according to any one of the preceding items, wherein the peptide is capable of simultaneously binding both to lipopolysaccharides and to the LPS-binding hydrophobic pocket of CD14.
24. The composition according to any one of the preceding items, wherein the peptide is present in said composition in a concentration of at least 0.08 wt %, for example at least 0.1 wt %.
25. The composition according to any one of the preceding items, wherein the peptide is present in said composition in a concentration of at least 0.01 wt %, more preferably in a concentration of 0.01 to 5 wt %, such as 0.08 to 3 wt %.
26. The composition according to any one of items 13 to 25, wherein EDTA is present in said composition in a concentration of at least 1 mM, such as in the range of 1 to 100 mM, preferably at least 1.5 mM, such as at least 2 mM, for example in the range of 2 to 100 mM, such as in the range of 2 to 25 mM.
27. The composition according to any one of items 13 to 25, wherein EDTA is present in said composition in a concentration of at least 2 mM, such as at least 10 mM, for example at least 15 mM, such as in the range of 15 to 100 mM, for example in the range of 15 to 50 mM EDTA.
28. The composition according to any one of the preceding items, wherein the pH of the composition is at the most 7.
29. The composition according to any one of the preceding items, wherein the pH of the composition is lower than 7, preferably lower than 6, more preferably 5.5 or lower.
30. The composition according to any one of the preceding items, wherein the pH of the composition is lower than 7, preferably lower than 6, more preferably 5.5 or lower, and higher than 3, such at least 3.5.
31. The composition according to any one of the preceding items, wherein the pH of the composition is in the range of 3 to 6, such as approx. 5.
32. The composition according to any one of items 12 to 31, wherein the aqueous buffer is an Acetate buffer comprising Acetate, preferably at a concentration of 10-50 mM, more preferably at a concentration of 25 mM, and having a pH of 3.6 to 5, such as 5.
33. The composition according to any one of items 1 to 27, wherein the pH of the composition is at the most 8, such as in the range of 3 to 8, for example in the range of 3.5 to 8, such as in the range of 5 to 8.
34. The composition according to any one of items 1 to 27, wherein the pH of the composition is between 7 and 8, preferably approx. 7.4.
35. The composition according to any one of items 1 to 27 and 33 to 34, wherein the aqueous solution or aqueous buffer is a Trisaminomethane (Tris) buffer comprising Trisaminomethane, preferably at a concentration of 5 to 20 mM, such as approx. 10 mM.
36. The composition according to any one of the preceding items, wherein said peptide within the composition has a Tₘ of at least 30° C., preferably of at least 35° C., even more preferably of at least 40° C.
37. The composition according to any one of the preceding items, wherein the compound comprising said peptide within the composition has a Tₘ of at least 30° C., preferably of at least 35° C., even more preferably of at least 40° C.
38. The composition according to any one of the preceding items, wherein said peptide within the composition has a C_{m urea} of at least 0.8 M, preferably of at least 1.0 M, even more preferably of at least 1.1 M.
39. The composition according to any one of the preceding items, wherein the compound comprising said peptide within the composition has a C_{m urea} of at least 0.8 M, preferably of at least 1.0 M, even more preferably of at least 1.1 M.
40. The composition according to any one of the preceding items, wherein said peptide within the composition has a C_{m Gnd-HCl} of at least 0.8 M, preferably of at least 0.9 M.
41. The composition according to any one of the preceding items, wherein the compound comprising said peptide within the composition has a C_{m Gnd-HCl} of at least 0.8 M, preferably of at least 0.9 M.
42. The composition according to any one of the preceding items, wherein the composition comprises at least 90%, such as at least 95% of the initial content of said compound comprising said peptide after storage for 2 months at 37° C.
43. The composition according to any one of the preceding items, wherein the composition comprises at least 75%, such as at least 80%, for example at least 90% of the initial content of said compound comprising said peptide after storage for 4 months at 37° C.
44. The composition according to any one of the preceding items, wherein the composition comprises at least 70%, such as at least 80%, for example at least 85% of the initial content of said compound comprising said peptide after storage for 6 months at 37° C.
45. The composition according to any one of the preceding items, wherein the composition comprises at least 90%, such as at least 95% of initial content of said compound comprising said peptide after storage for 8 months at room temperature.
46. A product comprising the composition according to any of the preceding items.
47. The product according to item 46, wherein the product is selected from the group consisting of gels, drops, sprays, creams, liquids, wound irrigation liquids, contact lens liquids, ointments, sutures, prostheses, implants, wound dressings, plasters, catheters, skin grafts, skin substitutes, and bandages.
48. The product according to any of the items 46 to 47, wherein the composition is coated, painted, or sprayed onto the product, or wherein the composition is adsorbed or absorbed by the product.
49. A composition according to any of the items 1 to 45, or the product according to any one of items 46 to 48, for use in a method of treatment of a disorder in an individual in need thereof.
50. The composition for use according to item 49, wherein the composition is prepared for local administration.
51. Use of a composition according to any one of items 1 to 45 for the preparation of a medicament for treatment of a disorder in an individual in need thereof.
52. Use according to item 51, wherein the composition is prepared for local administration.
53. A method of treatment of a disorder in an individual in need thereof, wherein the method comprises administration of a therapeutically effective amount of the composition according to any one of items 1 to 45, or the product according to any one of claims 46 to 48 to said individual.
54. The method according to item 53, wherein said administration is local administration.
55. The composition for use, the use or the method according to any one of items 49 to 54, wherein said treatment is selected from the group consisting of ameliorating treatment, curative treatment and preventive treatment.
56. The composition for use, the use or the method according to any one of items 49 to 55, wherein the disorder is a disorder of the skin, ears, eyes or nose.
57. The composition for use, the use or the method according to any one of items 49 to 56, wherein the disorder is a wound.
58. The composition for use, the use or the method according to item 57, wherein the wound is selected from the group consisting of burns and non-healing ulcers.
59. The composition for use, the use or the method according to item 57, wherein the wound is a surgical wound.
60. The composition for use, the use or the method according to any one of items 49 to 59, wherein the disorder comprises an inflammation or is associated with an inflammation.
61. The composition for use, the use or the method according to any one of items 49 to 60, wherein the disorder comprises an infection by bacteria or is associated with infection by bacteria.
62. The composition for use, the use or the method according to item 61, wherein the bacteria is Gram negative or Gram positive.
63. The composition for use, the use or the method according to item 61, wherein the bacteria is Gram negative.
64. The composition for use, the use or the method according to item 61, wherein the bacteria are of a genus selected from the group consisting of Staphylococcus, Enterococcus, Streptococcus, Corynebacterium, Escherichia, Klebsiella, Stenotrophomonas, Shigella, Moraxella, Acinetobacter, Haemophilus, Pseudomonas and Citrobacter.
65. The composition for use, the use or the method according to item 61, wherein the bacteria are selected from the group consisting of S. aureus and P. aeruginosa.
66. The composition for use, the use or the method according to any one of items 61 to 65, wherein the bacteria are multiresistant bacteria.
67. The composition for use, the use or the method according to any one of items 49 to 66, wherein said individual is suffering from diabetes, arterial insufficiency or venous insufficiency.
68. The composition for use, the use or the method according to any one of items 49 to 67, wherein said method of treatment is a topical treatment.

### Example 1

The example describes a hydrogel formulation functionalized with TCP-25. As used in the present examples, the term “TCP-25” refers to a peptide of the following sequence: GKYGFYTHVFRLKKWIQKVIDQFGE (SEQ ID NO:1). The formulation is useful for dual targeting of bacteria and PAMP-induced inflammation. Employing various in vitro assays, different TCP-25 gels were rigorously tested for efficacy against the Gram-positive S. aureus, Gram-negative P. aeruginosa and various other clinical bacterial isolates. The dual antimicrobial and anti-inflammatory action of TCP-25 gels was demonstrated in experimental mouse models of subcutaneous Staphylococcus aureus and Pseudomonas aeruginosa infection and in NF-κB reporter mouse models of endotoxin-induced inflammation. Efficacy of the TCP-25 gels was shown in preclinical porcine partial thickness wound infection models. Pharmacokinetics of TCP-25 in the hydrogel was investigated in vitro, ex vivo and in vivo using fluorescence spectrometry, IVIS bioimaging, and mass spectrometry analyses. To study the fate of active compound in the hydrogel, degradation of TCP-25 was analyzed by mass spectrometry. Bioactivity of major TCP-25 fragments was demonstrated by in vitro assays. Additionally, stability of TCP-25 in gel after long term storage and in plasma was analyzed by mass spectrometry. Finally, efficacy of TCP-25 gel treatment was compared with clinically used wound treatments in a preclinical porcine partial thickness wound model. To further demonstrate the clinical translation, the effect of TCP-25 on the proinflammatory actions of wound fluids from the above porcine infected wounds, as well from patients with non-healing wounds colonized by S. aureus and P. aeruginosa was evaluated using monocyte models.

### Materials and Methods

Ethics statement. All animal experiments are performed according to Swedish Animal Welfare Act SFS 1988:534 and were approved by the Animal Ethics Committee of Malmö/Lund, Sweden (permit numbers M252-11, M131-16, M88-91/14, M5934-19, 8871-19). The use of human wound materials was approved by the Ethics Committee at Lund University (LU 708-01 and LU 509-01).

Statistical analysis. All microbiological and cell culture-based assays show biological replicates and were repeated at least three times. Data are presented as means±SEM. Clinical scoring of wounds is presented as medians. Differences in the mean between two groups were analyzed using Student's t test for normally distributed data and Mann-Whitney test otherwise. To compare means between more than two groups, a one-way ANOVA with post hoc (Tukey) for normally distributed data or Kruskal-Wallis test with post hoc (Dunn's) were used otherwise. Statistical analysis, as indicated in each figure legend, were performed using GraphPad Prism software v8. P values<0.05 were considered to be statistically significant.

Peptides, buffers, and gel formulations. TCP-25 (97% purity, Acetate salt) was synthetized by Ambiopharm (Madrid, Spain). Tetramethylrhodamine (TAMRA), cyanine 3 (Cy3), and cyanine 5 (Cy5)-labeled TCP-25 peptides were synthesized by Biopeptide (San Diego, Calif., USA). The label was added in all cases to the N-terminus of the peptide. The purity (95%) of the labeled peptides was confirmed by mass spectral analysis (MALDI-TOF, Voyager, Applied Biosystems, Framingham, Mass., USA). The gel-forming substances that were used were hydroxypropyl cellulose (HPC, Klucel™ MF, MW 850000; Ashland Industries Europe GmbH, Schaffhausen, Switzerland), hydroxyethyl cellulose (HEC, Natrosol™ 250 HX, MW 1000000; Ashland Industries Europe GmbH, Schaffhausen, Switzerland), and carboxymethyl cellulose (CMC, Blanose™ 7HOF, MW 725000; Ashland Specialties, Alizay, France), and pluronic F-127 (Pluronic© F127, MW 12600; Sigma-Aldrich Chemie GmbH, Steinheim, Germany).

Preparation of TCP-25 hydrogels. For preparation of the gel formulations, HPC, HEC, CMC, or pluronic F-127 were added to 10 mM Tris, pH 7.4 with 1.3% glycerol (for the HEC mixture, the buffer was pre-heated to 56° C.). A magnetic stirrer was used to continuously stir the solution until a homogenous gel was formed. To remove air bubbles, the gel formulation was centrifuged for 3 min (3.5×1000 rpm). The desired amount of TCP-25 peptide was then dissolved in 10 mM Tris (pH 7.4) and 1.3% glycerol buffer, and then added to the gel and the stirring and centrifugation step was repeated. For initial screening of TCP-25 formulations, we used either 40 μM TCP-25 with 1% formulation substance added (HPC, CMC, or pluronic, for FIG. 1A), different TCP-25 concentrations with 0.5% of polymers or pluronic added (for FIG. 1B), or 10 μM TCP-25 with 0.1% polymers or pluronic (for FIGS. 1C and D). Unless expressly stated otherwise, the term “TCP-25 gel #1” refers to a gel comprising 0.1% TCP-25 (0.3 mM), 10 mM Tris HCl at pH 7.4, 1.3% glycerol and 1.5% HEC. TCP-25 gel #1 was used for in vitro and in vivo experiments described in the present examples. For the porcine wound models, the gel contained 0.1% or 1% TCP-25 (0.3 or 3 mM, respectively), 10 mM Tris HCl at pH 7.4, 1.3% glycerol and 2% HEC polymer. In so far as said gel comprises 0.1% TCP-25 it is referred to herein as “TCP-25 gel #2”, whereas said gel comprising 1% TCP-25 is referred to as TCP-25 gel #3. Gel formulations were stored at 4° C. until further use. For fluorescence imaging of TCP-25, the gel was spiked with 2% fluorescently labeled TCP-25 (labeled with TAMRA, Cy3 or Cy5).

Bacterial isolates. The bacterial strains used in this project were E. coli (ATCC 25922), P. aeruginosa (PAO1 and ATCC27853), S. aureus (ATCC 29213), Staphylococcus epidermidis (ATCC 14990), and Enterococcus faecalis (ATCC 29212). The bioluminescent bacteria that were used in this study were P. aeruginosa and S. aureus. We also used clinical isolates of S. aureus (1779, 1781, 2278, 2279, 2404, 2405, 2528, 2788, 2789), P. aeruginosa (10.5, 13.2, 23.1, 27.1, 51.1, 62.2, 15159, 18488), S. epidermidis (2282), and E. faecalis (2374), which were derived either from skin and wound infections. These strains were obtained from the Department of Bacteriology, University Hospital, Lund, Sweden.

Radial diffusion assay (RDA). Bacteria (E. coli, P. aeruginosa, and S. aureus) were grown to mid-logarithmic phase in 10 mL of full-strength (3% w/v) tryptic soy broth (TSB; Becton, Dickinson and Company, Sparks, Md., USA), centrifuged (5600 rpm×10 min), and then resuspended in 10 mM Tris buffer. Then, 4×10⁶ CFU were added to 15 mL of an underlay agarose gel consisting of 0.03% (w/v) TSB, 1% (w/v) low electroendosmosis type (EEO) agarose (Sigma-Aldrich, St. Louis, Mo., USA), and 0.02% (v/v) Tween 20 (Sigma-Aldrich), which were then placed into 144-mm petri dishes. The plates were then prepared by punching 4 mm wells into the agarose gel using a biopsy punch. TCP-25 gel (6 μL) was then added to a well on the agarose and incubated at 37° C. with 5% CO₂ for 3 h to allow diffusion of the peptides into the gel. The underlay gel was covered with 15 mL of molten overlay (6% TSB and 1% low EEO agarose in distilled H₂O), and the plates were then left to incubate at 37° C. for 24 hours. The antibacterial activity of the peptide was visualized as a clear zone around each well and presented as a zone diameter excluding the punch diameter (4 mm). To assess the antibacterial properties of degraded TCP-25, RDA plates were prepared (4×10⁶ CFU E. coli in 15 mL underlay agarose gel as above) and the samples were loaded, as described in the section above. Samples were prepared by mixing the degraded peptide solution with 10 mM Tris at pH 7.4, with or without 0.15 M NaCl.

Viable count assay (VCA). Bacterial strains were grown to mid-logarithmic phase in Todd-Hewitt (TH) media and then centrifuged (5600 rpm×10 min). The bacterial pellet was then washed using 10 mM Tris at pH 7.4, and re-centrifuged for 10 min, after which the pellet was resuspended in the same 10 mM Tris buffer. E. coli, P. aeruginosa, and S. aureus, 1×10⁷ CFU in 50 μL of 10 mM Tris at pH 7.4, were added to tubes containing different TCP-25 gel formulations (0.5% HPC, CMC or pluronic F-127, mixed with either 0, 1, 2, 5 or 10 μM of TCP-25). The tubes were then left to incubate at 37° C. (5% CO₂) for 2 h. Following incubation, serial ten-fold dilutions were performed, and 10 μL×6 from each of the dilutions were plated on TH broth agar plates and left to incubate overnight at 37° C. (5% CO₂), followed by determination of CFU.

Antibacterial effects of TCP-25 gel on bioluminescent bacteria. Bioluminescent P. aeruginosa Xen41 and S. aureus SAP229 were grown to mid-logarithmic phase in TH media, after which they were washed for 20 min in 10 mM Tris at pH 7.4 and 1.3% glycerol buffer (5600 rpm). The bacterial pellet was diluted with 10 mM Tris buffer, and 50 μL from each strain (2×10⁸ CFU/mL) was mixed with 200 μL of gel formulation (1.5% HEC, 10 mM Tris pH 7.4, 1.3% glycerol) with or without 0.1% TCP-25. After incubation for 2 h at 37° C., each sample was gently mixed using a pipette tip. Bioluminescence was measured at 1, 5, 30, and 120 min using an in vivo bioimaging system, IVIS® (Perkin Elmer, USA).

Minimal inhibitory concentration assay. The MIC analysis, which defines the lowest concentration of the antibacterial substance that prevents microbial growth, was performed using a microtiter broth dilution method (Wiegand et al., 2008). In brief, fresh overnight colonies were suspended to a turbidity of 0.5 units and further diluted in Mueller-Hinton broth (Becton Dickinson). To determine the MIC, the TCP-25 was dissolved at a concentration that was ten-times higher than the required range via serial dilutions from a stock solution. Then, 10 μL of each concentration was added to each corresponding well of a 96-well microtiter plate (polypropylene, Costar Corp.). Bacteria were rinsed with Tris (pH 7.4), diluted in MH medium and 90 μL of suspension (approximately 1×10⁵ CFU) was added to each well. The plate was incubated at 37° C. overnight (16-18 h). The MIC was taken as the concentration at which no visible bacterial growth was observed.

NF-κB/AP-1 assay. NF-κB activation was assessed using THP1-Xblue™_CD14 reporter cells (here denoted as THP-1 cells, InvivoGen, San Diego, Calif., USA), according to the manufacturer's instructions. Briefly, THP-1 cells were cultured in RPMI 1640 cell medium, with 10% heat-inactivated FBS, 1% antibiotic-antimycotic (Invitrogen, Carlsbad, Calif., USA), 100 μg/mL G418 (InvivoGen, CA, USA), and 200 μg/mL of Zeocin (InvivoGen, CA, USA). Cells were added into a 96-well plate at 1.8×10⁵ cells/well. Different TCP-25 gel formulations (20 μL) described above (in HPC, CMC or pluronic F-127) were mixed with 20 μL of LPS (1 μg/mL, from E. coli O111::B4, Sigma-Aldrich) and added to the THP-1 cells incubated at 37° C. overnight. Part of the supernatant (20 μL) was mixed with 180 μL QUANTI-Blue reagent (InvivoGen, CA, USA) and further incubated for 1 h (the remainder from the well was used for the MTT assay). The concentrations of secreted embryonic alkaline phosphatase, SEAP (an indicator for NF-κB activation), were quantitatively determined using a spectrophotometer at 600 nm.

MTT assay. The viability of THP-1 cells subjected to the different formulations (with or without TCP-25) was measured using an MTT assay. Sterile filtered MTT (3-(4,5-dimethyl-2-thiazolyl)-2,5-diphenyl-2H-tetrazolium bromide; Sigma-Aldrich Chemie GmbH, Steinheim, Germany) solution (5 mg/mL in PBS) was stored at −20° C. protected from light until it was used. MTT solution (20 μL) was added to each well with the remainder (180 μL) from the NF-κB/AP-1 assay, as described above. Plates were incubated for 90 min in 5% CO₂ at 37° C. After incubation, the plates were centrifuged at 300 g for 10 min and MTT-containing media was removed by aspiration. The blue formazan product was dissolved by adding 100 μL of 100% DMSO (Duchefa Biochemie, Haarlem, The Netherlands) per well. The plates were then gently swirled for 30 min at room temperature to dissolve the precipitate and the absorbance was read at 550 nm. Lysed cells were used as a positive control for the assay. Values for live untreated cells were considered as 100% and values for other treatments are shown in comparison to the untreated live cells.

Wound fluid from patients with non-healing venous ulcers. Wound fluid was collected as described previously (Lundqvist et al., 2004) from patients with chronic venous leg ulcers with an ulcer duration for more than 3 months. Op-Site dressings were applied on the wound and wound fluid was collected via gentle aspiration underneath the films after 2 hours. Sterile wound fluids were obtained from surgical drainages after mastectomy. Wound fluids were centrifuged at 10,000 rpm in an Eppendorf centrifuge, aliquoted, and stored at −20° C. For the present study, wound fluids from patients with positive P. aeruginosa and S. aureus cultures were used.

Circular dichroism spectroscopy. We performed circular dichroism (CD) spectroscopy measurements using a Jasco J-810 spectropolarimeter (Jasco, Easton, Md., USA), equipped with a temperature control unit (25° C.) Jasco CDF-426S Peltier. A sample matrix was prepared using 20 μM TCP-25 diluted and mixed with the different formulation components (TCP-25 with HPC/HEC, CMC, and pluronic F-127, at the ratios of 1:1, and 1:5, respectively), LPS (20 μg/mL), or 10 mM Tris pH 7.4 alone. All mixtures were incubated for 30 minutes at room temperature, after which the samples were placed in a 1-mm quartz cuvette. After extensive purging with nitrogen the samples were scanned over the wavelength interval 200-260 nm (scan speed: 20 nm/min). The average of five scans for each sample were recorded. The baseline (10 mM Tris buffer, formulation component or LPS) was subtracted from the spectra from each sample. We calculated the α-helical content of TCP-25 from molar ellipsometry at 222 nm in the presence of 10 mM Tris buffer, LPS, and the formulation components (at a ratio of 1:5), as described previously.

TCP-25 gel diffusion. A diffusion assay was performed in six-well plates that were equipped with polyethylene terephthalate (PET) inserts (0.4 μm, VWR International, Radnor, Pa., USA). For analysis of peptide release, TCP-25 gel (0.1% TAMRA-TCP-25, 10 mM Tris, pH 7.4, 1.3% glycerol, and 1.5% HEC) was made. Tris glycerol buffer (4 mL) was added to the basolateral compartment of each well. TCP-25 gel (1 mL) was added to the apical compartment. Plates were then incubated at 37° C. A sample of 25 μL was taken from the basolateral compartment at different time points (5 min, 20 min, 30 min, 1 h, 2 h, 6 h, 24 h, and 48 h) and fluorescence was measured using a spectrophotometer at 570 and 583 nm. A 0.1% TCP-25 solution in buffer was used for control.

TCP-25 stability. The stability of TCP-25 in either HEC gel or in buffer (10 mM Tris, pH 7.4, 1.3% glycerol) was investigated using MALDI-TOF mass spectrometry. Samples were prepared (0.1% TCP-25 in 1.5% HEC or in buffer) and then placed in storage for 0, 14, 60, or 180 days. The sample matrix was assigned so that samples from each storage time were also kept at different temperatures −80° C., 4° C., 20° C., and 37° C. After storage, the samples were prepared for mass spectrometry.

Mass spectrometry analysis of TCP-25 fragmentation. TCP-25 (2 μg) in 10 mM Tris was digested with HNE (0.1 μg) in a total volume of 20 μL at 37° C. for 30 min and/or 3 h. Twenty mg of gel formulation (0.1% TCP-25, 10 mM Tris, pH 7.4, 1.3% glycerol, 1.5% HEC) was also digested with 0.2 μg HNE under the same conditions as for the solution. Degradation of TCP-25 in solution and HEC gel was determined using MALDI mass spectrometry and LC-MS/MS analysis.

MALDI mass spectrometry analysis. TCP-25 samples from gel or the solution were diluted in 2% ACN/0.1% TFA and mixed with a solution of 0.5 mg/mL of α-cyano-4-hydroxycinnamic acid (CHCA) in 50% ACN/0.1% TFA solution directly on a stainless MALDI target plate. Typically, 0.5 μL of sample was mixed with 0.5 μL of CHCA solution. Subsequent MS analysis was performed on a MALDI LTQ Orbitrap XL mass spectrometer (ThermoScientific, Bremen, Germany). Full mass spectra were obtained using the FT analyzer (Orbitrap) at 60,000 resolution (at m/z 400). Recording of the mass spectra was performed in the positive mode with an 800-4000 Da mass range. The nitrogen laser was operated at 10.0 μJ with the automatic gain control (AGC) in the off mode and using ten laser shots per position. Evaluation of the spectra was performed using Xcalibur v 2.0.7. software (from Thermo Fisher Scientific, San Jose, Calif., USA).

LC-MS/MS. MS analyses were performed on an Orbitrap Fusion Tribrid MS system (ThermoScientific) that was equipped with a Proxeon Easy-nLC 1000 (Thermo Fisher). A database search was performed with PEAKS 7.5, with the following settings: no enzyme and oxidation of methionine was treated as dynamic modification. The maximum number of post-translational modifications was one per peptide. A MS mass tolerance of 7 ppm and MS/MS mass tolerance of 0.05 Da were used.

Mass spectrometry analysis of TCP-25 in wound fluid and plasma. MS analysis for minipig wound fluid and plasma was performed by Q&Q Labs (Mölndal, Sweden). Briefly, 50 μL of internal standard solution (ISTD) was added to 300 μL of plasma or wound fluid sample and vortexed for 5 s. Then, 1100 μL 0.16% NH₄OH and 30% CAN were added and vortexed for 10 s. Whole samples were then loaded into Oasis WCX columns (Waters, Milford, Mass., USA) and allowed to flow through the column dropwise. After rinsing and elution, samples were dried by evaporation with N₂. Samples were then diluted with 50:50 CAN:MQ, vortexed, and injected onto the LC-MS/MS. The assay range was 30-3000 nM and the limit of quantification (LOQ) was 100 nM.

Stability of TCP-25 in plasma. TCP-25 (10 μM) was incubated in plasma from the different species and in PBS at 37° C. Aliquots were taken at time points 0, 1, 3, and 5 h and prepared for LC-MS analysis by protein precipitation using three volumes of ice-cold acetonitrile with an internal standard to compensate for possible differences in final sample volumes. LC-MS analyses were performed in full scan mode and ratios for peak areas TCP-25 and internal standard were calculated and plotted against time. The obtained k (h⁻¹) values for the disappearance were used to calculate the half-life for TCP-25.

Rheology analysis. Rheology measurements on 2% HEC gel without, or with 0.1 or 1% TCP-25 were performed on a Kinexus Pro rheometer (Malvern Panalytical Ltd., Malvern, UK), equipped with a plate-plate geometry and a gap of 1 mm. A shear strain from 0.001 to 10 was applied to determine the linear viscoelastic region (LVR), and flow point (shear stress at G′ and G″ crossover) at 1 Hz frequency and 25° C. The shear stress (Pa) at the flow point was determined directly by the rheometer. In addition the flow point as strain at G′ and G″ crossover was also determined. The measurements were performed in triplicates.

Mouse inflammation model. Male BALB/c tg(NFk□-RE-Luc)-Xen reporter mice (Taconic Biosciences, Albany, N.Y., USA), 10-12 weeks old, were used to study the anti-inflammatory effects of TCP-25 gel #1 after subcutaneous co-treatment with LPS (E. coli, 5 pg). The dorsum of the mouse was shaved carefully and cleaned. LPS was mixed in 100 μL of TCP-25 gel #1 and immediately injected subcutaneously in anesthetized with isoflurane (Baxter, Deerfield, Ill., USA). Mice were immediately transferred to individually ventilated cages. TCP-25 in the gel formulation was spiked with TCP-25 Cy5 for fluorescence imaging of the peptide. Bioimaging with the IVIS spectrum was used for the longitudinal determination of NF-κB activation. Fifteen minutes before IVIS imaging, mice were intraperitoneally injected with 100 μL of D-luciferin (PerkinElmer, 150 mg/kg body weight). Bioluminescence from the mouse was detected and quantified using Living Image 4.0 Software (PerkinElmer).

Mouse surgical implant model. Male BALB/c mice, 10-12 weeks old, were used for the surgical implant model. The dorsum of the mouse was shaved and cleaned with 70% alcohol. Under isoflurane anesthesia, an approximately 10 mm cut was made on the skin of mouse's back and the tip of the scissors was used to create a small pocket. A 6 mm diameter disc of polyurethane (PU) foam (Mepilex® Transfer, Mölnlycke Heath Care, Gothenburg, Sweden) was inserted under the subcutaneous fascia. Hundred μL of TCP-gel #1 or control gel with LPS (E. coli, 5 μg) was immediately deposited around the PU disc in the subcutaneous pocket and the wound was closed with suture (VICRYL™, Johnson & Johnson, Belgium). Mice were sacrificed at 24 h and PU discs were recovered. Wound fluid was extracted from the PU-discs for further cytokine analysis.

Mouse model of subcutaneous infection. Male SKH-1 hairless mice, 10-12 weeks old, were anesthetized using a mixture of 2% isoflurane and oxygen. TCP-25 gel #1 spiked with TCP-25 Cy5 was used for the fluorescence imaging of the peptide. Overnight cultures of bioluminescent bacteria, P. aeruginosa Xen41 or S. aureus 229, were refreshed and grown to mid-logarithmic phase in TH media. Bacteria were washed for 15 min (5.6 □ 1000 rpm) and diluted with 10 mM Tris buffer (pH 7.4). The formulations were then mixed with 10⁶ CFU of the bacteria. A total of 100 μL of the contaminated mixture (80 μL gel+20 μL bacterial suspension) was injected subcutaneously into the mouse dorsum. In vivo bacterial infection and peptide localization were longitudinally evaluated by measuring bioluminescence (bacteria) and fluorescence (TCP-25 Cy5) in anesthetized mice using IVIS imaging. Animals were imaged either in bioluminescence or in fluorescence mode, and the data obtained were analyzed using Living Image 4.0 Software (PerkinElmer). At termination, tissue samples from the wounds were collected and CFU determined.

For the prevention model, all procedures were similar as above except that the gel (80 μL) was first injected subcutaneously into the dorsum of BALB/c mouse. Thirty minutes later, the bioluminescent S. aureus and P. aeruginosa bacteria (10⁶ CFU in 20 μL) were injected into the site of gel deposition. In vivo bacterial infection was longitudinally evaluated by measuring bacterial bioluminescence using IVIS imaging.

TCP-25 release in mice. Male SKH-1 hairless mice, 10-12 weeks old, were anesthetized using a mixture of 2% isoflurane (Baxter) and oxygen. TCP-25 gel #1 spiked with TCP-25 Cy5 was used for the fluorescence imaging of the peptide. In some mice, TCP-25 gel #1 was mixed with 20 μg of LPS. Gel (100 μL) was injected subcutaneously in isoflurane-anesthetized mice. Release of the peptide was monitored using IVIS imaging in the fluorescence mode and the data obtained were analyzed using Living Image 4.0 Software (PerkinElmer).

Minipig model of partial thickness wounds. To study S. aureus wound infection in vivo, a minipig partial thickness wound model was used. Female Gottingen minipigs weighing 14-16 kg were used. All procedures were performed following strict aseptic techniques by qualified veterinary surgeons. Before wounding, minipigs were acclimatized for 1 week and off-fed the night before wounding. Hair on their back was clipped 24 h before surgery. On the day of wounding, the minipig's back was scrubbed with chlorhexidine (MEDI-SCRUB sponge; Rovers, Oss, Netherlands) and lukewarm water. The dorsum was then shaved, disinfected with chlorhexidine solution (4%), and dried with sterile gauze. Procedures afterwards were performed under general anesthesia. General anesthesia was achieved with a mixture of Tiletamine and Zolacepam (Zoletil 50, Virbac, Sweden). Zoletil induction dose (1 mL/10 kg body weight) was given intramuscularly and anesthesia was maintained intravenously (0.5 mL/10 kg body weight) using a catheter in the auricular vein. During anesthesia, minipigs were supplemented with oxygen via a facemask. Outline for the wounds were marked with a sterile scale ruler and tissue pen. Using an electric dermatome (Zimmer), 12 partial thickness (750 μm deep) wounds measuring 2.5×2.5 cm were created on the minipig's back (six on each side). In initial experiments, the depth of fresh wounds was confirmed by cryosectioning of biopsies and staining with DAPI nuclear stain. A minimum distance of 4 cm was maintained between the wounds. For hemostasis, wounds were covered with sterile gauze. Overnight S. aureus (ATCC 29213) cultures were refreshed and grown to mid-logarithmic phase in TH medium. Bacteria were washed for 15 min (5.6×1000 rpm) and diluted with 10 mM Tris buffer (pH 7.4) to a concentration of 2×10⁸ CFU/mL. For infection, bacteria were suspended in HEC (10⁷ CFU/100 μL) and applied onto the fresh wound surface. Gel (100 μL) was applied onto the uninfected control wounds. Fifteen minutes later, 500 μL of TCP-25 gel #2 or TCP-25 gel #3 or gel only was applied to the wounds and the wounds were then covered with a primary foam dressing (Mepilex® Transfer; Mölnlycke Healthcare, Gothenburg, Sweden). The primary dressing was covered with a transparent breathable fixation dressing (Mepore^{□} Film; Mölnlycke, Gothenburg, Sweden). Dressings were then secured using skin staples (smi, St. Vith, Belgium). For further protection and padding, the wound area was then covered with two layers of sterile cotton gauze and secured with adhesive tape. Finally, a layer of flexible self-adhesive bandage (Vet Flex, Kruuse, Denmark) was used to support and protect the dressings underneath. After recovery from anesthesia, the animals were monitored for any discomfort and provided with water and feed. Animals were housed individually and monitored daily. Twenty-four hours after the wounding and infection, under general anesthesia, the dressings were removed and observations were made. Wounds were documented by imaging and clinical scoring was performed by a qualified veterinarian. Swab samples were taken from the wound surface and used for bacterial analysis. Wound fluid was recovered from the primary dressing was collected each time it was changed and further analyzed. TCP-25 gel #2, TCP-25 gel #3 or gel only (500 μL) was applied to the respective group of wounds and new dressings were applied. In the short-term treatment regimen, dressings were changed every day and minipigs were sacrificed 4 days after infection. In the long-term treatment regimen, dressings were changed on days 2, 3, 5, 7, and 9 and minipigs were sacrificed on day 10. On the day of termination, tissue samples from the wounds were also collected.

For the benchmark comparison study, Prontosan (B Braun, Sempach Switzerland) and Mepilex Ag (Mölnlycke Healthcare, Gothenburg, Sweden) treatment groups were also added. In addition to 1% TCP-25-2% HEC gel, some wounds were treated with Prontosan (500 μL) and others with Mepilex Ag (4.5×4.5 cm). For the wound-healing study in minipigs, partial thickness wounds were created as described above, but no bacterial infection was introduced. Wounds were treated with TCP-25 gel #2, TCP-25 gel #3 or gel only in a long-term treatment regimen.

For the superinfection study, abovementioned short-term treatment regimen was followed with one modification on day 2. Following day 1 wounding, infection with S. aureus and treatment, P. aeruginosa was added to the wounds on day 2. Overnight P. aeruginosa cultures were refreshed and grown to mid-logarithmic phase in TH medium, washed and diluted with 10 mM Tris buffer (pH 7.4). To establish superinfection, P. aeruginosa (10⁵ CFU/100 μL Tris buffer) were applied onto the wound surface. Fifteen minutes later, respective treatments and dressings were applied. Dressings were changed on day 3 and minipigs were sacrificed on day 4.

For the established infection study, abovementioned long-term treatment regimen was followed with one modification on day 1 (FIG. 7E). Immediately after wounding on day 1, S. aureus (10⁷ CFU/100 μL HEC gel) were added to the fresh wound surface but no treatment was applied. Respective treatments were started on day 2 followed by dressings changes on days 3, 5, 7, and 9 and minipigs were sacrificed on day 10.

Identification of contaminating bacteria from minipig wounds with superinfection and preparation of conditioned medium. Fresh swab samples collected from wounds were sent to the Department of Clinical Microbiology, Division of Laboratory Medicine, Sk∪ne University Hospital, Lund and identified using standard microbiological methods. To prepare conditioned medium, bacteria were grown overnight (16 h) in 5 mL TH-media at 37° C. in a shaking incubator (180 rpm) until the optical density reached 1.5. Thereafter, bacteria were centrifuged at 3000×g for 10 min. Supernatant was removed and filter sterilized with a 0.2 μm Filtropur S filter (Sarstedt, Nümbrecht). 1% conditioned media was used for stimulating the THP1-XBlue cells.

In vivo TCP-25 uptake. To study TCP-25 skin penetration and tissue uptake, TCP-25 gel #2 and TCP-25 gel #3 spiked with TCP-25 Cy3 was used. TCP-25 gel #2 or TCP-25 gel #3 was applied either on partial thickness wounds (for 2 h) or on intact skin (for 2 and 24 h) on the minipigs' back. After application of the gel, wounds were dressed as described above. Pigs were sacrificed and biopsies were snap frozen and mounted in OCT compound for cryosectioning. Cryosections were washed in PBS (5 min×1) at room temperature (RT) and 4′,6-diamidino-2-phenylindole (DAPI) solution (0.5 mM in PBS) was used as a nuclear counterstain (1 min, RT). Slides were washed in PBS (5 min x 1, RT), dried, and mounted with antifade mounting medium (PermaFluor, ThermoFisher Scientific). Sections were then imaged using fluorescence microscopy (AxioScope.A1, Carl Zeiss, Germany).

Ex vivo TCP-25 uptake. To study TCP-25 uptake in an ex vivo pig skin model, TCP-25 gel #4 spiked with TCP-25 Cy3 was used. TCP-25 gel #4 comprises 2% TCP-25, 2% HEC, 1.3% glycerol and 10 mM Tris HCl at pH 7.4. Frozen skins were thawed and washed with ethanol (70%) and sterile water. On a petri dish, skins were kept partially submerged in PBS to retain their moisture. TCP-25 gel #4 (50 μL) was applied onto the wounds or intact skin and incubated at 37° C. for a period of 2 and 24 h. At the end of incubation, tissue samples were incised using a surgical scalpel and frozen and mounted in OCT compound for cryosectioning. Cryosections were processed for fluorescence imaging, as described for the in vivo uptake above.

Wound fluid extraction. Wound dressings (Mepilex; Mölnlycke Health Care, Gothenburg, Sweden) from the wound were transferred to a 5 mL prechilled tube and kept on ice. To extract wound fluid, dressings were soaked in 500 μL of cold 10 mM Tris buffer at pH 7.4 and centrifuged for 5 min (2000 g, 4° C.). Extracted wound fluids were aliquoted in prechilled Eppendorf tubes with or without protease inhibitor and stored at −80° C. until further analysis.

Bacterial analysis of wounds. Swab samples collected from wounds were placed in Eppendorf tubes with 500 μL of PBS and vortexed for 30 s. Diluted samples (10-fold dilution in PBS) were plated on TH broth agar and incubated overnight at 37° C. for CFU analysis.

ELISA. Wound fluid collected from dressing material was used to determine IL-6 and TNF-α concentrations. Porcine IL-6 and TNF-α DuoSet^{□} ELISA Kit (R&D Systems, Minneapolis, Minn., USA) were used according to manufacturer's recommendations. For mouse plasma, the IL-6 and TNF-α were assessed using the Mouse Inflammation Kit (Becton Dickinson AB, Franklin Lakes, N.J., USA), according to the manufacturer's instructions.

Single dose toxicity in mice. Ten weeks old female BALB/c mice were given 5 mg TCP-25 (in 100 μL Tris buffer) subcutaneously and sacrificed after 24 h. Tissues (lung, kidney, liver, skin, and spleen) were collected for histological examination and stained with H&E.

Histology. For mouse tissues, harvested skin samples (4 mm or 6 mm by using a biopsy punch) from the infected areas were placed on filter paper to prevent curling and fixed overnight in 4% paraformaldehyde; they were then stored in 70% ethanol. For minipig wounds, tissue samples were harvested using a surgical scalpel and fixed overnight in neutral buffered formalin and then stored in 70% ethanol. After serial dehydration, the tissues were embedded in paraffin blocks, sectioned, and stained with hematoxylin and eosin (H&E). Samples were imaged using bright-field microscopy (Axioplan2, Zeiss, Germany). H&E-stained sections of minipig wound biopsies were examined and scored by an experienced veterinary pathologist (M.P.) in a blinded manner. On a scale of 0-5 (where 0 is worse and 5 is best score), the histological scoring was based on epithelization, granulation tissue, inflammatory cells, abscesses and tissue architecture. For each wound section, five areas were examined under 10× objective which covered 90-100% wound.

### Results

### Evaluation of Peptide Effects and Structure in the Presence of Different Formulation Components

The action of TCP-25 involves structural transitions such as formation of a C-formed turn and a helical structure upon LPS-binding, and relies to some extent on the ability for both bacterial membrane and CD14 interactions. The gel formulation of the present invention supports these TCP-25 functions. The antibacterial activity was determined for TCP-25 alone (see FIG. 1E) or in the presence of hydroxypropyl cellulose (HPC), carboxymethyl cellulose (CMC), or pluronic F-127 (hereafter called pluronic). Radial diffusion analysis (RDA) is an agar diffusion-based method measuring bacteriostatic/bactericidal effects. Using RDA against the Gram-negative Escherichia coli and P. aeruginosa, and the Gram-positive S. aureus, it was possible to demonstrate a largely retained TCP-25 activity against the Gram-negative bacteria E. coli and P. aeruginosa. Addition of CMC, however, inhibited TCP-25 activity against S. aureus peptide activity (FIG. 1A). Analysis using viable count assay (VCA), measuring bactericidal effects in solution, demonstrated that both the anionic CMC polymer and the micelle-forming pluronic interfered with the antibacterial action of TCP-25, while the peptide's antibacterial activity was preserved in HPC (FIG. 1B). Clinical and regulatory considerations also prompted a comparison with the related neutral polymer hydroxyethyl cellulose (HEC), and the results were similar to those obtained with HPC (FIGS. 1E and 1F).

Because the endotoxin-blocking effects of TCP-25 relates to specific interactions with both LPS and cells, it is possible that the structural prerequisites for these anti-inflammatory activities may be separate from those required for the antibacterial action in a specific formulation. The anti-endotoxic activity of TCP-25 in the presence of the different formulation components in vitro using LPS-stimulated THP1-XBlue™-CD14 cells was determined. Cells were incubated with E. coli LPS (10 ng/mL) and with TCP-25 in presence or absence of HPC, CMC, and pluronic. After 18-24 h of incubation, NF-κB and AP-1 activation was assessed. The results showed that CMC in particular, and to a lesser extent pluronic, interfered with TCP-25 anti-endotoxic action. However, HPC did not exert any inhibitory effects on TCP-25 (FIG. 1C). As above, a comparison with the related polymer hydroxyethyl cellulose (HEC) yielded results that were similar to those obtained with HPC (FIG. 1G). Simultaneous analyses of toxic effects of formulation components alone and in combination with TCP-25 were performed and the results showed that the formulation combinations did not affect cell viability, as assessed using an MTT assay (FIG. 1D, FIG. 1H).

Structural features and possible structural transitions of TCP-25 upon LPS binding in the presence of the gel formulation components were analysed using circular dichroism (CD) analysis. Overall, the results showed that, in contrast to the neutral HPC/HEC, the negatively charged CMC induced a strong structural change in TCP-25, which was consistent with an increase in its α-helical content (FIGS. 2A and B). The α-helical content of TCP-25 was calculated from molar ellipsometry at 222 nm in the presence of Tris buffer, LPS, and polymers (at the ratio of 1:5). In presence of LPS, TCP-25 showed a significant (P 50.05) increase in α-helical content in the HEC gel formulation (FIG. 2B), which was similar to its conformational transition induced by LPS in buffer only. Taken together, these structural studies corresponded well with the functional studies, demonstrating that TCP-25 action is facilitated by formulations comprising a neutral polymer, and thus, enabling the peptide's LPS and cell interactions. FIG. 2C depicts the schematic description of TCP-25 peptide and LPS interaction in the presence of the studied gel components.

Based on the above data, a gel base consisting of 1.5% HEC polymer, 1.3% glycerol (for isotonicity), and 10 mM Tris pH 7.4 was used for further studies. Initial dose-response studies using S. aureus and P. aeruginosa showed that doses of 0.01-0.05% TCP-25 (0.1-0.5 mg/ml, corresponding to 0.03-0.15 mM μM TCP-25) in HEC gel exhibited antibacterial effects (FIG. 2C). A dose of 0.1% TCP-25 (0.3 mM) in HEC gel was selected for further studies (herein denoted “TCP-25 gel #1”). Antibacterial effects of the TCP-25 gel #1 on bioluminescent bacteria was quantified as described above and the result is obtained by use of luminometry shown in FIG. 3A. The TCP-25 gel #1 yielded a rapid reduction in P. aeruginosa PAO1 and S. aureus bioluminescence after only 1 minute of incubation. The antibacterial effect was mediated by bacterial permeabilization, as demonstrated by the use of a live-dead assay (BacLight Kit L 7012), which utilizes propidium iodide (red color) to detect loss of membrane integrity. Using the VCA, the bactericidal effect of the TCP-25 gel #1 on S. aureus and P. aeruginosa PAO1 was further demonstrated, yielding over 3 log reductions for the two bacteria (FIG. 3B). TCP-25 gel's effects on a series of bacterial wound isolates was analysed. TCP-25 gel #1 yielded over 3 log reductions of all clinically derived isolates of S. aureus and P. aeruginosa as well as additional wound isolates and reference strains. The results were further substantiated using TCP-25 in standard minimum inhibitory concentration (MIC) assays according to CSLA, see table 1A below:

TABLE 1A
MIC values for TCP-25 against clinical
isolates and reference strains.

| Bacteria | | MIC (μM) |
|---|---|---|
| E. coli | Clinical isolate 47.1 | 1.2 |
| | ATCC 25922 | 2.5 |
| | Clinical isolate 37.4 | 2.5 |
| | Clinical isolate 49.1 | 10 |
| P. aeruginosa | Clinical isolate 18488 | 10 |
| | Clinical isolate 62.1 | 20 |
| | Clinical isolate 10.5 | 20 |
| | Clinical isolate 15159 | 20 |
| | Clinical isolate 27.1 | 20 |
| | Clinical isolate 23.1 | 40 |
| | Clinical isolate 13.2 | 80 |
| | Clinical isolate 51.1 | 80 |
| | ATCC 27853 | 160 |
| S. aureus | Clinical isolate 18800 | 2.5 |
| | Clinical isolate 16065 | 2.5 |
| | Clinical isolate 18319 | 2.5 |
| | ATCC 29213 | 10 |
| | FDA 486 | 10 |
| | Clinical isolate 1088 | 10 |
| | Clinical isolate 1090 | 10 |
| | Clinical isolate 13430 | 10 |
| | Clinical isolate 14312 | 10 |
| | Clinical isolate 1779 | 20 |
| | Clinical isolate 2278 | 20 |
| | Clinical isolate 2279 | 20 |
| | Clinical isolate 1781 | 40 |
| | Clinical isolate 1086 | 80 |
| E. faecalis | Clinical isolate 2374 | 20 |
| S. pyogenes | AP1 | 2.5 |
| S. pneumoniae | TIGR4 | 2.5 |
| | D39 | 5 |
| | Clinical isolate PJ1354 | 20 |
| | Clinical isolate I-95 | 2.5 |
| | Clinical isolate I-104 | 5 |

The peptide was also active against a series of multi-drug-resistant isolates defined in table 1B below:

TABLE 1B
MIC values of TCP-25 against multidrug resistant bacteria

| Bacterial strains | MIC (μM) |
|---|---|
| Staphylococcus aureus ATCC 43300 - methicillin-resistant type strain | 10.3 |
| Staphylococcus aureus - methicillin-resistant clinical isolate | 10.3 |
| Staphylococcus aureus - multi-drug-resistant clinical isolate | 41.4 |
| Staphylococcus epidermidis - methicillin-resistant clinical isolate | 5.1 |
| Enterococcus faecium - vancomycin-resistant (VanA) clinical isolate | 5.1 |
| Enterococcus faecium - vancomycin-resistant (VanB) clinical isolate | 10.3 |
| Enterococcus gallinarum - vancomycin-resistant (VanC) clinical isolate | 10.3 |
| Streptococcus pneumoniae - penicillin-resistant clinical isolate | 41.4 |
| Streptococcus pneumoniae - multi-drug resistant clinical isolate | 41.4 |
| Streptococcus pyogenes - Macrolide (MLS) resistant clinical isolate | 41.4 |
| Corynebacterium jeikeium - multi-drug resistant clinical isolate | 165.7 |
| MU50 Staphylococcus aureus (MRSA) - VISA type strain | 10.3 |
| EMRSA3 Staphylococcus aureus (MRSA) - SSCmec type 1 | 5.1 |
| EMRSA16 Staphylococcus aureus (MRSA) - SSCmec type 2 | 20.7 |
| EMRSA1 Staphylococcus aureus (MRSA) - SSCmec type 3 | 10.3 |
| EMRSA15 Staphylococcus aureus (MRSA) - SSCmec type 4 | 5.1 |
| HT2001254 Staphylococcus aureus (MRSA) - PVL positive | 10.3 |
| Group G Streptococcus - macrolide-resistant clinical isolate | 165.7 |
| Enterococcus faecalis - vancomycin-resistant (VanA) clinical isolate | 41.4 |
| Enterococcus faecalis - vancomycin-resistant (VanB) clinical isolate | 5.1 |
| Enterococcus faecalis - high-level gentamicin-resistant clinical isolate | 41.4 |
| Streptococcus pyogenes- Macrolide (M-type) resistance clinical Isolate | 41.4 |
| Escherichia coli ATCC 35218 - β-lactamase positive type strain | 10.3 |
| Escherichia coli - multi-drug resistant clinical isolate | 20.7 |
| Klebsiella aerogenes - multi-drug resistant clinical isolate | 82.8 |
| Enterobacter sp - multi-drug resistant clinical isolate | 82.8 |
| Stenotrophomonas maltophila - antibiotic-resistant clinical isolate | 10.3 |
| Shigella sp - multi-drug resistant clinical isolate | 41.4 |
| Moraxella catarrhalis - β-lactamase positive clinical isolate | 2.5 |
| Moraxella catarrhalis - β-lactamase positive clinical isolate | 2.5 |
| Acinetobacter baumanii - multi-drug resistant clinical isolate | 10.3 |
| Haemophilus influenzae - β-lactamase positive clinical isolate | 165.7 |
| Citrobacter freundii - resistant clinical isolate | 165.7 |
| Escherichia coli - ESBL - TEM | 41.4 |
| Escherichia coli - ESBL - CTXM | 41.4 |
| Escherichia coli - ESBL - SHV | 20.7 |
| Klebsiella pneumoniae - ESBL - TEM + SHV | 82.8 |
| Klebsiella pneumoniae - ESBL - CTXM | 165.7 |
| Klebsiella pneumoniae - ESBL - SHV | 82.8 |

It should be noted that the MIC values in all cases were below the TCP-25 dose in the TCP-25 gel #1, which comprises 0.1% (0.3 mM). Taken together, these results demonstrate that TCP-25 retains its antibacterial activity in neutral polymers such as HPC and HEC, is active against multiple bacterial Gram-negative and Gram-positive bacterial isolates, and that the formulated TCP-25 gel #1 exerts a rapid killing effect that is mediated by bacterial permeabilization.

### Effects of TCP-25 Gel on Bacteria and Endotoxin Responses in Experimental Mouse Models

Two different animal models that simulate a situation of surgical contamination with bacteria were used, both mimicking situations of relevance for surgery and wounding. The first model, mimicking a direct and immediate contamination, TCP-25 gel #1 was inoculated with bioluminescent S. aureus and P. aeruginosa bacteria (10⁶ colony forming units, CFU/animal) and immediately injected subcutaneously in SKH1 mice. The results showed that the TCP-25 gel #1 reduced the bacteria as assessed by in vivo bioimaging, combined with analyses of CFU after 6h (FIG. 4A) and after 24h (FIG. 4E). When compared with the initial reduction of bioluminescence after 6 hours, a higher signal was recorded after 24 h, possibly due to bacterial regrowth due to the single dosage regimen. The reduction in CFU was however >2 log for the two bacteria after 24 h (FIG. 4E). To visualize the tissue distribution of TCP-25, the TCP-25 gel #1 was spiked with 1% Cy5-labeled TCP-25, and the peptide was observed to localize to the site of gel administration during the time period studied (FIG. 4A). Histology analyses of the infected tissue areas corresponding to the bacterial analyses showed an abrogated inflammatory response in the animals treated with TCP-25 gel #1 (FIG. 4B). In the second prevention model, the TCP-25 gel #1 was first injected subcutaneously in BALB/c mice. Thirty minutes later, bioluminescent S. aureus and P. aeruginosa bacteria (10⁶ CFU/animal) were injected into the site of gel deposition. As above, the results showed reductions of bacteria as assessed by in vivo bioimaging.

Control gel or TCP-25 gel #1 was injected subcutaneously, with simultaneous addition of LPS. Using mice reporting NF-κB activation, it was found that LPS, when added to the formulation yielded a local inflammatory response, which was abrogated by the gel containing TCP-25 (FIG. 4C). As above, TCP-25 gel spiked with Cy5-labeled TCP-25 were used, and it was observed that the peptide localized to the site of gel administration during the time period studied (FIG. 4C). In a separate experiment, BALB/c mice that had 6-mm polyurethane discs implanted subcutaneously to collect local wound exudates were used. This model resembles a surgical implant model, and application of LPS yielded an increase of interleukin (IL)-6 and tumor necrosis factor (TNF)-□, which were reduced upon addition of the TCP-25 gel #1 (FIG. 4D). This result is comparable to the results of the bioimaging studies (FIG. 4C). Taken together, the results demonstrated that the TCP-25 gel #1 has a significant dual anti-infective and anti-inflammatory function in vivo in subcutaneous models of infection and endotoxin-driven inflammation.

### Effects of TCP-25 Gel in a Porcine Partial Thickness Wound Model

A partial thickness wound model in Göttingen minipigs (study outlines are presented in FIG. 5A) was used. This model is translatable to the human wounding situation. In the initial 4-day study, mimicking a clinical situation where treatment is applied in connection to injury and bacterial contamination (here denoted contaminated wound model), wounds were inoculated with S. aureus, followed by application of control or TCP-25 gel #2 after a 30 min incubation time, and subsequent daily gel treatments at dressing changes. On day 4, non-treated control wounds showed visible signs of inflammation and infection (FIG. 5B). TCP-25 gel #2 treatment abrogated the infection, leading to an improved clinical score (FIG. 5B, C), reduced bacterial counts (FIG. 5D), lower IL-6 and TNF-α (FIG. 5E), and reduced inflammatory signs at the tissue level (FIG. 5F). In two separate experiments, two animals developed a superinfection on day 2, leading to a mixed infection. Wound data from these animals are presented separately here. Upon isolation and identification, bacteria responsible for superinfection was found to be P. aeruginosa. TCP-25 gel #2 also prevented this secondary, mixed infection (FIG. 5B-E). These two P. aeruginosa isolates showed sensitivity to TCP-25 in RDA and the peptide abrogated proinflammatory effects of bacterial supernatants on THP-1 cells. In order to reproduce the spontaneous superinfection with P. aeruginosa, the bacterial contamination model was repeated using S. aureus, followed by inoculation of the same wounds with one P. aeruginosa strain collected from the mixed infection above. As above, TCP-25 gel #3 treatment prevented development of infection, leading to an improved clinical score, reduced bacterial counts, and lower TNF-α and IL-1. These experiments were followed by a 10-day study, where wounds were inoculated with S. aureus as above, followed by daily treatments for 3 days, and thereafter every second day. Also here, TCP-25 gel #2 treatment prevented S. aureus infection, with improved wound status (FIG. 5G). Histological analysis showed that peptide-treated wounds were completely re-epithelialized, which was not observed in the infected untreated wounds (FIG. 5G). Finally, to evaluate effects on normal healing of uninfected wounds, TCP-25 gel #2 was similarly applied for 10 days on the partial thickness wounds in a separate experiment. Both control gel and TCP-25 gel-treated wounds showed normal wound healing with no signs of tissue toxicity (FIG. 5H).

### TCP-25 Gel Pharmacokinetics In Vitro and In Vivo

Pharmacokinetics of TCP-25 in the gel formulation in vitro and in an in vivo model. In vitro, the diffusion rate of the TAMRA-labeled TCP-25 from the gel to a buffer solution was analysed (FIG. 10A). For the analysis a TCP-25 gel comprising 0.1% TAMRA-TCP-25, 10 mM Tris, pH 7.4, 1.3% glycerol, and 1.5% HEC was used. In the two-compartment system used, the peptide was eluted gradually, with no observed initial burst, from the gel phase as determined by the fluorescence readings. The peptide was detected in the buffer compartment after 2 hours, and about half of the peptide amount was released from the hydrogel after 6 hours, data compatible with the weak peptide-polymer interactions detected by CD (FIG. 2).

To assess the distribution of TCP-25 in vivo, the TCP-25 gel #1 (spiked with Cy5-labeled TCP-25) was subcutaneously deposited in the dorsum of SKH1 mice. Longitudinal IVIS bioimaging was used to track the diffusion of the peptide from the gel into the surrounding tissues, and these results showed that TCP-25 was largely retained at the injection site (FIG. 10B). Thus, the results were compatible with the slow diffusion observed in the in vitro model (FIG. 10A). The presence of LPS did not influence the distribution of TCP-25 in this in vivo model (FIG. 10B, lower row). To assess possible peptide uptake through the skin and wounds, TCP-25 gel #2, #3 and #4, spiked with Cy5-labeled TCP-25 as above, was applied either onto intact porcine skin or onto wounds ex vivo and in vivo (FIG. C, D). The total peptide concentration was kept at 0.1% (TCP-25 gel #2), but also increased to 1% (TCP-25 gel #3) in vivo (FIG. 10C), and 2% (TCP-25 gel #4) ex vivo (FIG. D). The fluorescent peptide remained locally at the application site and no visible uptake was observed through skin or wounds into the underlying tissues. To assess possible systemic uptake of TCP-25 after topical treatment, plasma from the partial thickness wound models was analyzed using mass spectrometry (FIG. 10E). No TCP-25 peptide was detected in the porcine plasma after a 24-h application of TCP-25 gel #2 on partial thickness wounds. However, intact TCP-25 was detected in wound fluids from dressings obtained from infected and uninfected wounds after a 24-h treatment period.

Degradation of TCP-25 by Neutrophil Elastase In Vitro and Comparison with Proteolytic TCP Fragments Generated In Vitro and In Vivo

Multiple TCPs are generated by proteolytic digestion of thrombin in vitro by the major protease human neutrophil elastase (HNE), a dominant enzyme active during wound healing and inflammation. The corresponding C-terminal peptide sequences were identified in wound fluids from acute and non-healing ulcers, and among these were the TCP fragments FYTHVFRLKKWIQKVIDQFGE and HVFRLKKWIQKVIDQFGE, SEQ ID Nos 2 and 4. The digestion pattern of TCP-25 after it was subjected to HNE was determined. Enzyme digestion was performed for different time periods, and the fragmentation was evaluated by LC-MS/MS. FIG. 6A shows the major peptides that were obtained after digestion for different time periods. All the TCP-25 fragments that were identified are presented graphically in FIG. 6B, and TCP fragments were compared to fragments found after thrombin digestion with HNE. TCPs detected in wounds in vivo are also shown in FIG. 6B. The results show that multiple peptides detected in wound fluid, as well as after digestion of thrombin with HNE overlap structurally with those identified after HNE digestion of TCP-25 (FIG. 6B). For example, the peptide HVFRLKKWIQKVIDQFGE (HVF18) (SEQ ID NO; 4) was detected after proteolysis of TCP-25 by HNE, as well as the major fragment GKYGFYTHVFRLKKWIQKVI (GKY20) (SEQ ID NO 3) (FIG. 6A). The digestion patterns were also similar in the buffer and in the HEC formulation (FIG. 6C). The generated peptide fragments retained their antibacterial activities for digestion periods of up to 6 hours in the RDA, although longer digestion times led to a reduction of peptide activity particularly when RDA was performed at physiological salt conditions (0.15 M NaCl) (FIG. 6D). In summary, the results show that HNE degradation of TCP-25 resulted in the generation of a multitude of bioactive TCP-fragments, of which several overlapped with identical peptides generated from human thrombin and were also present in human wounds in vivo.

### Stability of TCP-25 In Vitro

In contrast to the rapid degradation of TCP-25 when subjected to HNE, the TCP-25 did not show changes when stored either in buffer or in the HEC formulation (TCP-25 gel #1) for extended periods of time at 4° C. or 20° C. Mass spectrometry analysis using MALDI-mass spectrometry found no indication of degradation or oxidation/deamination for up to 180 days at these temperatures. However, storage for 180 days at 37° C. resulted in mass changes. Activity assays corresponded well with the mass analyses and showed that the peptides' antibacterial as well as immunomodulatory effects were preserved after storage for up to 180 days. It has been previously shown for other peptides that their half-life in plasma depends on the species used for plasma collection, possibly due to differences in endoproteinases or other factors affecting stability. Hence, the stability of TCP-25 was studied in human, porcine, and mouse plasma using mass spectrometry. TCP-25, when incubated at 37° C., was stable when incubated in phosphate buffered saline (PBS) and mouse plasma. In human and mini pig plasma, the half-lives were calculated as 8.1 and 2.5 h, respectively.

Comparison of TCP-25 Hydrogel with Clinically Used Wound Treatments and Effects on Established Infection

Various silver dressings and products containing PHMB are commonly used to prevent infections on burns or surgical wounds, or as treatments for chronic leg ulcers. Thus, in the next study, using the previous treatment scheme for the contaminated wound model, TCP-25 gel #3 was compared with the common wound treatments Mepilex Ag and Prontosan gel, which contain silver and PHMB, respectively. Thus, wounds were inoculated by S. aureus and then treated with either of the three wound treatments. As demonstrated previously for 0.1% TCP-25 gel (TCP-25 gel #2), the 1% TCP-25 gel (TCP-gel #3) treatment used in this study also prevented S. aureus infection (FIGS. 7A and B) without any observed negative effects in this 4-day contaminated wound model, as assessed by clinical scoring (FIG. 7C) and histological analysis (FIG. 7D). We observed, however, that Mepilex Ag, was not effective in preventing S. aureus infection, because the clinical status and wound bacterial numbers were similar to those for the infected control (FIG. 7A-D). Although a reduction of bacteria in the Mepilex Ag dressing extracts was observed, this change was not statistically significant (P>0.05) (FIG. S11A). In contrast, TCP-25 gel #3 was able to reduce (>5 log) bacterial numbers in the dressing extracts. In agreement with this observation, in vitro, we found that the Mepilex Ag dressing, while showing antibacterial effects against S. aureus in buffer conditions was not effective in presence of plasma and wound fluid. In this infection model, which was mainly aimed at evaluating antibacterial effects, Prontosan treatment yielded similar results to TCP-25 gel #3 on clinical wound scoring, reduction of bacterial infection, and TNF-α (FIGS. 7A-D and FIG. 7L). Finally, in order to evaluate the effect of TCP-25 in a model of established infection, wounds were inoculated with S. aureus, followed by establishment of infection for 24 hours, and subsequent treatment with 1% TCP-25 gel (TCP-25 gel #3) or Prontosan (FIG. 7E). Mepilex Ag was omitted since it was found ineffective in the contaminated wound model above. Before initiation of treatment, at day 2, all wounds showed clinically visible signs of infection and similar bacterial numbers (FIGS. 7F and G). Treatments were applied daily for 2 consecutive days, and then every second day until day 10. The results showed that 1% TCP-25 gel #3 treatment indeed reduced S. aureus infection and related inflammation as assessed by bacterial numbers (FIG. 7G), and lowered concentrations of cytokines TNF-α (FIG. 7H) and IL-1P (FIG. 7M). Notably, reductions in cytokine concentrations preceded the observed antibacterial effects in the TCP-25 gel #3 treated wounds (FIG. 7H and FIG. 7M). Thus, results obtained at day 3 (after 24 hours of treatment with TCP-25 gel #3), show reductions of TNF-α(FIG. 7H) and IL-1P (FIG. 7M) in spite of bacterial numbers in the wounds similar to those of the control. Although we observed an initial effect on bacterial numbers by Prontosan at day 5, it was overall not effective in reducing S. aureus infection and inflammation in the model of an established infection (FIGS. 7F-H and FIG. 7M). Taken together, these results demonstrate that TCP-25 gel #3 is effective in targeting S. aureus in models of contaminated as well as infected wounds, and that the treatment has a capacity of reducing cytokines independently of bacterial presence.

Although the porcine wound models above indicated that TCP-25 can target bacteria as well as inflammation, we wanted to separately evaluate the latter aspect of the TCP-25 gel concept further. In a mouse model of subcutaneous infection, Prontosan gel was equally as effective as TCP-25 gel #3 in reducing S. aureus and P. aeruginosa (FIG. 7I), which was consistent with the observed preventive effects of both treatments in the S. aureus contaminated wound model (FIGS. 7A and B). To specifically assess possible anti-endotoxic effects, we next injected LPS subcutaneously, either with addition of Prontosan gel, or 0.1% TCP-25 gel (TCP-25 gel #2) on each side of the mouse dorsum. In this model, TCP-25 gel #2 exhibited a significant (P<0.01) anti-inflammatory activity, when compared to Prontosan gel (FIG. 7J). In agreement with these in vivo data, in vitro experiments showed that TCP-25 exerted higher LPS-quenching effect compared to PHMB (FIG. 7K). Finally, a single-dose toxicity study was performed. Mice were subcutaneously injected with 5 mg TCP-25 and organs were collected after 24 h. Histology of the lung, kidney, liver, spleen, and skin did not show any signs of toxicity.

### TCP-25 Targets Inflammation in Wounds

Treatment with TCP-25 gels could also target TLR-mediated inflammation related to wound infection in general. Initial experiments demonstrated that wound fluid derived from the mixed infection wounds described above (FIG. 5D) induced NF-κB activation in the THP-1 cell model system, whereas such induction was not observed for either TCP-25 gel-treated wounds or uninfected wounds (FIG. 8A). For this experiment TCP-25 gel #5 comprising 0.1% TCP-25, 2% HEC, 1.3% glycerol and 10 mM Tris HCl at pH 7.4 was used. However, because this absence of NF-κB induction could be ascribed to the anti-infective effects of the treatment, we subsequently added TCP-25 to wound fluids derived from the animals infected with both S. aureus and P. aeruginosa. In this case, TCP-25 reduced the wound fluid-induced inflammation (FIG. 8B). Patients with non-healing venous ulcers are commonly colonized or infected by S. aureus and P. aeruginosa. Wound fluids derived from five patients with wounds infected by these bacteria were selected and found to activate THP-1 cells to varying degrees (FIG. 8C). In this complex environment, adding TCP-25 reduced the NF-κB activation, as noted above (FIG. 8B). Taken together, the results indicate that TCP-25 also has the potential to attenuate inflammation in complex wound environments containing endotoxins and other TLR agonists and cytokines.

### Rheology

The rheological properties of TCP-25 gels were measured. Gel strengths of 2% HEC gel without (control), or with 0.1 or 1% TCP-25 (TCP-25 gel #2 and TCP-25 gel #3) were analyzed on a Kinexus Pro rheometer (Malvern Panalytical Ltd., Malvern, UK) equipped with a plate-plate geometry and a gap of 1 mm. A shear strain from 0.001 to 10 strain was applied and the linear viscoelastic region (LVR), the storage modulus (G′) and the loss modulus (G″) was determined at 1 Hz frequency and 25° C. The results are shown in FIG. 9.

The flow point was determined using a Kinexus Pro rheometer as the shear stress (Pa) at the crossover point between G′ and G″. The following results were obtained:

TABLE 2

| Hydrogel | Flow point |
|---|---|
| Control gel (2% HEC) | 48.8 Pa |
| TCP-25 gel#2 | 53.8 Pa |
| TCP-25 gel#3 | 51.9 Pa |

### Discussion

Infectious diseases account for millions of deaths worldwide each year. In the wound and surgical areas, infected burn wounds and postoperative infections cause significant morbidity and are associated with a risk of deadly systemic complications such as sepsis. The decreasing effectiveness of antibiotics and other antimicrobial agents because of resistance development is an increasing problem. We have reached a point for certain infections where therapeutic agents are no longer available. There is, therefore, an important and unmet need for new treatments that will improve healing and reduce infection and inflammation in various types of wounds. Current therapies using systemic antibiotics or local antiseptics only target the bacteria without any effects on the associated inflammation. Conversely, therapies reducing the excessive inflammatory responses in wounds mainly target matrix metalloproteinases (MMPs) or locally produced cytokines, and the latter is currently at the preclinical testing stage. The present invention demonstrate an alternative approach based on a hydrogel incorporating a peptide targeting bacteria and the proinflammatory products that are released. As illustrated in FIG. 8D, such a “dual-action” gel has antiseptic functions and targets the proinflammatory responses by blocking bacterial products such as endotoxins. TCP-25 acting upstream of NF-κB also distinguishes it from previous concepts that target MMPs and cytokines, which are downstream of the NF-κB-mediated response.

The invention demonstrates that TCP-25 hydrogels effectively kills pathogens, such as S. aureus and P. aeruginosa in vitro and in vivo. Staphylococci are a major cause underlying postoperative surgical infections, and emerging multi-drug-resistant strains complicate the treatment possibilities. Because the TCP-25 mode of action is different from existing antibiotics, its capability of targeting MRSA in vitro is of interest because it could reduce the risk of infections with resistant staphylococci. The MIC analyses for TCP-25 showed that the peptide had comparable activity to other antibacterial peptides such as LL-37 and omiganan, which are in clinical development. Similarly, as shown here, TCP-25 also killed a series of P. aeruginosa and S. aureus isolates in vitro when formulated in a gel. The data from the in vivo bioimaging studies using mouse models mimicking situations of acute wounding and bacterial contamination, demonstrate that TCP-25 hydrogels also has the ability to prevent subcutaneous S. aureus and P. aeruginosa infection in vivo. Of particular importance was the finding that TCP-25 hydrogeld also reduced local responses to subcutaneously injected endotoxins in the experimental mouse models.

Porcine wound healing studies are considered to have a good translation to the human wound healing situation. In the short-term partial thickness model of a contaminated wound, the TCP-25 hydrogels were efficient in preventing infection after inoculation of the wounds with S. aureus. In two separate experiments, animals acquired a superinfection on day 2 with P. aeruginosa, and the TCP-25 gel treatment prevented such a mixed infection. Notably, after isolation and characterization of the P. aeruginosa strain, this was successfully reproduced by contaminating the wounds with such P. aeruginosa bacteria 24 hours after S. aureus inoculation.

In these above experiments, a reduction in the cytokine concentrations was regularly observed after TCP-25 gel treatment. The observation that the proinflammatory effects of the wound exudates derived from infected partial thickness wounds could also be blocked by exogenously added TCP-25 shows that the peptide has the capacity to reduce bacteria-induced inflammation during wounding in vivo. Indeed, these findings are compatible with the initial experiments demonstrating that TCP-25 hydrogels reduce endotoxin-induced tissue inflammation in experimental mouse models. Importantly, TCP-hydrogels were also effective in a model of established S. aureus infection. In this model cytokine concentrations were reduced before bacterial numbers were affected, lending further support for TCP-25 hydrogel's anti-inflammatory action in vivo.

All these observations are of clinical relevance because large patient groups with non-healing ulcers of various etiologies, such as diabetes and arterial or venous insufficiency, have an inhibited wound healing process. The latter group is the largest and is particularly characterized by chronic and dysfunctional inflammation with high levels of proinflammatory factors, proteases, and bacteria. The observation that TCP-25 also reduces pro-inflammatory monocyte responses to wound fluids derived from patients with non-healing venous ulcers that are infected with S. aureus and P. aeruginosa is therefore highly relevant from a clinical perspective, as it indicates that the peptide has the potential to target excessive inflammation in wounds that contain a mixture of various TLR agonists. This observation is also consistent with the observation that TCP-25 targets endotoxins and other TLR agonists such as lipoteichoic acid, peptidoglycan, and CpG DNA.

To further illustrate the clinical and translational potential of the “dual action” gel concept of the invention, the efficacy of TCP-25 hydrogels was compared with two commonly used wound treatments, Mepilex Ag and Prontosan wound gel. The intended use for both treatments is to treat wounds such as burns and non-healing ulcers. The silver-containing dressing did not prevent S. aureus infection, which was unexpected given the widespread use of silver as an antiseptic in various wound indications. However, despite its long history and common usage, there is little clinical evidence demonstrating that silver-containing dressings or creams improve wound healing or prevent infection. Both TCP-25 hydrogels and Prontosan were antibacterial in the mouse model of subcutaneous bacterial infection and in the porcine partial thickness S. aureus contaminated wound model. Interestingly, contrasting to the contaminated wound model, Prontosan overall neither reduced bacterial infection nor cytokines in the model of established S. aureus infection. Of note is that this observation indeed corresponds to recent findings on surgical wounds demonstrating that dressings soaked with Prontosan solution were not effective in reducing bacterial numbers and infection. Finally, studies using the NF-κB reporter mouse model demonstrated that the endotoxin scavenging effects were unique for the TCP-25 hydrogels, further illustrating the functional differences between PHMB and TCP-25.

Previous studies have addressed structure function relationships of TCP-25 and its bioactive epitopes. For example, HVF18 (HVFRLKKWIQKVIDQFGE)(SEQ ID NO:4) is present in wound fluids in vivo. The antibacterial and LPS binding epitope of TCP-25 is also present in GKY20 (GKYGFYTHVFRLKKWIQKVI) (SEQ ID NO 3), which contains the first 20 amino acids of TCP-25. Similar to TCP-25, both of these peptides were shown to exert dual antibacterial and anti-inflammatory effects, and they also reduce mortality in mouse models of endotoxic shock. Using a screening-based approach, GKY20 was found to display an improved therapeutic index because this peptide retained its anti-infective capacity while showing less hemolysis in human blood. The findings that fragments such as HVF18, and related truncations of TCP-25 are present in vivo in wound fluids illustrate a concept of redundancy, with multiple bioactive peptide fragments that are simultaneously present. It has been increasingly appreciated that a multitude of transient, biological interactions (K_{d}>μM) occur frequently in biological systems. Sharing many characteristics with “transient drugs”, the TCP family, with its multiple interactions and affinities in the μM range, therefore represents an elegant example of such an endogenous biological system that modulates the host responses to infection.

From a pharmaceutical perspective, it was, therefore, of interest to explore whether similar TCP fragments as those that are present in wounds in vivo could be generated from synthetically produced TCP-25. TCP-25 was cleaved by HNE, a major enzyme that is active during normal wound healing, and notably, HVF18 was identified as a major bioactive peptide metabolite. It was also interesting that one of the other major fragments was identical to the previously described GKY20 peptide (FIG. 7). Mass spectrometry analyses also identified a series of other truncated TCP-25-derived fragments that were previously described in wounds in vivo, of which several have been shown to retain both antibacterial and anti-endotoxic effects in vitro. Additionally, RDA assays demonstrated that cleaved TCP-25 retains antibacterial activity, and a reduction in activity was particularly noted in the presence of physiological salt conditions, which is compatible with previous observations that shorter TCP fragments exhibit reduced salt resistance. However, the data indicate that activity of the TCP fragments are also retained after digestion periods for up to 3-6 hours. A comparison between the degradation profiles of pure TCP-25 and TCP-25 in hydrogel identified similar peptide fragments, indicating that the formulation polymer did not interfere with the degradation patterns obtained. Thus, these data demonstrate that upon proteolysis, TCP-25 may release several bioactive fragments with retained transient interactions and “dual action” functionalities in vivo, motivating the use of TCP-25 as an active drug, which in turn can act as a precursor for bioactive peptides such as the previously defined N- and C-terminally truncated TCP-25 variants HVF18 and GKY20, respectively. In contrast to the rapid and specific degradation by neutrophil elastase, TCP-25 was highly stable for up to 180 days at room temperature in both the buffer and the hydrogel formulation, which is relevant for translation into therapy and clinical use. Additionally, the TCP-25 gel was retained to a high degree at the site of injection in the mouse models and at the wound and skin surface in the porcine models with no systemic absorption of TCP-25.

From a drug delivery perspective, the selection of clinically and pharmaceutically acceptable formulations that enable the dual function of TCP-25 is not trivial because this peptide, as well as the fragments HVF18, GKY20, and FYT21, all require a flexible conformation that enables critical bacterial and host cell interactions. To study the influence of formulations on TCP-25, we utilized a combination of both bioassays and structural analysis using CD. The analyses showed that neutral hydrogels enabled TCP-action, whereas anionic CMC and the micelle-forming F127 were inhibitory to variable degrees. Additionally, the endotoxin-scavenging capacity was particularly sensitive to inhibition by CMC. Structural clues about this observation were obtained using CD, which showed that the peptide assumed an ordered conformation, involving helical structure induction, particularly in the presence of CMC, whereas it was unordered in the presence of HEC. The observed conformational change in presence of CMC is compatible with interactions between TCP-25 and the anionic polymer. As demonstrated by the bioassays, the peptide scavenging effect by this polymer, in turn, reduces TCP-25 binding to bacteria and LPS. A similar reasoning may be applied on the pluronic, which has hydrophobic polyoxypropylene units between hydrophilic units of polyoxyethylene, likely enabling binding to the amphipathic TCP-25. Thus, based on these structural and functional analyses, it was concluded that TCP-25 require a non-interacting carrier formulation, such as HEC/HPC, enabling the peptide's direct interactions with target bacteria and host cells. The rheology measurements of flow point indicate that the peptide does not modify the gel characteristics (FIG. 9A and Table 2). These results are compatible with those presented in FIG. 2 indicating that the peptide does not interact significantly with the HEC polymer. Furthermore, as G′<G″, at low strain, all formulations display gel-like characteristics (FIG. 9B).

### Example 2

In example 2 the Minimal inhibitory concentration (MIC) and Minimal bactericidal concentration (MBC) was determined for S. aureus, P. aeruginosa, and E. coli when treated with TCP-25 (uM) in combination with EDTA (mM) in either a 10 mM Tris buffer, pH 7.4. n=3, or a 10 mM Sodium Acetate buffer, pH 5. n=3.

The MIC was determined essentially as described herein above in Example 1. The MBC indicates the minimal concentration of TCP-25 capable of killing the bacteria. MBC was determined in essentially the same manner as MIC except that the MBC was taken as the concentration where a decrease in bacterial load was observed.

Table 3 shows the results for the 10 mM Tris buffer, pH 7.4.

| | EDTA (mM) | | | | | | | | | | | |
|---|---|---|---|---|---|---|---|---|---|---|---|---|
| | 10 | 10 | 5 | 5 | 2.5 | 2.5 | 1 | 1 | 0.5 | 0.5 | 0 | 0 |
| TRIS pH 7.4 | MIC | MBC | MIC | MBC | MIC | MBC | MIC | MBC | MIC | MBC | MIC | MBC |
| S. aureus | ≤1.25 | 10 | ≤1.25 | 10 | ≤1.25 | 10 | 2.5 | ≥160 | 40 | ≥160 | 40 | ≥160 |
| P. aeruginosa | ≤1.25 | 10 | ≤1.25 | 10 | ≤1.25 | 10 | 10 | ≥160 | 40 | ≥160 | 40 | ≥160 |
| E. coli | ≤1.25 | 5 | ≤1.25 | 5 | ≤1.25 | 5 | 10 | ≥160 | 40 | ≥160 | 40 | ≥160 |

Table 4 shows the results for the 10 mM Sodium Acetate buffer, pH 5.

| | EDTA (mM) | | | | | | | | | | | |
|---|---|---|---|---|---|---|---|---|---|---|---|---|
| | 10 | 10 | 5 | 5 | 2.5 | 2.5 | 1 | 1 | 0.5 | 0.5 | 0 | 0 |
| NaOH pH 5 | MIC | MBC | MIC | MBC | MIC | MBC | MIC | MBC | MIC | MBC | MIC | MBC |
| S. aureus | ≤1.25 | ≤1.25 | ≤1.25 | ≤1.25 | ≤1.25 | ≤1.25 | 2.5 | 10 | 10 | 40 | 20 | ≥160 |
| P. aeruginosa | ≤1.25 | ≤1.25 | ≤1.25 | ≤1.25 | ≤1.25 | ≤1.25 | 2.5 | 10 | 10 | 40 | 40 | ≥160 |
| E. coli | ≤1.25 | ≤1.25 | ≤1.25 | ≤1.25 | ≤1.25 | ≤1.25 | ≤1.25 | ≤1.25 | 2.5 | 10 | 40 | ≥160 |

As shown by table 3 and 4 the addition of EDTA provides a significantly decreased MIC value and a significantly decreased MBC value compared to the TCP-25 peptide alone (EDTA=0 mM)

### Example 4

Solubility of TCP-25 was determined based on visual inspection. Solutions comprising 0.1% TCP-25, 2.5 mM EDTA and either 1.9 or 2% glycerol were prepared. In addition said solutions also comprised either TRIS (pH 7.4) or Acetate (pH 5.0). The results are shown in FIG. 11.

Visual inspection of the solutions show that that at low pH (pH 5.0), in Acetate buffer (10 mM or 25 mM) the solutions are significantly less cloudy compared to the solutions at high pH (pH 7.4) in Tris buffer (10 mM or 25 mM). This indicates increased solubility of TCP-25 at pH 5.0 compared to at pH 7.4.

### Example 5

The formation of biofilms can hamper the effect of antibacterial treatments. Therefore, the efficacy of TCP-25 EDTA combination in Tris and Acetate based gels against biofilms was assessed.

The gels comprising the components indicated in Table 5 were prepared essentially as described in Example 1.

TABLE 5

| | TCP-25 gel | | | | | | | |
|---|---|---|---|---|---|---|---|---|
| | #6 | #7 | #8 | #9 | #10 | #11 | #12 | #13 |
| Tris pH 7.4 | 10 mM | 25 mM | 10 mM | 25 mM | — | — | — | — |
| Acetate pH 5 | — | — | — | — | 10 mM | 25 mM | 10 mM | 25 mM |
| EDTA | — | — | 2.5 mM | 2.5 mM | — | — | 2.5 mM | 2.5 mM |
| Glycerol | 2.0% | 1.9% | 2.0% | 1.9% | 2.0% | 1.9% | 2.0% | 1.9% |
| HEC | 2.0% | 2.0% | 2.0% | 2.0% | 2.0% | 2.0% | 2.0% | 2.0% |
| TCP-25 | 0.1% | 0.1% | 0.1% | 0.1% | 0.1% | 0.1% | 0.1% | 0.1% |

Control gels without TCP-25 were also prepared.
5 μl of S. aureus (ATCC 29213) (10⁵ CFU/ml) were incubated at 37° C. on 96-well flexible vinyl plates in 100 μl of growth medium (0.5% TSB+0.2% glucose) for the duration of 48 hours to establish a mature biofilm. After biofilm maturation, the planktonic cells were removed and 100 μl of each of the gels described in Table 4 were added to the wells and incubated for 2 hours at 37° C. After incubation the biofilm was disrupted, bacteria plated and CFU was determined. The results are shown in FIG. 12. TCP-25 has some activity against the biofilm, however the combination of TCP-25 and EDTA is very effective against the biofilm.

5 μl of P. aeruginosa (PAO1) (10⁵ CFU/ml) were incubated at 37° C. on 96-well flexible vinyl plates in 100 μl of growth medium (1×M63) for the duration of 48 hours to establish a mature biofilm. After biofilm maturation, the planktonic cells were removed and 100 μl of each of the gels described in Table 4 were added to the wells and incubated for 2 hours. After incubation the CFU was determined as above. The results are shown in FIG. 13.

For S. aureus (FIG. 12), complete inhibition was achieved when a combination of Tris (10 or 25 mM), EDTA and TCP-25 was used. The same effect was achieved by using the combination of Acetate (10 mM or 25 mM), EDTA and TCP-25. For P. aeruginosa (FIG. 13), complete inhibition was as well achieved when a combination of Tris (10 mM or 25 mM), EDTA and TCP-25 was used. A strong inhibitory effect was also seen for the combination of Acetate (10 mM or 25 mM Acetate), EDTA and TCP-25.

### Example 6

The anti-bacterial effect of various TCP-25 gels were tested on a pig skin ex vivo model. TCP gels comprising the components indicated in Table 6 were prepared essentially as described in Example 1.

TABLE 6

| | TCP-25 gel | | | | |
|---|---|---|---|---|---|
| | #14 | #15 | #16 | #17 | #18 |
| Tris pH 7.4 | 10 mM | — | — | — | — |
| Acetate pH 5.0 | — | 25 mM | 25 mM | 25 mM | 25 mM |
| EDTA | — | 2.5 mM | 5 mM | 10 mM | 20 mM |
| Glycerol | 2.0% | 1.9% | 1.9% | 1.9% | 1.9% |
| HEC | 2.0% | 2.0% | 2.0% | 2.0% | 2.0% |
| TCP-25 | 0.1% | 0.1% | 0.1% | 0.1% | 0.1% |

Pig skins were stored in the freezer. Before use, frozen skins were thawed and washed with ethanol (70%) and sterile water. Wounds of a standardized size were created on skins using a thermal device. On a petri dish, skins were kept partially submerged in PBS to retain their moisture. Wounds on the ex vivo pig skin were infected with 30 μl of bacterial solution (Pseudomonas aeruginosa, 10⁸ CFU/ml) and incubated for 2 hours at 37° C. prior to addition of treatments. 100 μl of each of the TCP-25 gels described in Table 6 were applied on a wound and incubated for another 2 hours at 37° C. CFU on the surface of the burn wound or in the burn wound tissue was determined. Infected but untreated pig skin was used as control.

The results are shown in FIG. 14. Gels comprising 0.1% TCP-25 and 20 mM of EDTA were the most effective in reducing the bacterial load.

### Example 7

When using a formulation based on hydroxyethylcellulose, glycerol, and Tris-buffer at pH 7.4, an unexpected turbidity of the hydrogel was observed, particularly when using the peptide at higher concentrations (0.3 mM). With this observation as background, the underlying mechanisms of the observed turbidity were investigated.

It was shown that at pH 7.4, the peptide assumes a dose-dependent increase in α-helical structure. Such helical induction, indicative of self-interactions is not observed at pH 5.0. Intrinsic tryptophan fluorescence, shows that TCP-25 is more stable at higher concentrations (0.3 mM), when exposed to high temperatures or high concentration of denaturant agents, which is compatible with oligomer formation. Moreover, analysis by dynamic light scattering (DLS) demonstrates that the oligomerization of TCP-25 is highly dynamic, and depending on pH, time and temperature.

### Materials and Methods

### Peptide:

The thrombin-derived peptide “TCP-25” (GKYGFYTHVFRLKKWIQKVIDQFGE)(SEQ ID NO:1) was synthesized by AmbioPharm, Inc. (USA). The purity (over 95%) was confirmed by mass spectral analysis (MALDI-TOF Voyager, USA).

Turbidity assay: TCP-25 was resuspended in 10 mM Tris at pH 7.4 or in 10 mM NaOAc at pH 5 and 5.8 at increasing concentrations (10-300 μM) and incubated for 1 h at RT. Then, the turbidity was monitored by measuring the absorbance and transmittance at 405 nm using a DU® 800 UV/Visible Spectrophotometer (Beckman Coulter™, USA).

Electrophoresis and Western blot: TCP-25 was resuspended in 10 mM Tris pH 7.4 or in 10 mM NaOAc at pH 5 and 5.8, at a concentration of 1 mM. Thirty μL of the respective samples were then centrifuged at 14 000 g for 20 min. Ten μL of the supernatant and the complete pellet were loaded on 10-20% Novex Tricine pre-cast gel from Invitrogen (USA). Electrophoresis was performed at 100 V for 100 min. The gel was stained by using Coomassie Brilliant blue (Invitrogen, USA), and images were obtained using a Gel Doc Imager (Bio-Rad Laboratories, USA). For analysis of oligomerisation, a concentration range of TCP-25 (10-300 μM in 10 μL) was loaded on BN-PAGE (NativePAGE Bis-Tris Gels System 4-16%, Invitrogen) according to the manufacturer's instructions. For Western blotting, the material was subsequently transferred to a PVDF membrane using the Trans-Blot Turbo (Bio-Rad, USA). Polyclonal rabbit antibodies against the C-terminal thrombin epitope VFR17 (VFRLKKWIQKVIDQFGE; diluted 1:1000, Innovagen AB, Sweden), followed by porcine anti-rabbit HRP conjugated antibodies (1:1000, Dako, Denmark), were used to detect TCP-25. The peptide was visualized by incubating the membrane with SuperSignal West Pico Chemiluminescent Substrate (Thermo Scientific, Denmark) for 5 min followed by detection using a ChemiDoc XRS Imager (Bio-Rad).

Circular dichroism spectroscopy: Circular dichroism (CD) was used to analyze the change in secondary structure of TCP-25 at different concentrations (10-300 μM) and in different buffer systems (10 mM Tris pH 7.4, 10 mM NaOAc pH 5 and 5.8). The measurements were performed on a Jasco J-810 spectropolarimeter (Jasco, USA) equipped with a Jasco CDF-426S Peltier set to 25° C. Quartz cuvettes (0.1 and 0.2 cm) (Hellma, GmbH & Co, Germany) were used for TCP-25 concentrations of 100-300 μM and 10-30 μM, respectively. The spectra were recorded between 190-260 nm (scan speed: 20 nm/min) as an average of 5 measurements. Raw spectra were corrected for buffer contribution and converted to mean residue ellipticity, θ (mdeg cm² dmol⁻¹). Estimation of the secondary structure was carried out according the equation reported by Morrissette et al. 1973.

Transmission electron microscopy: The oligomers of TCP-25 were visualized by transmission electron microscopy (TEM) (Jeol Jem 1230; Jeol, Japan) in combination with negative staining. In particular, 10 and 300 μM TCP-25 (corresponding to 0.003 wt % and 0.1 wt %, respectively) dissolved in 10 mM Tris pH 7.4 or in 10 mM NaOAc pH 5 were analyzed. After dissolving, 5 μL of each sample were adsorbed onto carbon coated grids (Copper mesh, 400) for 60 s and stained with 7 μl of 2% uranyl Acetate for 30 s. The grids were rendered hydrophilic via glow discharge at low air pressure. Analysis was done on view fields (magnification ′4200) of the mounted samples on the grid (pitch 62 μm) from three independent experiments.

Chemical crosslinking: Twenty μL of 1 mM TCP-25 dissolved in 20 mM HEPES were incubated with increasing concentrations of BS³ (from 18-580 μM) for 30 min at RT. The crosslinking reaction was terminated by addition of 1 μL of 1 M Tris pH 7.4. The oligomers formed were analyzed on 10-20% Novex Tricine pre-cast gel from Invitrogen (USA) followed by Coomassie staining as described above.

High pressure liquid chromatography (HPLC): Peptide samples crosslinked with 145 and 580 μM of BS³ were further characterized by reverse-phase chromatography on a Phenomenex Kinetex C18-column (50×2.1 mm 2.6 μM, 100 Å pore size, California, USA) by using the Agilent 1260 Infinity System. The column was equilibrated using 95% of buffer A containing 0.25% of TFA in MilliQ and 5% of Buffer B containing 0.25% of TFA in acetonitrile. The peptide with or without crosslinker was dissolved in Buffer A (1:3), and 3 μg were injected onto the system. The elution profile was monitored during the gradient (35% of B at 5 min, 45% at 10 min) and the spectrum at 215 nm was recorded. The flow rate of the column was 0.5 mL/min and all runs were performed at RT.

Thermal and chemical denaturation: Thermal and chemical denaturation were analyzed by recording the emission fluorescence spectra between 300-450 nm, following excitation at 280 nm. The intrinsic fluorescence of 10 and 300 μM TCP-25 (corresponding to 0.003 wt % and 0.1 wt %, respectively), dissolved in 10 mM Tris pH 7.4 or 10 mM NaOAc pH 5 respectively, was measured in a 10-mm quartz cuvette by using a Jasco J-810 spectropolarimeter equipped with a FMO-427S fluorescence module, with a scan speed of 200 nm/min and 2 nm slit width. Thermal denaturation was induced by increasing the temperature (20 to 100° C.). The peptide was incubated at the desired temperature for 10 min before taking the measurements. Tₘ was determined by fitting the maximum emission fluorescence as a function of increasing temperature. Chemical denaturation was performed by incubating the peptide at 4° C. for 24 h with increasing concentrations (0-5 M) of urea or guanidinium chloride (Gnd-HCl) before measuring the intrinsic fluorescence. Then Cₘ was calculated reporting the fluorescence ratio (F₃₃₇/F₃₅₀) as a function of the concentration of the chemical agent. The results are expressed as an average of three independent experiments±SEM.

Dynamic light scattering: The size of oligomers of TCP-25 and their relative concentrations in the solution was determined by using Zetasizer Ultra system (Malvern Panalytical, UK), using quartz cuvette with a final volume of 75 μL. The TCP25 peptide was dissolved in 10 mM Tris pH 7.4 or in 10 mM NaOAc pH 5 at 300 μM concentration immediately before the first data acquisition. The oligomerization rate was monitored at different time points (0-24 h and after 1 week) and after storage at different temperatures (RT, 4 and −20° C.). For the peptide stored at −20° C., time 0 refers to the reading immediately after melting. All the reads were taken at 25° C. Data was processed using Zetasizer Ultra-Pro ZS Xplorer software version 1.31. Based on the hydrodynamic diameters, the peptide oligomers/aggregates were classified in 4 different families, i.e. small (0.4-5 nm), medium (20-150 nm), large (200-950 nm) and giant (1-5 □ 10³ nm). For each sample, spectra were recorded three times with 11 sub-runs using the multimodal mode. In the graphs the concentration of the oligomers belonging to different families are reported as an average±SD.

Statistical analysis: All the experiments were performed at least 3 times, except for DLS repeated 2 times. The results are presented as means±SD or SEM. The data were analyzed by GraphPad Prism (GraphPad Software, Inc., USA). * indicates P<0.05. P value was determined using one-way ANOVA with Dunnett's multiple comparison test.

### Results

### Relationship Between Turbidity and Oligomerization/Aggregation of TCP-25

TCP-25 is a 3 kDa C-terminal thrombin peptide characterized by antimicrobial and anti-inflammatory activity in vitro and in vivo. Observations that TCP-25 solutions of 300 μM TCP-25 (corresponding to 0.1 wt %) yielded a turbid appearance at pH 7.4, in contrast to pH 5 where the solution was markedly less turbid (FIG. 15A), prompted further investigations in the concentration- and pH-dependence of TCP-25 of this phenomenon. The absorbance at 405 nm and the relative transmittance of TCP-25 at pH 5-7.4 and at different concentrations was analyzed. The results, summarized in FIG. 15B, demonstrate absorbance and transmittance changes at pH 7.4 and high concentrations of TCP-25, findings compatible with the observed turbidity changes.

The possible formation of oligomers or aggregates was investigated. This was done by dissolving 300 μM TCP-25 (corresponding to 0.1 wt %) in 10 mM Tris pH 7.4 or in 10 mM NaOAc pH 5.8 or 5.0, centrifuged the samples, and analyzed both the supernatant and the pellet for TCP-25. FIG. 15C illustrates that TCP-25 indeed was detected in particular in the pellet obtained from the sample at pH 7.4. Next, to explore whether the aggregated TCP-25 was possible to redissolve, the pellet was resuspended from the pH 7.4 sample in 10 mM Tris pH 7.4 or in 10 mM NaOAc pH 5.8 or 5. The results showed the same repartition in pellet and supernatant after centrifugation as with freshly prepared TCP-25, results indicating a reversibility of the observed oligomerisation/aggregation of TCP-25. TEM was employed to visualize oligomers/aggregates. TCP-25 was dissolved in the respective pH 7.4 and 5.0 buffers at concentrations 10 μM and 300 μM and analysed by TEM. Multiple aggregates formed in the Tris-buffer at pH 7.4, particularly at 300 μM of TCP-25, which contrasted to the findings in the Acetate buffer at pH 5.0, where less aggregates where observed. The TEM images illustrating that oligomerization is pH- and concentration-dependent.

Structural Changes of TCP-25 Oligomers and their Organization

Peptide oligomerization can induce alterations in peptide secondary structure. For these analyses circular dichroism was used. TCP-25 was dissolved at different concentrations in 10 mM Tris pH 7.4 or in 10 mM NaOAc pH 5.8 or pH 5. As shown in FIG. 16A, the peptide displayed a concentration dependent increase of α-helical structure at pH 7.4, with a dominant α-helical structure recorded at the highest concentration of 300 μM TCP-25. No such marked concentration-dependent structural changes were observed at pH 5.8 or 5.0.

Given the propensity of TCP-25 to oligomerize in a concentration dependent manner at pH 7.4, which species that were formed should be further analyzed. For this purpose, freshly dissolved TCP-25 (10-300 μM) in 10 mM Tris pH 7.4 was subjected to 4-16% (w/v) Blue native (BN)-PAGE. As shown in FIG. 16B TCP-25 formed a wide range of oligomers, and parts of the material did not enter the gel, indicating large oligomers or aggregates. In order to further characterize these oligomers, TCP-25 was chemically cross-linked with BS₃. As seen in FIG. 16C TCP-25 formed a broad spectrum of oligomers in agreement with the previous results. RP-HPLC analysis on a C18 column confirmed the presence of oligomers. TCP-25 cross-linked with BS₃ at 580 μM yielded higher-order oligomers which eluted early in the gradient (FIG. 16D). The concentration dependent oligomerization was observed to be reversible. Indeed, when TCP-25 was dissolved in 10 mM Tris pH 7.4 at 1 mM concentration and then diluted to 10 or 300 μM or directly dissolved at these specific concentrations, the CD spectra for the peptide at the same concentration were perfectly overlapping.

Effects of Oligomeriztion on Tₘ and Cₘ

Denaturation midpoint of a protein is defined as the temperature (Tₘ) or concentration of denaturant (Cₘ) at which both the folded and unfolded states are equally populated at equilibrium. These parameters are changed in an oligomerized state. Thermal shift and chemical denaturation assays were employed to investigate the potential changes of Tₘ and Cₘ induced by oligomerization of TCP-25. The peptide was dissolved in 10 mM Tris pH 7.4 or in 10 mM NaOAc pH 5 at 10 and 300 μM, and subjected to thermal denaturation, by increasing the temperature from 20 to 100° C. FIG. 17A shows two representative fluorescence spectra for 300 μM TCP-25 dissolved at pH 7.4 and 5.0, respectively. In both cases the intrinsic fluorescence of the peptide decreased with increasing temperature. Same results were obtained for 10 μM TCP-25 at both pH 7.4 and 5.0. Tm was determined by fitting the maximum emission fluorescence as a function of the temperature (FIG. 17A upper panel). Tₘ was more affected by concentration than by pH changes (FIG. 17D), compatible with the observed oligomerization of TCP-25 at higher concentrations.

In order to determine the Cₘ, TCP-25 was dissolved as above followed by incubations with increasing concentrations of urea or guanidine hydrochloride (Gnd-HCl) overnight before analysis. Results obtained for TCP-25 in the presence of both chemical agents are shown in FIG. 17B-C, respectively. Analysis of the fluorescence spectra obtained for the peptide dissolved at pH 7.4 at 300 μM, showed that both urea and Gnd-HCl caused a red-shift in the maximum emission wavelength (λₘₐₓ), indicative of a change in solvent exposure of tyrosine and tryptophan residues in TCP-25 (FIG. 17B-C, upper panels). Moreover, the unfolding induced by Gdn-HCl exhibited two transition phases. The first phase of the denaturation was characterized by an increase in fluorescence intensity and a small red-shift in the λₘₐₓ in the sample with 0.5 M Gdn-HCl, indicating the formation of an intermediate which had a higher fluorescence quantum yield than TCP-25 alone in absence of the denaturing agent. Increasing the concentration of the Gdn-HCl up to 5 M, a decrease of fluorescence and a consistent red-shift, from 347 to 355 nm, in the λₘₐₓ was observed, indicating the second phase of denaturation. A completely different behavior was found for 300 μM TCP-25 dissolved at lower pH (FIG. 17B-C, upper right panels). Indeed, the λₘₐₓ of TCP-25 without any denaturant was red-shifted to 354 nm, indicated that the Trp residues were already exposed to the polar environment. Moreover, in case of denaturation by urea, a consistent increment in fluorescence intensity was also recorded, indicating that the species present in the solution were characterized by higher fluorescence quantum yield than the native form of TCP-25.

The results for 10 μM TCP-25 dissolved at pH 7.4 and 5, showed a similar denaturation profile independently of the chemical agent. In all the cases was an increase in fluorescence intensity found as well as an evident blue-shift of λₘₐₓ of TCP-25 denaturated with Gnd-HCl, an evidence of lower exposure of Trp and Tyr to the solvent. Cₘ for the two denaturants was calculated reporting the I₃₃₇/I₃₅₀ ratio as a function of the concentration of the chemical agent (FIG. 17B-C, bottom panels) and is summarized in the table in FIG. 17B. The fact that Cₘ in the presence of urea was much lower for 10 μM than for 300 μM TCP-25 indicates that observed concentration-dependent oligomerization of TCP-protects it from denaturation. For Gnd-HCl, Cₘ was possible to determine only for the 300 μM TCP-25 sample. At no denaturing condition it was possible to determine Cₘ for the peptide dissolved at pH 5. This indicated that the peptide was largely unstructured, which was in agreement with the obtained CD data. Altogether, the results from the thermal and chemical denaturation experiments are indicative of peptide aggregation/oligomerization.

### Reversibility of Thermal Denaturation of TCP-25

Thermal unfolding of a protein is generally characterized by irreversible aggregation. To investigate if this was the case also for TCP-25, the structural changes of the peptide before and after denaturation at 100° C. were analyzed. First was the intrinsic fluorescence of TCP-25 at 10 and 300 μM in 10 mM Tris at pH 7.4 or in 10 mM Acetate at pH 5, at 20° C. before and after denaturation compared. As shown on the left panel of FIG. 18A, the fluorescence of 10 μM TCP-25, dissolved at pH 7.4, increased around 1.75-fold for denatured TCP-25 when compared with the non-denatured peptide. Moreover, the λₘₐₓ was blue-shifted, indicative of aggregation of the peptide. Similar results were obtained for the peptide dissolved at pH 5 (FIG. 18B). A completely different behavior was observed in the case of TCP-25 dissolved at the same pH but at higher concentration. Indeed, the intrinsic fluorescence of TCP-25 was the same before and after exposing the peptide to 100° C., suggesting reversibility (FIG. 18A-B). Slightly higher fluorescence as well as blue-shift of λₘₐₓ was found for 300 μM TCP-25 at pH 5 after the denaturation process, compatible with a less oligomerized peptide at the initiation of the experiment (FIG. 18B).

In order to have a confirmation on the reversibility of thermal-denaturation process, the secondary structure of TCP-25 was analyzed by using CD, before and after exposing the peptide to 100° C. The peptide was dissolved at 10 and 300 μM in 10 mM Tris pH 7.4. While the conformation of the peptide was unstructured at low concentrations, similar helical spectra were obtained before and after denaturation of TCP-25 at 300 μM, demonstrating reversibility of denaturation. The data for 10 and 300 μM TCP-25 dissolved at pH 5, confirming reversibility of denaturation at higher concentrations.

### Size of Oligomers as a Function of Temperature and pH

To get further insight into the size of the oligomers and their relative distributions, dynamic light scattering (DLS) was employed. TCP-25 was dissolved in 10 mM Tris pH 7.4 or in 10 mM NaOAc pH 5 at 300 μM immediately before the first measurement. FIG. 19A shows the results for the peptide at both pH values. The Z-average (mean particle size) was found to be higher for TCP-25 dissolved at pH 7.4 than at pH 5, indicating that the peptide forms bigger oligomers at pH 7.4. Interestingly, the polydispersity index (Pdi) was 0.68 in both cases, suggesting the presence of different species in the solution (Pdi<0.1 means that the sample is monodispersed).

In the next step the temperature dependence of the oligomerization was investigated. Results depicted in FIG. 19B were obtained after analysis of TCP-25 stored for 24 h at RT, 4 and −20° C. A much higher decay time as well as intensity were observed for the peptide at pH 7.4, at all storage conditions, with respect to the peptide at pH 5, which indicate an increase in TCP-25 size. At pH 5 was only moderate rise of intensity with the temperature decrease found, since low temperature generally promotes hydrophobic interactions. FIG. 19C-E shows the size distribution of the oligomers and their concentrations in solution. For the freshly prepared sample (indicated as time 0 in the graphs) the particles were spanning a broad range of hydrodynamic diameters detected. Therefore, they were classified in 3 families: small (0.4-5 nm), medium (20-150 nm) and large (200-950 nm). Notably, the same species were found independently of the pH of the buffer at which the peptide was resuspended. At pH 7.4 a moderately higher number of medium-sized oligomers were detected, and after 1 h at RT, the TCP-25 solution was slightly hazy. Indeed, the results showed that large species with hydrodynamic diameters spanning 1*10³-5*10³ nm were identified. Of note was also that similar results were obtained when the sample was analyzed over time (up to 1 week). TCP-25 stored at pH 5 was completely limpid, and the oligomers detected at pH 5 after 1 h and up to 1 week were identical to freshly dissolved TCP-25. The fact that similar results were obtained when both samples were analyzed over time (from 1 h up to 1 week), indicates the rapid formation of stable equilibria at the respective pH used.

Analysis of the size distribution storage at 4 or −20° C. was also performed. Whereas the results from pH 5.0 showed a similar size distribution, the pH 7.4 sample contained large oligomers and aggregates of even larger size (FIG. 19C, upper right panel). However, the largest population disappeared after 15 minutes, and a large number of medium diameter oligomers appeared in their place. The same results was obtained after storing the samples at RT for 75 min. Taken together, the results summarize the pH dependence of TCP-25 oligomer formation and the influence of storage conditions.

### Discussion

Defining the oligomerisation behaviour of TCP-25 and its prerequisites provides an explanation for the observed turbidity of the formulated TCP-25 hydrogel.

The organization of peptides in oligomers or aggregates is often associated with induction of toxicity and immunogenicity as well as with a loss in their activity. However, for other groups of peptides oligomerization or aggregation is an intrinsic part of the peptide's natural mode of action. Oligomerisation and aggregation can therefore be compatible with peptide functionality. TCP-25 was found to oligomerize in a reversible manner, compatible with its observed efficacy in multiple in vitro and in vivo models.

The sequence of TCP-25 contains a pH-responsive histidine residue, which is protonated at low pH rendering the peptide more charged, with a change in net charge from +2 to +3 at low pH. This may lead to alterations in its amphipathic region and increase in peptide solubility, leading to reduced oligomerization. These results are reinforced by data showing that charged histidine has a low helix propensity. Protonation at pH 5.5 of this particular histidine residue also increases the antibacterial activity of TCP-25 against Gram-negative Escherichia coli by membrane disruption. Moreover, TCP-25 display a decreased binding affinity to human CD14 with decreasing pH, suggesting a switch in mode-of-action, from more anti-inflammatory at neutral pH to more antibacterial at acidic pH. It is demonstrated that a subtle protonation of the histidine residue in TCP-25 affects not only activity but also peptide conformation and oligomerisation tendency.

CD analysis combined with DLS showed that a conformational change in TCP-25 accompanies peptide association and oligomerization. From a therapy perspective, an improved understanding of the oligomerization prerequisites and consequences can facilitate the preclinical and regulatory development of TCP-25. It was demonstrated that oligomeric TCP peptides are more stable and have higher Tₘ and Cₘ with respect to the monomeric peptides. Analogously TCP-25 was more resistant to both chemical and thermal denaturation under conditions that favored oligomerization. In particular, the thermal stability is very important, since therapeutic peptides have to withstand a number of processes during production, such as filtration and sterilization, and be subjected to a long storage before they can be placed on the market. Accordingly, oligomerization could be exploited as a stabilizer of TCP-25, since the surface area will be smaller than in the monomer, and hence, the peptide will be less prone to denaturation and protease cleavage. It is also possible that oligomerization could facilitate a slower release of active molecules. Indeed, the active monomers were gradually released from the oligomers, but this release was dependent on sequence and pH at which oligomers were assembled.

The size range of the TCP-25 oligomers was broad, but some sizes were more recurrent than the others, such as oligomers with the hydrodynamic diameters of 0.46, 2.81, 4.58, 43, 230, 431, 462, 808 and 1740 nm. Furthermore, the observed continuous change in the sizes of the particles in solution indicates that oligomerization of TCP-25 is a dynamic process that reaches an equilibrium after different lengths of time and depending on the conditions. This flexibility of the peptide to assume different conformations and form oligomers of different sizes may contribute not only to stability, but also to activity and specificity, as reported for other proteins as well as AMPs.

In conclusion, it is demonstrated that TCP-25 has an increase in α-helical structure as well as oligomerisation at higher doses at neutral pH. TCP-25 is also more stable at higher concentrations when exposed to high temperatures or denaturing agents, which is compatible with oligomer formation.

### Example 8

### Boosting the Antibacterial Effects of the Thrombin-Derived Peptide TCP-25

EDTA is a metal chelating agent that is known to exert antioxidant effect. Interestingly, the results described in this example showed that the combination of TCP-25 with EDTA at physiological pH led to an immediate oligomer formation, yielding a turbid appearance. Unexpectedly, it was found that this precipitation was largely abolished at pH 5.0. This was advantageous, as TCP-25 has been shown to exert an increased bacterial membrane permeabilization at acidic pH. While EDTA alone showed some bacteriostatic effect, the combination of TCP-25 and EDTA led to a significant boosting of TCP-25 effects on planktonic and biofilm-associated bacteria in vitro when combined with EDTA, particularly at pH 5. Moreover, unexpectedly, storage stability was significantly improved at low pH by EDTA. Compared with the TCP-25 hydrogels without EDTA, the TCP-25+EDTA pH 5.0 hydrogel described in this example showed improved efficacy in relevant ex vivo skin wound models. Thus, the TCP-25+EDTA pH 5.0 hydrogel counteracted the common wound pathogens S. aureus and P. aeruginosa in biofilms and ex vivo wound infection models.

### Methods

MIC, MBC, and time kill assays were employed in order study the effects of TCP-25 in combination with EDTA at different pH conditions on planktonic bacterial cells and biofilms. Live/dead assay followed by microscopy analysis was used to visualise and quantify antimicrobial effects. An ex vivo porcine skin wound infection model was used to translate the obtained results to physiologically relevant conditions. Stability was analysed by HPLC.

### Peptides, Buffers, and Gel Formulations

The thrombin-derived peptide TCP-25 (GKYGFYTHVFRLKKWIQKVIDQFGE)(SEQ ID NO:1) (97% purity, Acetate salt) was synthetized by Ambiopharm (Madrid, Spain). If nothing else is indicated, the term TCP-25 refers TCP-25 of SEQ ID NO:1. For this study we used two buffer systems, Tris at pH 7.4 and Acetate at pH 5.0 buffer, both at a concentration of 10 or 25 mM. For each respective buffer an additional stock containing 40 mM of EDTA, di sodium salt dihydrate (Sigma Aldrich, Saint Louis, Mo., USA) was prepared. The gel-forming substance used in this study was hydroxyethyl cellulose (HEC, Natrosol™ 250 HX, MW 1000000; Ashland Industries Europe GmbH, Schaffhausen, Switzerland). For achieving isotonicity of the formulation, glycerol was added to the buffers, 2% in the 10 mM buffers and 1.9% in the 25 mM buffers. EDTA was added to the formulations, yielding final concentrations of 1, 2.5, 5, and 10 mM, respectively. The gel was prepared by preheating buffer to 56° C. for 30 minutes prior to addition of the HEC powder (1.5% w/v). A magnetic stirrer was used to form homogenous gels, which were then centrifuged for 5 min at (3.5×1000 rpm) (to remove air bubbles), directly after mixing. Gels were left for and additional 5 min at room temperature prior to adding the peptide solution at final concentrations of 0.1, 0.5 or 1%, previously resuspended in a small volume of respective buffer. These concentrations correspond to 0.3 mM, 1.5 mM and 3 mM, respectively. Homogeneity of the peptide was ensured using firstly magnetic stirrer, followed by vigorous shaking. Tubes were again centrifuged for 5 min.

### Bacteria and Growth Conditions

Todd Hewitt (TH) broth was solidified by adding 15 g/l of BactoAgar, in 37° C. overnight and then kept in 4° C. until cultivation. One colony of bacteria was inoculated in 5 ml of TH broth and incubated at 37° C. overnight. Bacterial culture was refreshed in 5 ml TH-broth the following day and grown to mid-logarithmic phase (OD₆₀₀=0.4). After washing, the bacterial pellet was diluted to make a 1% bacterial solution (1-2×10¹ CFU/ml). The bacterial strains used for this study includes Staphylococcus aureus, ATCC 29213 and clinical isolates 1781, 1779, 2278, 2279, 2788, 2404, 2528, 2789, Pseudomonas aeruginosa PAO1 and clinical isolates 51:1, 25:1, 10:5, 23:1, 62:2, 15159, 18488, and Escherichia coli, ATCC 25922.

### Minimal Inhibitory Concentration (MIC) and Minimal Bactericidal Concentration (MBC)

96-well round bottom polystyrene plates (Corning INC, Kennebunk, USA) were used to assess the antimicrobial effects of TCP-25 in combination with EDTA. The minimal inhibitory concentration (MIC) was conducted according to standard protocol (Wiegand et al., 2008). A 1% bacterial solution was diluted 1:1000 times in 2×BBL™ Mueller Hinton (MH) II, cation adjusted broth (Becton, Dickinson and Company, Sparks, USA). Wells were prepared with 50 μl MH broth with serial diluted treatment conditions, ranging from 1.25-160 μM of TCP-25, and EDTA 0-10 mM. Next, 50 μl of the bacterial solution was added. MH broth without bacteria was used as a sterile control, whereas supplemented only with bacteria as growth control. Plates were incubated at 37° C. for 24 hours prior to MIC analysis. MIC was assessed as the lowest concentration of treatment that prevents visual bacterial growth in the wells. The MIC plates were then used to determine the minimal bactericidal concentration (MBC) for the various treatments. This was conducted by resuspending the solution in each well with a pipette and then plating 10 μl droplets on a THA plates which were then incubated in 37° C. overnight. MBC was established at concentration at which no bacterial colonies were observed.

### Viable Count Assay (VCA) and LIVE/DEAD™ Staining

10 μl of 1% bacterial solution from either ATCC 29213 or PAO1 was added to 40 μl of 10 mM Tris at pH 7.4 or 10 mM Acetate buffer at pH 5.0 with or without 2.5 mM EDTA. TCP-25 of SEQ ID NO:1 was thereafter added to reach the final concentrations of 80 μM. Bacteria supplemented only with buffer were used as a control. Following 60 minutes incubation at 37° C., the samples were vortexed and 10 μl of each sample were serially diluted in PBS and plated on THA plates. CFU were determined by counting bacterial colonies after overnight incubation at 37° C. For microscopic visualisation of bacterial killing, a LIVE/DEAD assay was conducted. Briefly, bacteria were treated as for VCA. After 1 hour incubation, 50 μl of bacteria were diluted 1:1 in LIVE/DEAD™ solution. 1 ml of LIVE/DEAD™ solution was prepared by adding 1.5 μl of each component (component A, SYTO® 9 green-fluorescent nucleic acid stain and component B, red-fluorescent nucleic acid stain propidium iodine) from the BacLight™ Bacterial Viability Kit L-7012, into 995 μl of PBS. Samples were incubated under dark for 15 min prior to centrifugation a 14000 rpm for 5 minutes. 80 μl were discarded from the tubes and the pellet was resuspended in the remaining solute. A droplet (5 μl) was placed on Superfrost® Plus microscopic slides (Thermo Fisher) and analysed by fluorescent microscopy.

### Time-Kill Assay and LIVE/DEAD™ Staining

Using the same treatment conditions as for the VCA assay, a time-kill assay was conducted. A 1% bacterial solution of ATCC 29213 or PAO1 was further diluted, 1:1000 in 2×MH broth. 500 μl of bacterial solution was supplemented with 500 μl of treatment in 10 ml culture tubes and placed on a shaker at 180 rpm at 37° C. Samples were collected continuously after 5, 10, 15, and 30 min and 1, 3, 6 and 24 hours. Samples from each time point were serially diluted and plated on THA plates. Bacterial colony units were counted the following day to determine CFU/ml. The LIVE/DEAD staining was then used to visualize bacterial killing, as described above.

### Biofilm Studies

The biofilm of ATCC 29213, was grown on Costar™ 96-well, round bottomed vinyl flexible plates (Coring Incorporated, Kennebunk, USA) in 0.5% Tryptic soy broth (TBS) supplemented with 0.2% glucose. PAO1 biofilm was grown in M63 medium supplemented with 0.5% casamino acids, 0.2% glucose and 1 mM MgSO₄, on flat bottom 96-well microplates (Greiner Bio-One, Frickenhausen, Germany). Respective growth media (100 μl) were supplemented with 5 μl bacterial solution at 1×10⁸ CFU/ml. Next, plates were covered with microplate seals and placed in moist containers and incubated at 37° C. for 48 hours to ensure mature biofilms. Before treatment, the planktonic cells were removed from the biofilm by washing it with 100 μl of PBS. Then, 100 μl of different treatment was added to the wells. The treatment used were: 1) Buffer 2) Buffer+2.5 mM EDTA 3) Buffer+0.1% TCP-25 and 4) Buffer+2.5 mM EDTA+0.1% TCP-25. Four buffer conditions were used for this study, i.e. 10 or 25 mM of Tris at pH 7.4 and 10 or 25 mM Acetate at pH 5. The treatment was then added to the biofilm as either a gel or as a solution. After addition of treatments the plates were again sealed and incubated in 37° C. for an additional 2 hour. For the assays where the treatment was added as a solution, the treatment and planktonic cells were removed and discarded. Biofilms were then washed twice in 100 μl of PBS. 200 μl of PBS was then added to the wells and the biofilm was disrupted through scratching using a pipette tip. 10 μl was then sampled from each well, serially diluted and plated for CFU determination. To prevent bacterial killing during the extraction phase, 100 μl of 10 mg/ml dextran sulphate in PBS was added prior to disruption of the biofilm. Dextran sulphate neutralizes the antibacterial effect of TCP-25. μl sample was serial diluted and plated on THA plates. Bacterial colonies were counted the following day for CFU determination.

### Ex-Vivo Pig Skin Burn Wound Model

Porcine skin grafts from Gottingen minipigs were frozen at −20° C. until further use. The skin was defrosted for 2 hours on petri dishes and then washed with 96% ethanol prior to use. Wounding was created according to the method described by Andersson et al (2020). In short, a soldering iron with 08 mm, was held against the graft for 15 seconds to create a burn wound.

Two wounds per treatment were made on each graft. After burning, PBS was added to the dish, keeping the tissue moist during incubations. A 1% bacterial solution of E. coli ATCC 29213 or P. aeruginosa PAO1 was diluted 1:10 in 10 mM Tris buffer at pH 7.4, and 30 μl was then added to each wound. Parafilm was placed over the graft, further preventing evaporation, and the plates were then placed into the 37° C. incubator for 2 hours, allowing for infection. 100 μl of treatments containing various concentrations of TCP-25 of SEQ ID NO:1 (0.1-1%) and EDTA (5-20 mM), diluted in either Tris or Acetate buffer were then added to the wounds. The skin graft was covered with a fresh piece of parafilm and placed back into the incubator for another 2 hours. Material from the surface and the tissues were then analysed. For the topical sampling, the wounds were washed twice in 40 μl neutralizing agent (10 mg/ml of dextran sulphate in PBS buffer). Washings were collected, serially diluted and plated on THA plates. Tissue samples were obtained from homogenized tissues. For this the wounds were cut from the graft using a scalpel, and then cut into smaller pieces before being placed in a 2 ml SC Micro Tube PCR-PT tubes (Sarstedt, Numbrecht, Germany) together with 500 μl of neutralizing agent and approximately 30 ceramic beads (1.4 mm) (Qiagen Gmbh, Hilden, Germany).

Homogenizing was conducted using a Roche MagNA Lyser, set to 6000 rpm for 30 s repeated 4 times, with 1 minute between runs. Then the serial dilutions of all samples were plated on THA plates. The following day, colonies were counted and CFU were determined. A schematic representation of infection ex vivo model is presented in FIG. 6A.

### High Pressure Liquid Chromatography (HPLC)

The effect of pH on the stability of TCP-25 was analyzed by reverse-phase C18 chromatography on a Phenomenex Kinetex C18-column (150×4.6 mm 2.6 μM, 100 Å pore size, California, USA) by using the Agilent 1260 Infinity System. The column was equilibrated using 95% of buffer A containing 0.25% of Trifluoroacetic Acid (TFA) in MilliQ and 5% of Buffer B containing 0.25% of TFA in acetonitrile. The peptide was dissolved in OmniPur WFI Quality Water (EMD Millipore Corporation, Billerica, Mass., USA) and the pH was corrected to 5, 6 or 7.4 with HCl or NaOH accordingly. Then the volume was adjusted to reach the final concentration of the pure peptide in solution equal to 0.1%. The samples were then stored at RT, 4, 37 or 70° C. Immediately before injection the peptide was dissolved in Buffer A (1:7), and 5 μg were injected onto the system. The elution profile was monitored during the gradient (35% of B at 10 min, 45% at 20 min) and the spectrum at 215 nm was recorded. The flow rate of the column was 1 mL/min and all runs were performed at 50° C. The data are presented as the percentage of total area that corresponds to the sum of the area of all eluted peaks (100%).

To test the stability of TCP-25 in the presence of EDTA, the peptide was dissolved at 0.1% in 25 mM Acetate buffer with or without 10 mM EDTA and stored at RT, 4 or 37° C. before analysis by reverse-phase C18 chromatography as reported above. For testing stability at different pHs, the peptide was dissolved at 0.1% in distilled water and the pH was then corrected by adding NaOH or HCl to reach the indicated pH.

### Statistics

All microbiological and microscopic assays demonstrated in this study is represented by at least 3 replicate experiments. Data is presented as mean±SEM. Statistical analysis were performed using GraphPad Prism software version 8. Significant differences between conditions were determined using One-way ANOVAS or repeated measurements Two-way ANOVAS with Tukey's post hoc tests for multivariate analysis.

### Results

TCP-25 formed large oligomers with EDTA at pH 7.4, a phenomenon not observed at pH 5.0. The combination of TCP-25, pH 5.0 buffer, and EDTA dramatically lowered both MIC and MBC, as well as prevented regrowth of bacteria over a 24-hour time period. The hydrogel formulation comprising both TCP-25 and EDTA described herein was shown to be highly effective against both S. aureus and P. aeruginosa bacteria in mature biofilms in vitro. Moreover, in an infected pig skin wound model, the formulation was shown to be significantly more effective in reducing bacteria at the wound surface as well as in the underlying tissue. EDTA improved storage stability at pH 5.0.

### EDTA Effects on TCP-25 in Various Buffer Conditions

Addition of 5-10 mM EDTA to TCP-25 of SEQ ID NO:1 in Tris buffer at pH 7.4 yielded visible turbidity of the solution. Similar turbidity became evident also after addition of 0.5-2.5 mM EDTA, but only after incubation of the sample at room temperature for at least 30 min. After centrifugation a visible white pellet was formed. The particles were possible to solubilise in buffer which demonstrated a reversibility of the process, suggesting that observed white hazy deposit was oligomer formation of the peptide. When TCP-25 was mixed in a HEC based hydrogel in Tris at pH 7.4, a visible change it the turbidity of the gel was observed (FIG. 20A). This turbidity was even more pronounced in the presence of 2.5 mM EDTA. Interestingly, a less turbid formulation was observed when Acetate buffer at pH 5 was used (FIG. 20A). These data are in agreement with the results on oligomerization of TCP-25 at higher pH described in Example 7.

### Effect of EDTA on Antimicrobial Activity of TCP-25

EDTA reduced MIC and MBC for TCP-25 of SEQ ID NO:1. This reduction was more evident when the peptide was diluted in pH 5 compared to pH 7.4. Using the Acetate buffer (pH 5), a reduction in MIC could be observed at the lowest concentration of added EDTA (0.5 mM), however using Tris, 1-2.5 mM of EDTA was required to initiate a boost in antibacterial effects. At 2.5 mM EDTA in either Tris or Acetate, MIC for TCP-25 of SEQ ID NO:1 had decreased from 20-40 μM to 1.25 μM for all three bacterial strains (FIG. 20B). MBC was also greatly reduced as a function of EDTA addition. As demonstrated for MIC this effect was most pronounced at pH 5 buffer, reducing MBC for TCP-25 (SEQ ID NO:1) from 160 μM to 1.25 μM. For pH 7.4 none of the EDTA concentrations used in this study reduced MBC for TCP-25 of SEQ ID NO:1 to the lowest peptide concentration but still demonstrated a reduction from 160 μM to 5-10 μM TCP-25 of SEQ ID NO:1 (FIG. 20C). To further validate these effects, clinical isolates of S. aureus and P. aeruginosa were assessed and a similar effect of EDTA boosting was observed (Table 7). Similarly, the MBC was also reduced for the clinical isolates and this effect was more pronounced when using Acetate buffer (Table 8).

TABLE 7
MIC and MBC values for clinical isolates of S. aureus and P. aeruginosa. TCP-25 (SEQ ID NO: 1) and
EDTA concentrations are indicated. Experiments were performed in 10 mM Tris buffer, pH 7.4 (n = 3).

| | EDTA mM | | | | | | | | | | | |
|---|---|---|---|---|---|---|---|---|---|---|---|---|
| | 10 | | 5 | | 2.5 | | 1 | | 0.5 | | 0 | |
| | MIC | MBC | MIC | MBC | MIC | MBC | MIC | MBC | MIC | MBC | MIC | MBC |
| S. aureus | | | | | | | | | | | | |
| 1781 | ≤1.25 | ≤1.25 | ≤1.25 | ≤1.25 | ≤1.25 | 10 | 2.5 | 10 | 20 | 40 | 40 | 40 |
| 1779 | ≤1.25 | 2.5 | ≤1.25 | 2.5 | ≤1.25 | 2.5 | ≤1.25 | 2.5 | 20 | 10 | 20 | 40 |
| 2278 | ≤1.25 | 2.5 | ≤1.25 | 40 | ≤1.25 | 40 | ≤1.25 | 40 | 10 | ≥160 | 20 | ≥160 |
| 2279 | ≤1.25 | ≤1.25 | ≤1.25 | ≤1.25 | ≤1.25 | ≤1.25 | ≤1.25 | ≤1.25 | 20 | 2.5 | 20 | 2.5 |
| 2788 | ≤1.25 | 10 | ≤1.25 | 40 | ≤1.25 | 40 | ≤1.25 | 160 | 10 | 160 | 20 | 160 |
| 2404 | ≤1.25 | 40 | ≤1.25 | 40 | ≤1.25 | 40 | ≤1.25 | 40 | 20 | 40 | 40 | ≥160 |
| 2528 | ≤1.25 | 10 | ≤1.25 | 10 | ≤1.25 | 40 | ≤1.25 | 40 | 20 | ≥160 | 40 | ≥160 |
| 2789 | ≤1.25 | 10 | ≤1.25 | 10 | ≤1.25 | 10 | ≤1.25 | 40 | 5 | 160 | 10 | ≥160 |
| P. aeruginosa | | | | | | | | | | | | |
| 51:1 | ≤1.25 | ≤1.25 | ≤1.25 | ≤1.25 | ≤1.25 | 10 | 5 | 40 | 10 | 10 | 80 | ≥160 |
| 25:1 | ≤1.25 | ≤1.25 | ≤1.25 | ≤1.25 | ≤1.25 | ≤1.25 | 10 | ≤1.25 | 40 | ≥160 | 80 | ≥160 |
| 10:5 | ≤1.25 | ≤1.25 | ≤1.25 | ≤1.25 | ≤1.25 | ≤1.25 | ≤1.25 | ≤1.25 | 40 | 160 | 80 | ≥160 |
| 23:1 | ≤1.25 | ≤1.25 | ≤1.25 | ≤1.25 | ≤1.25 | ≤1.25 | ≤1.25 | 2.5 | 80 | 80 | 80 | 80 |
| 62:2 | ≤1.25 | ≤1.25 | ≤1.25 | ≤1.25 | ≤1.25 | 5 | ≤1.25 | 5 | 40 | 80 | 80 | 80 |
| 15159 | ≤1.25 | ≤1.25 | ≤1.25 | 20 | ≤1.25 | 20 | ≤1.25 | 40 | 80 | 80 | 160 | ≥160 |
| 18488 | ≤1.25 | ≤1.25 | ≤1.25 | ≤1.25 | ≤1.25 | 5 | ≤1.25 | 20 | 160 | 20 | ≥160 | ≥160 |

TABLE 8
MIC and MBC values for clinical isolates of S. aureus and P. aeruginosa. TCP-25 (SEQ ID NO: 1) and
EDTA concentrations are indicated. Experiments were performed in 10 mM Acetate buffer, pH 5 (n = 3).

| | EDTA mM | | | | | | | | | | | |
|---|---|---|---|---|---|---|---|---|---|---|---|---|
| | 10 | | 5 | | 2.5 | | 1 | | 0.5 | | 0 | |
| | MIC | MBC | MIC | MBC | MIC | MBC | MIC | MBC | MIC | MBC | MIC | MBC |
| S. aureus | | | | | | | | | | | | |
| 1781 | ≤1.25 | 40 | ≤1.25 | 40 | ≤1.25 | 40 | ≤1.25 | ≥160 | 20 | ≥160 | 20 | ≥160 |
| 1779 | ≤1.25 | ≤1.25 | ≤1.25 | ≤1.25 | ≤1.25 | ≤1.25 | ≤1.25 | 5 | 2.5 | 20 | 5 | ≥160 |
| 2278 | ≤1.25 | 2.5 | ≤1.25 | 2.5 | ≤1.25 | 2.5 | ≤1.25 | 2.5 | 5 | 40 | 20 | 40 |
| 2279 | ≤1.25 | 2.5 | ≤1.25 | 2.5 | ≤1.25 | 10 | ≤1.25 | 10 | 2.5 | 20 | 20 | 40 |
| 2788 | ≤1.25 | 160 | ≤1.25 | 160 | ≤1.25 | 160 | ≤1.25 | 160 | ≤1.25 | ≥160 | 20 | ≥160 |
| 2404 | ≤1.25 | 40 | ≤1.25 | 40 | ≤1.25 | 160 | ≤1.25 | 160 | ≤1.25 | 160 | 10 | 160 |
| 2528 | ≤1.25 | 10 | ≤1.25 | 40 | ≤1.25 | 40 | ≤1.25 | 40 | ≤1.25 | 160 | 10 | 160 |
| 2789 | ≤1.25 | 10 | ≤1.25 | 10 | ≤1.25 | 10 | ≤1.25 | 40 | ≤1.25 | 160 | 10 | ≥160 |
| P. aeruginosa | | | | | | | | | | | | |
| 51:1 | ≤1.25 | 10 | ≤1.25 | 1.0 | ≤1.25 | 1.0 | 40 | 40 | ≥160 | ≥160 | 40 | ≥160 |
| 25:1 | ≤1.25 | 10 | ≤1.25 | 1.0 | ≤1.25 | 1.0 | 40 | ≥160 | ≥160 | ≥160 | 40 | ≥160 |
| 10:5 | ≤1.25 | 10 | ≤1.25 | 1.0 | ≤1.25 | 1.0 | 40 | 40 | 80 | ≥160 | 40 | ≥160 |
| 23:1 | ≤1.25 | ≤1.25 | ≤1.25 | 2.5 | ≤1.25 | 1.0 | ≤1.25 | 10 | 20 | 40 | 20 | 40 |
| 62:2 | ≤1.25 | ≤1.25 | ≤1.25 | ≤1.25 | ≤1.25 | ≤1.25 | ≤1.25 | 5 | ≤1.25 | 5 | 20 | 40 |
| 15159 | ≤1.25 | 2.5 | ≤1.25 | 2.5 | ≤1.25 | 40 | ≤1.25 | 160 | 20 | 160 | 20 | 160 |
| 18488 | ≤1.25 | ≤1.25 | ≤1.25 | ≤1.25 | ≤1.25 | ≤1.25 | ≤1.25 | 2.5 | 2.5 | 2.5 | 20 | ≥160 |

### TCP-25 Induces Bacterial Killing in the VCA Assay

Using a VCA assay it was found that the bacterial cells were significantly reduced when treated with TCP-25 of SEQ ID NO:1 and EDTA. Interestingly, there was a significant difference in cell reduction as an effect of the buffer used (two-way ANOVA; p<0.0001), where Acetate had a higher killing of bacteria, showing a 99.9-100% cell reduction when treated with TCP-25 (SEQ ID NO:1)±EDTA (p<0.001) (FIG. 21). Visualization of the bacterial reduction was done using microscopic images using the LIVE/DEAD assay. In the treatments containing TCP-25 of SEQ ID NO: 1 bacterial aggregates were observed. Information regarding aggregate size distribution is presented in FIG. 22.

### EDTA Boosts the Anti-Microbial Effects of TCP-25

To explore the effects of TCP-25 and EDTA over time we conducted a time kill assay. We found that for this assay there were no significant differences in antibacterial properties that were related to the buffer system used. TCP-25 of SEQ ID NO:1 has a fast-acting antibacterial effect independently of EDTA being present or not. For S. aureus we observed a 100% reduction of CFU after 5 min when treated with TCP-25±EDTA. Interestingly, samples treated with TCP-25 alone are recolonized at 6 and 24 hours, however for the combined TCP-25+EDTA treatment a 100% cell reduction was observed throughout the duration of this experiment (FIG. 23A). To visualize antibacterial effects of the various treatments, fluorescent microscopy using LIVE/DEAD staining was conducted, confirming the results presented for CFU. Bacterial clusters, aggregates, were observed during microscopy in the various treatments containing TCP-25 and/or EDTA in both 1- and 24-hours samples (FIG. 23B). Various sizes of bacterial aggregates were found with larger sized aggregates are present in a higher degree at the 24 hours measuring point. Aggregate formation was more evident when using Acetate buffer. Furthermore, in Acetate, TCP-25 of SEQ ID NO:1 does not cause aggregation when EDTA is not present at the 1-hour time point, but over time TCP-25 (SEQ ID NO alone cause the bacteria to aggregate.

TCP-25 causes a significant reduction in P. aeruginosa, but after 24 hours, the bacteria had recolonized. However, similar as with S. aureus, a 100% reduction in bacterial CFU was apparent throughout the experiment in treatment with TCP-25 (SEQ ID NO:1)+EDTA (FIG. 24A). These results were further visualized by fluorescent microscopy. Bacterial aggregation was found to be present also in P. aeruginosa treated with TCP-25 (SEQ ID NO:1) and TCP-25+EDTA. In Tris, we see a higher proportion of large aggregates at 1 hour when treated with TCP-25 (SEQ ID NO:1) alone and over time these aggregates are reduced, and a larger proportion of smaller aggregates becomes apparent. This shift of aggregate size apparent at the 24 hour time point is not present when using Acetate buffer (FIG. 24B).

### Effects of EDTA on TCP-25's Ability to Reduce Biofilm Associated Bacteria.

Treatment of mature biofilms from S. aureus and P. aeruginosa, demonstrated that independently of the buffer used (10 mM Tris or 10 mM Acetate), TCP-25 (SEQ ID NO:1) and TCP-25 (SEQ ID NO:1)+EDTA reduced bacterial cells within the biofilm. When complementing with 2.5 mM EDTA to the peptide solution, we found a 99% reduction in bacterial cells, for both bacteria used (FIGS. 25A and B). Similar results were demonstrated for TCP-25 and EDTA combinations using 25 mM Tris and Acetate buffers (FIGS. 25C and D).

### Effects of TCP-25 and EDTA in a Porcine Skin Wound Infection Model Skin

Treatment containing various doses of TCP-25 of SEQ ID NO:1, showed a dose dependent reduction in CFU, compared to the control, when analysing treatment effect topically on wounds treated with P. aeruginosa. Where 1% TCP-25 had a significantly higher reduction in CFU, compared to 0.1% of TCP-25 (p<0.05). The analyses demonstrated a significant reduction of bacterial cells also within the tissue when compared to the control, however, and all three treatments was shown to have a 90% reduction of bacteria inside the tissue of the wound (FIG. 26A). Addition of EDTA significantly boosted the effects of TCP-25 on P. aeruginosa, demonstrating a dose dependent decrease in CFU associated with an increasing concentration of EDTA in combination with 0.1% TCP-25, where 20 mM of EDTA induced a 6-log reduction in CFU. 20 mM EDTA+0.1% TCP-25 demonstrated a significant reduction of bacterial cells in the tissue, reducing bacteria with approximately 90% (FIG. 26B). Next, we proceeded by testing 10 mM EDTA with various concentrations of TCP-25, 0.1, 0.5 or 1% on both P. aeruginosa and S. aureus infected wounds. Significant effects of the treatments were found topically for both bacterial strains, demonstrating a 6-log reduction for P. aeruginosa and a 3-log reduction for S. aureus when treating with the highest concentration of TCP-25 of SEQ ID NO:1. A significant reduction of bacteria was detected for P. aeruginosa, with reductions by 90% (FIG. 26C).

### Effects of EDTA and pH on TCP-25 Stability

As illustrated in FIG. 27, the stability of TCP-25 of SEQ ID NO:1 in Acetate buffer with or without EDTA was analysed. The peptide was dissolved at 0.1% in Acetate buffer with or without EDTA and stored at RT, 4 or 37° C. before analysis. The results showed that EDTA protected the peptide from degradation. Next, stability of TCP-25 of SEQ ID NO:1 was analysed at different pHs. The peptide was dissolved at 0.1% in distilled water then the pH was corrected by adding NaOH or HCl to reach the desired pH. The samples were then stored at RT, 4, 37 or 70° C. before HPLC analysis. TCP-25 showed an increased degradation at pH 5.0. At pH 7.4, TCP-25 oligomerises at or above 0.1%, leading to a significant reduction of peptide degradation (see FIG. 28).

### Discussion

In this example, a boosting effect of EDTA on the antibacterial and antibiofilm properties of TCP-25 is shown. Furthermore, it is shown that the TCP-25 EDTA combination is optimised with respect to solubility and performance particularly at pH 5.0. When studying the time dependent effects of TCP-25, the peptide significantly reduced bacterial levels. However, in absence of EDTA there was a noticeable regrowth of bacteria after 24 h. Importantly, addition of EDTA yielded a significant and prolonged antibacterial effect of TCP-25 during the 24 hour incubation time. TCP-25 alone was demonstrated to penetrate mature biofilms and to reduce biofilm associated S. aureus and P. aeruginosa bacteria. Addition of EDTA boosted these effects, and a 99% reduction of bacteria was demonstrated in the mature biofilms. This is of relevance as the model may better represent the in vivo situation, where possible interference from substances such as metals or other EDTA scavengers may occur. Nevertheless, it was notable that the TCP-25 10 mM EDTA combination was able to significantly reduce the bacterial levels at the wound surface as well as inside the tissue. Importantly, EDTA significantly improved stability of TCP-25 at low pH.

### REFERENCES

K. Lundqvist, H. Herwald, A. Sonesson, A. Schmidtchen, Heparin binding protein is increased in chronic leg ulcer fluid and released from granulocytes by secreted products of Pseudomonas aeruginosa. Thromb Haemost 92, 281-287 (2004).
I. Wiegand, K. Hilpert, R. E. Hancock, Agar and broth dilution methods to determine the minimal inhibitory concentration (MIC) of antimicrobial substances. Nat Protoc 3, 163-175 (2008).
J. D. Morrisett, J. S. David, H. J. Pownall, A. M. Gotto, Jr., Interaction of an apolipoprotein (apoLP-alanine) with phosphatidylcholine, Biochemistry, 12 (1973) 1290-1299.

1-25. (canceled)

26. A composition comprising:
a) a compound comprising a peptide comprising or consisting of the amino acid sequence
X₁-X₂-X₃-X₄-X₅-X₆-W-X₈-X₉-X₁₀, wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues, and
wherein the concentration of the compound in the composition is at least 0.08 wt %, for example in the range of 0.08 to 3 wt %.

27. A composition comprising:
a) a compound comprising a peptide comprising or consisting of the amino acid sequence
X₁-X₂-X₃-X₄-X₅-X₆-W-X₈-X₉-X₁₀, wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues, and
b) EDTA and
c) an aqueous buffer,
wherein
i. the composition has a pH of at the most 8 and/or
ii. the concentration of the compound in the composition is at least 0.08 wt %.

28. A composition comprising:
a) a compound comprising a peptide comprising or consisting of the amino acid sequence
X₁-X₂-X₃-X₄-X₅-X₆-W-X₈-X₉-X₁₀, wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues,
b) a non-ionic polymer capable of forming a hydrogel when mixed with an aqueous solution, and
c) an aqueous solution
wherein
i. the concentration of the compound in the composition is at least 0.08 wt % and/or
ii. the non-ionic polymer is present in said composition at a concentration of at least 0.05 wt %.

29. The composition according to claim 26, wherein the composition further comprises EDTA.

30. The composition according to claim 26, wherein the composition is a hydrogel or a viscous solution, preferably the composition is a hydrogel.

31. The composition according to claim 26, wherein the non-ionic polymer is selected from the group consisting of polyallylalcohol, polyvinylalcohol, polyacrylamide, polyethylene glycol (PEG), polyvinyl pyrrolidone, starches, such as corn starch and hydroxypropylstarch, alkylcelluloses, such as C₁-C₆-alkylcelluloses, including methylcellulose, ethylcellulose and n-propylcellulose; substituted alkylcelluloses, including hydroxy-alkylcelluloses, preferably hydroxy-C₁-C₆-alkylcelluloses and hydroxy-C₁-C₆-alkyl-C₁-C₆-alkylcelluloses, such as hydroxyethylcellulose, hydroxypropylcellulose, hydroxybutylcellulose, hydroxypropylmethylcellulose, ethylhydroxyethylcellulosen and mixtures of the aforementioned, for example the non-ionic polymer is selected from the group consisting of hydroxyalkyl celluloses, preferably from the group consisting of hydroxyethyl cellulose (HEC) and hydroxypropyl cellulose (HPC).

32. The composition according to claim 26, wherein the non-ionic polymer is present in said composition at a concentration of at least 0.8 wt %.

33. The composition according to claim 26, wherein the compound is present in said composition at a concentration of at least 0.08 wt %, or at least 0.1 wt %, or in the range of 0.08 to 3 wt %.

34. The composition according to claim 26, wherein the peptide has a length of 18 to 35 amino acids, or 18-25 amino acids, and comprises or consists of any of the amino acid sequences
(SEQ ID NO 1)
GKYGFYTHVFRLKKWIQKVIDQFGE,
(SEQ ID NO 2)
FYTHVFRLKKWIQKVIDQFGE,
(SEQ ID NO 3)
GKYGFYTHVFRLKKWIQKVI,
(SEQ ID NO 4)
HVFRLKKWIQKVIDQFGE,
(SEQ ID NO: 5)
KYGFYTHVFRLKKWIQKVIDQFGE
(SEQ ID NO: 6)
GKYGFYTHVFRLKKWIQKVIDQF
(SEQ ID NO: 7)
GKYGFYTHVFRLKKWIQKV.

35. The composition according to claim 27, wherein the peptide has a length of 18 to 35 amino acids, or 18-25 amino acids, and comprises or consists of any of the amino acid sequences
(SEQ ID NO 1)
GKYGFYTHVFRLKKWIQKVIDQFGE,
(SEQ ID NO 2)
FYTHVFRLKKWIQKVIDQFGE,
(SEQ ID NO 3)
GKYGFYTHVFRLKKWIQKVI,
(SEQ ID NO 4)
HVFRLKKWIQKVIDQFGE,
(SEQ ID NO: 5)
KYGFYTHVFRLKKWIQKVIDQFGE
(SEQ ID NO: 6)
GKYGFYTHVFRLKKWIQKVIDQF
(SEQ ID NO: 7)
GKYGFYTHVFRLKKWIQKV.

36. The composition according to claim 26, wherein the peptide has at least 90% sequence identity with the amino acid sequence GKYGFYTHVFRLKKWIQKVIDQFGE (SEQ ID NO. 1), or the peptide consists of the amino acid sequence
(SEQ ID NO. 1)
GKYGFYTHVFRLKKWIQKVIDQFGE

37. The composition according to claim 26, wherein EDTA is present in said composition in a concentration of at least 1 mM, or in the range of 1 to 100 mM, or at least 1.5 mM, or at least 2 mM, or in the range of 2 to 100 mM, or in the range of 2 to 50 mM.

38. The composition according to claim 26, wherein the pH of the composition is at the most 8, or lower than 7, or lower than 6, or 5.5 or lower, and/or the pH is higher than 3, or at least 3.5.

39. The composition according to claim 26, wherein the pH of the composition is between 7 and 8, or approx. 7.4.

40. The composition according to claim 26, wherein the composition comprises
at least 90%, such as at least 95% of the initial content of said compound comprising said peptide after storage for 2 months at 37° C.; and/or
at least 75%, such as at least 80%, for example at least 90% of the initial content of said compound comprising said peptide after storage for 4 months at 37° C.; and/or
at least 70%, such as at least 80%, for example at least 85% of the initial content of said compound comprising said peptide after storage for 6 months at 37° C.; and/or
at least 90%, or at least 95% of initial content of said compound comprising said peptide after storage for 8 months at room temperature.

41. A method of treatment of a disorder in an individual in need thereof, wherein the method comprises administration, optionally local administration, of a therapeutically effective amount of the composition according to claim 26.

42. The method according to claim 41, wherein the disorder is a disorder of the skin, ears, eyes or nose.

43. The method according to claim 41, wherein the disorder is a wound, for example a wound selected from the group consisting of burns, non-healing ulcers and surgical wounds.

44. The method according to claim 41, wherein the disorder
comprises an inflammation or is associated with an inflammation and/or
comprises an infection by bacteria or is associated with infection by bacteria, for example an infection by multi-resistant bacteria.

45. The method according to claim 41, wherein the disorder comprises formation of a biofilm.


SEQUENCE LISTING (7 sequences)

SEQ ID NO: 1 - protein, 25 aa, Artificial Sequence
  TCP-25 fragment
  PEPTIDE (1)..(25): TCP-25 fragment
GKYGFYTHVFRLKKWIQKVIDQFGE

SEQ ID NO: 2 - protein, 21 aa, Artificial Sequence
  TCP-25 fragment
  PEPTIDE (1)..(21): TCP-25 fragment
FYTHVFRLKKWIQKVIDQFGE

SEQ ID NO: 3 - protein, 20 aa, Artificial Sequence
  TCP-25 fragment
  PEPTIDE (1)..(20): TCP-25 fragment
GKYGFYTHVFRLKKWIQKVI

SEQ ID NO: 4 - protein, 18 aa, Artificial Sequence
  TCP-25 fragment
  PEPTIDE (1)..(18): TCP-25 fragment
HVFRLKKWIQKVIDQFGE

SEQ ID NO: 5 - protein, 24 aa, Artificial Sequence
  TCP-25 fragment
  PEPTIDE (1)..(24): TCP-25 fragment
KYGFYTHVFRLKKWIQKVIDQFGE

SEQ ID NO: 6 - protein, 23 aa, Artificial Sequence
  TCP-25 fragment
  PEPTIDE (1)..(23): TCP-25 fragment
GKYGFYTHVFRLKKWIQKVIDQF

SEQ ID NO: 7 - protein, 19 aa, Artificial Sequence
  TCP-25 fragment
  PEPTIDE (1)..(19): TCP-25 fragment
GKYGFYTHVFRLKKWIQKV